SEQUENCE LISTING (8 sequences)

SEQ ID NO: 1 - protein, 12 aa, Artificial Sequence
  Description of Artificial Sequence Synthetic peptide
CYGRKKRRQRRR

SEQ ID NO: 2 - protein, 10 aa, Artificial Sequence
  Description of Artificial Sequence Synthetic peptide
CRRRRRRRRR

SEQ ID NO: 3 - protein, 4 aa, Artificial Sequence
  synthetic peptide
RGDC

SEQ ID NO: 4 - protein, 5 aa, Artificial Sequence
  synthetic peptide
RGDFC

SEQ ID NO: 5 - protein, 5 aa, Artificial Sequence
  synthetic peptide
RGDFK

SEQ ID NO: 6 - protein, 19 aa, Artificial Sequence
  synthetic peptide
CYGRKKRRQRRRGGGRGDS

SEQ ID NO: 7 - protein, 4 aa, Artificial Sequence
  synthetic peptide
GLFG

SEQ ID NO: 8 - protein, 4 aa, Artificial Sequence
  synthetic peptide
LALA

(19) United States
(12) Patent Application Publication
(10) Pub. No.: US 2012/0289571 A1
(43) Pub. Date: Nov. 15, 2012

(54) POLYMERIC CONJUGATES OF AROMATIC AMINE CONTAINING COMPOUNDS INCLUDING RELEASABLE UREA LINKER

(75) Inventors: Hong Zhao, Edison, NJ (US); Dechun Wu, Bridgewater, NJ (US); Snehlata Tripathi, Edison, NJ (US); Jing Xia, Warren, NJ (US)

(73) Assignee: ENZON PHARMACEUTICALS, INC., Piscataway, NJ (US)

(21) Appl. No.: 13/520,120

(22) PCT Filed: Dec. 30, 2010
(86) PCT No.: PCT/US10/62609

§ 371 Date: Jun. 29, 2012

Related U.S. Application Data
(60) Provisional application No. 61/291,614, filed on Dec. 31, 2009.
(60) Provisional application No. 61/291,756, filed on Dec. 31, 2009.

(51) Int. Cl. C07D 403/14; A61K 31/404; A61P 35/00; C07D 403/06

(52) U.S. Cl. 514/414; 548/468; 548/460

(57) ABSTRACT

The present invention relates to releasable urea linker systems involving amine-containing chemical compounds and biologically active agents. In particular, the present invention relates to reversibly releasable linkers based on intramolecular cyclization-assisted releasable urea linkages to aromatic amine-containing compounds. The present invention also relates to polymeric conjugates of indolinone-based tyrosine kinase inhibitors.

### CROSS-REFERENCE TO RELATED APPLICATION

This application claims the benefit of priority from U.S. Provisional Patent Application Ser. Nos. 61/291,756 and 61/291,614 filed Dec. 31, 2009, the contents of each of which are incorporated herein by reference.

### FIELD OF THE INVENTION

The present invention relates to releasable urea linker systems involving amine-containing chemical compounds and biologically active agents. In particular, the present invention relates to reversibly releasable linkers based on intramolecular cyclization-assisted releasable urea linkages to aromatic amine-containing compounds. The present invention also relates to polymeric conjugates of indolinone-based tyrosine kinase inhibitors.

### BACKGROUND OF THE INVENTION

Over the years, there have been reports regarding medicinal agents with promising therapeutic activities. However, many of biologically active compounds are insoluble in aqueous fluids and/or rapidly degrade in the body. There have been many attempts to improve pharmaceutical properties of such medicinal agents. One way to overcome the problems of medicinal agents is to attach a modifier to the compounds to provide desired properties, but eventually liberate the biologically active parent compounds. For example, medicinal agents are included as part of prodrugs which, upon administration, regenerate the parent compounds in vivo.

In some situations such as with amine-containing compounds, medicinal agents are attached to a modifier via a hydrolysis-resistant linkage. The resulting compounds are eliminated from the body, before the biologically active parent compounds are regenerated in sufficient amounts in vivo.

Thus, it would be advantageous to provide artisans with alternative and/or improved technologies for reversibly releasable linkages to biologically active agents involving amine-containing compounds.

### SUMMARY OF THE INVENTION

The present invention relates to releasable urea linkers involving amine-containing compounds. In one aspect, there are provided compounds of Formula (I), comprising:

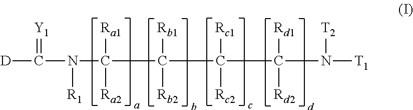

wherein

D is an amine-linked biologically active moiety or a hydroxyl-linked biologically active moiety;

Y₁ is O, S, or NR₅;

R₁ is hydrogen, C₁₋₆ alkyl, or aryl;

Rₐ₁, Rₐ₁, R_{b1}, R_{b2}, R_{c1}, R_{c2}, R_{d1}, and R_{d2}, in each occurrence, are independently selected from among hydrogen, OH, C₁₋₆ alkyls, C₁₋₆ alkenyls, C₁₋₆ alkynyls, C₁₋₆ alkoxy, C₃₋₈ cycloalkyls, aryls, C(O)—R₆, targeting groups, substantially non-antigenic polymers, and

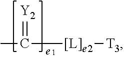

or

two of Rₐ₁, R_{b1}, R_{c1}, and R_{d1} form a four to eight carbon-membered cyclic or heterocyclic ring, and optionally the two of Rₐ₁, R_{b1}, R_{c1}, and R_{d1} form a double bond;

T₁ is selected from among hydrogen, C₁₋₆ alkyls, C₁₋₆ alkenyls, C₁₋₆ alkynyls, C₃₋₈ cycloalkyls, aryls, leaving groups, functional groups, targeting groups, and

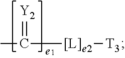

T₂ is selected from among hydrogen, C₁₋₆ alkyls, C₁₋₆ alkenyls, C₁₋₆ alkynyls, C₃₋₈ cycloalkyls, aryls, functional groups, and targeting groups;

Y₂ is O, S, or NR₇;

L, in each occurrence, is the same or different bifunctional linking moiety;

T₃ is selected from among hydrogen, OH, amine, halogen, C₁₋₆ alkyls, C₁₋₆ alkenyls, C₁₋₆ alkynyls, C₁₋₆ alkoxy, C₃₋₈ cycloalkyls, aryls, leaving groups, functional groups, targeting groups, and substantially non-antigenic polymers;

R₅ and R₇ are independently hydrogen, C₁₋₆ alkyl, or aryl;

R₆ is OH, C₁₋₆ alkyl, aryl, C₁₋₆ alkoxy, or aryloxy;

(a), (b), (c), and (d) are independently zero or one, and the sum of (a), (b), (c) and (d) is one, two, three or four; and

(e1) is zero or one;

(e2) is zero or a positive integer of from about 1 to about 6; and

provided that T₁ is

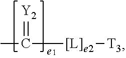

wherein L contains a releasable linker and (e2) is a positive integer of from about 1 to about 6, when T₂ is not hydrogen; and
provided that Rₐ₁, Rₐ₂, R_{b1}, R_{b2}, R_{c1}, R_{c2}, R_{d1}, and R_{d2}, in each occurrence, are not all hydrogen, when T₁ and T₂ are both hydrogen.

In this aspect of the invention, the amine-containing biologically active agent is attached to the C(═Y₁) via the amine.

In one aspect, the present invention provides polymeric conjugates of indolinone-containing tyrosine kinase inhibitors. In one aspect of the invention, compounds of Formula (I′) are provided:

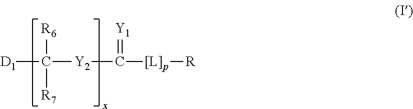

wherein

D₁ is an indolinone-containing kinase inhibitor, wherein D₁ is linked via the indolinone amine;

R is a substantially non-antigenic polymer;

L, in each occurrence, is the same or different bifunctional linker;

R₆ and R₇ are independently hydrogen or C₁₋₄ alkyls;

Y₁ is O, S or NH;

Y₂ is O, S or NH;

(x) is zero or 1; and

(p) is zero or a positive integer of from about 1 to about 6.

Methods of making and using the compounds as well as methods of treatment using the compounds of the present invention are also provided.

In another embodiment, the present invention provides unique reversibly releasable linker systems for compounds containing amines. The amine-containing compounds together with the linker described herein form a urea linkage which undergoes an intramolecular cyclization to regenerate the amine-containing parent compounds.

Advantages will be apparent from the following description and drawings.

One advantage of the present invention is that the intramolecular cyclization-triggered releasable urea linker system is useful in the modification of compounds containing amines, as desired by artisans. In one example, the present invention can be used in the preparation of prodrugs involving aromatic amine-containing compounds. The present invention can be inserted to conjugate amine-containing compounds to polymers which are capable of solubilizing insoluble amine-containing compounds and extending their half-life, as compared to the parent compounds.

Another advantage of the present invention is that additional optional releasable linker(s) can be added to the intramolecular cyclization-assisted releasable urea linker systems. The release of an additional releasable linker can trigger and/or modulate the initiation of the intramolecular cyclization of the present invention. For example, a releasable linker based on a benzyl elimination can facilitate the intramolecular cyclization of the present invention to regenerate parent compounds. The double linker systems can modify the hydrolysis rate for the regeneration of parent compounds.

In a further aspect, the present invention provides a method of delivering an indolinone derivative to a mammal. The method includes (a) forming a polymeric conjugate of an indolinone-based tyrosine kinase inhibitor; and (b) administering the conjugate to a mammal in need thereof, wherein the conjugate is represented by Formula (I).

One advantage of the present invention is that the compounds described herein provide a means for using indolinone-containing tyrosine kinase inhibitors in the treatment of cancer. The compounds employ multi-armed PEGs to load multiple units of the drug molecules through various linkers. The hydrolysis of the parent drugs and the regeneration of the drugs can be modified by linkers as desired by artisans. The polymeric conjugates of the indolinone-containing tyrosine kinase inhibitors can also be formulated with pharmaceutical excipients. In this way, the solubility and bioavailability of indolinone-containing drugs can be improved.

Yet another advantage is that the present invention provides a means for improving pharmacokinetic properties of indolinone-containing tyrosine kinase inhibitors. According to the present invention, water soluble high molecular polymer conjugates of indolinone-based tyrosine kinase inhibitors and related analogs allow improved bioavailability of the indolinone-based tyrosine kinase inhibitor compounds.

Further advantage of the present invention is that patients can be treated concurrently or sequentially with a compound described herein in combination with other anti-cancer therapies for synergistic benefit.

For purposes of the present invention, the term “residue” shall be understood to mean that portion of a compound, to which it refers, i.e. an amine-containing compound, indolinone-containing tyrosine kinase inhibitors, bifunctional linkers, an amino acid, polyethylene glycol, etc. that remains after it has undergone a substitution reaction with another compound.

For purposes of the present invention, the term “polymeric residue,” “polymer containing residue” or “PEG residue” shall each be understood to mean that portion of the polymer or PEG which remains after it has undergone a reaction with, e.g., bifunctional linkers such as amino acids.

For purposes of the present invention, the term “alkyl” refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, and C₁₋₆ alkylcarbonylalkyl groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted the substituted group(s) include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkynyl” refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alklyl group substituted with a C₃₋₈ cycloalkyl group. Examples of cycloalkylalkyl groups include cyclopropylmethyl and cyclopentylethyl.

For purposes of the present invention, the term “alkoxy” refers to an alkyl group of indicated number of carbon atoms attached to the parent molecular moiety through an oxygen bridge. Examples of alkoxy groups include, for example, methoxy, ethoxy, propoxy and isopropoxy.

For purposes of the present invention, an “alkylaryl” group refers to an aryl group substituted with an alkyl group.

For purposes of the present invention, an “aralkyl” group refers to an alkyl group substituted with an aryl group.

For purposes of the present invention, the term “alkoxyalkyl” group refers to an alkyl group substituted with an alkloxy group.

For purposes of the present invention, the term “amino” refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

For purposes of the present invention, the term “halogen’ or “halo” refers to fluorine, chlorine, bromine, and iodine.

For purposes of the present invention, the term “heteroatom” refers to nitrogen, oxygen, and sulfur.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

For purposes of the present invention, the term “heteroaryl” refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as phenyl and napthyl; substituted aryls include moieties such as 3-bromophenyl; aralkyls include moieties such as tolyl; heteroalkyls include moieties such as ethylthiophene; substituted heteroalkyls include moieties such as 3-methoxythiophene; alkoxy includes moieties such as methoxy; and phenoxy includes moieties such as 3-nitrophenoxy.

For purposes of the present invention, “positive integer” shall be understood to include an integer equal to or greater than 1 and as will be understood by those of ordinary skill to be within the realm of reasonableness by the artisan of ordinary skill.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art. An effective amount for each mammal or human patient to be treated is readily determined by the artisan in a range that provides a desired clinical response while avoiding undesirable effects that are inconsistent with good practice. Dose ranges are provided hereinbelow.

For purposes of the present invention, the terms “cancer” and “tumor” are used interchangeably, unless otherwise indicated. “Cancer” encompasses malignant and/or metastatic cancer, unless otherwise indicated.

### BRIEF DESCRIPTION OF THE DRAWINGS

FIG. 1 schematically illustrates a reaction scheme of preparing compounds 5a-c described in Examples 5-7.

FIG. 2 schematically illustrates a reaction scheme of preparing compound 8 described in Examples 8-9.

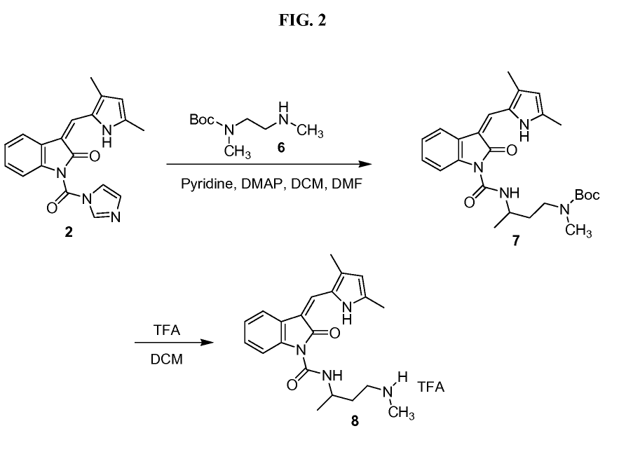

FIG. 3 schematically illustrates a reaction scheme of preparing compounds 10a-c described in Example 10.

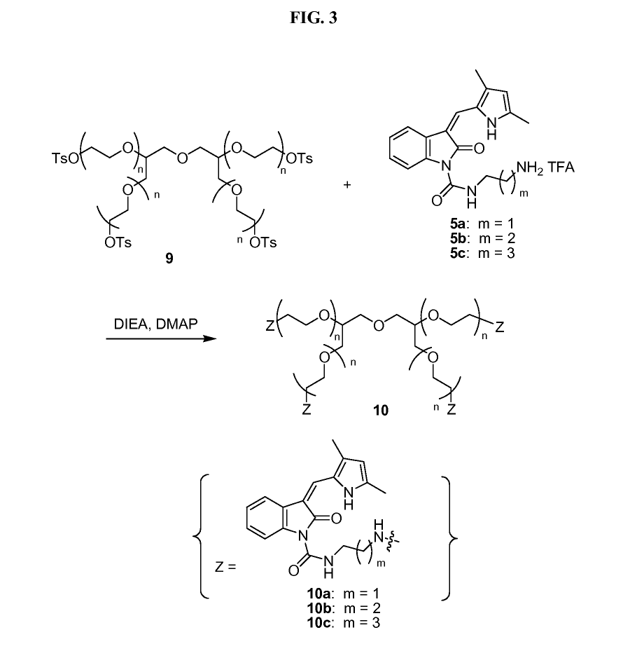

FIG. 4 schematically illustrates a reaction scheme of preparing compound 15 described in Examples 11-13.

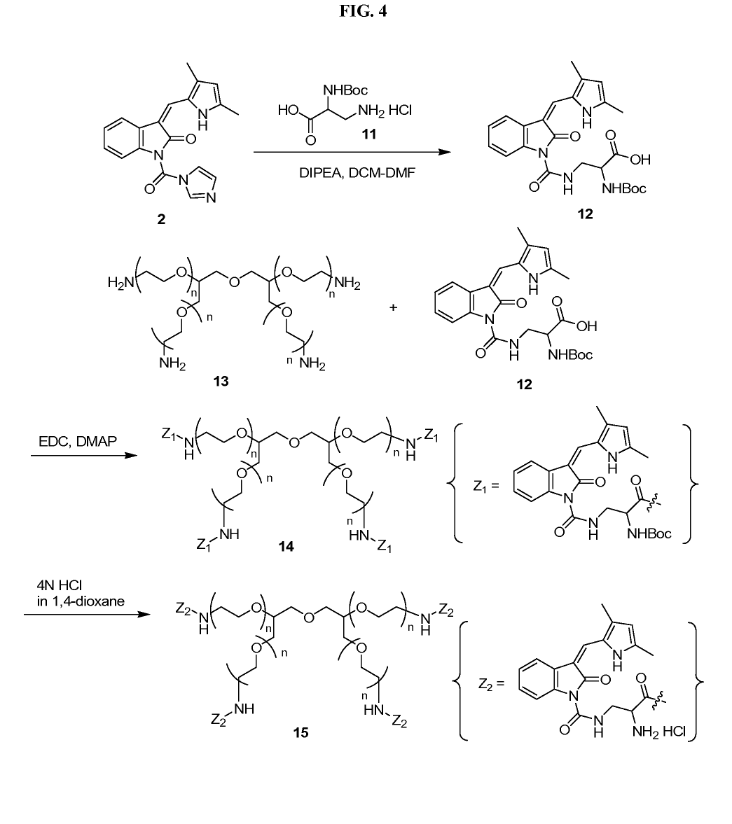

FIG. 5 schematically illustrates a reaction scheme of preparing compounds 17a-d described in Example 14.

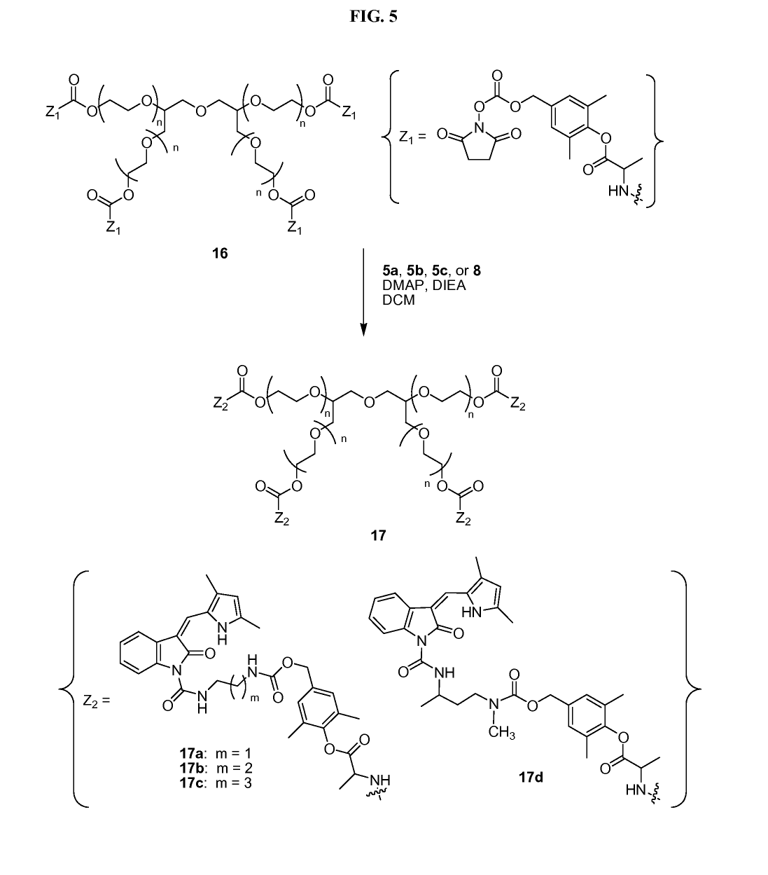

FIG. 6 schematically illustrates a reaction scheme of preparing compounds 19a-d described in Example 15.

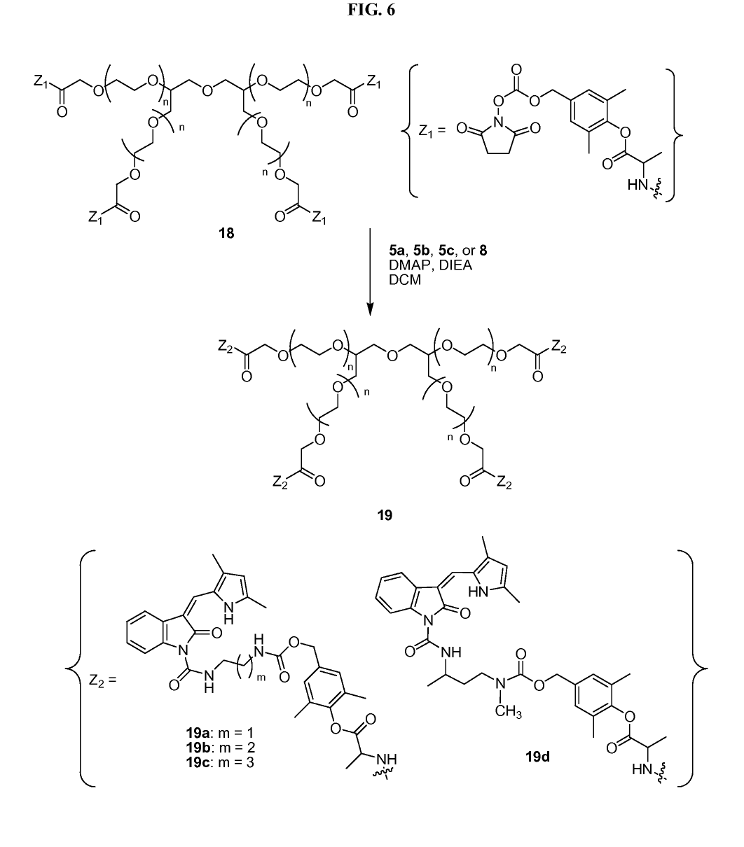

FIG. 7 schematically illustrates a reaction scheme of preparing compound 23 described in Examples 16-18.

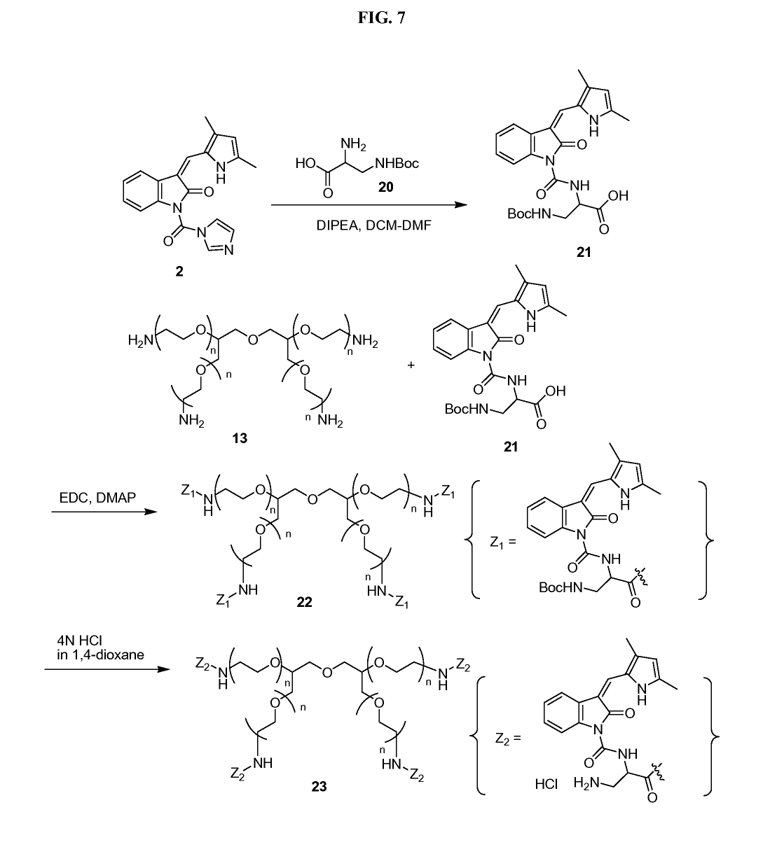

FIG. 8 schematically illustrates a reaction scheme of preparing compounds 25a-d described in Example 19.

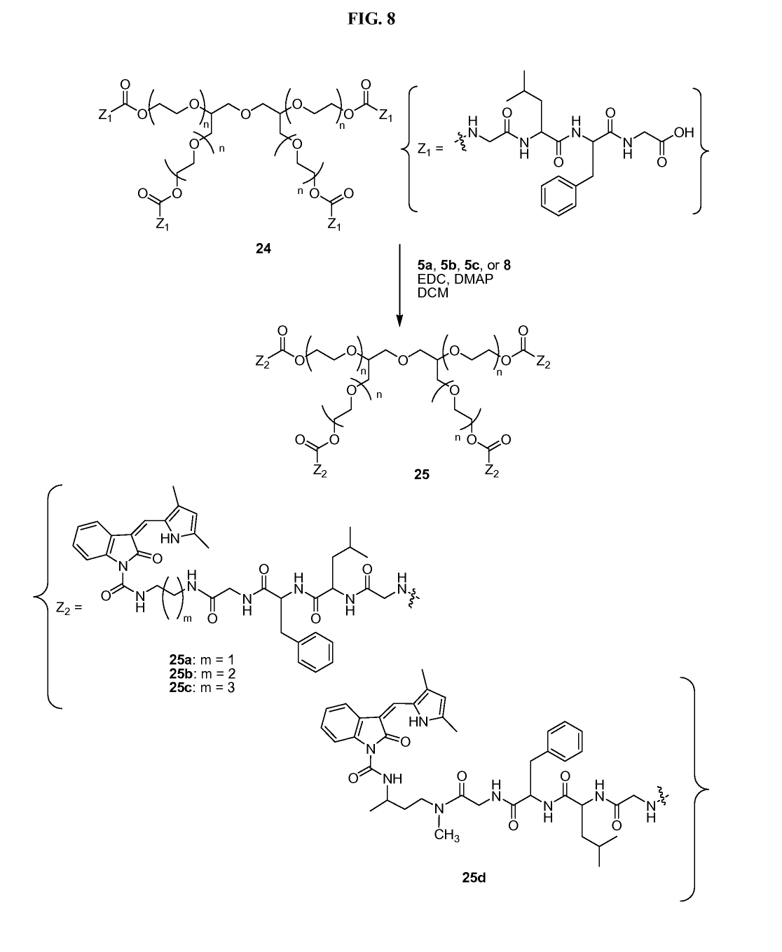

FIG. 9 schematically illustrates a reaction scheme of preparing compounds 27a-d described in Example 20.

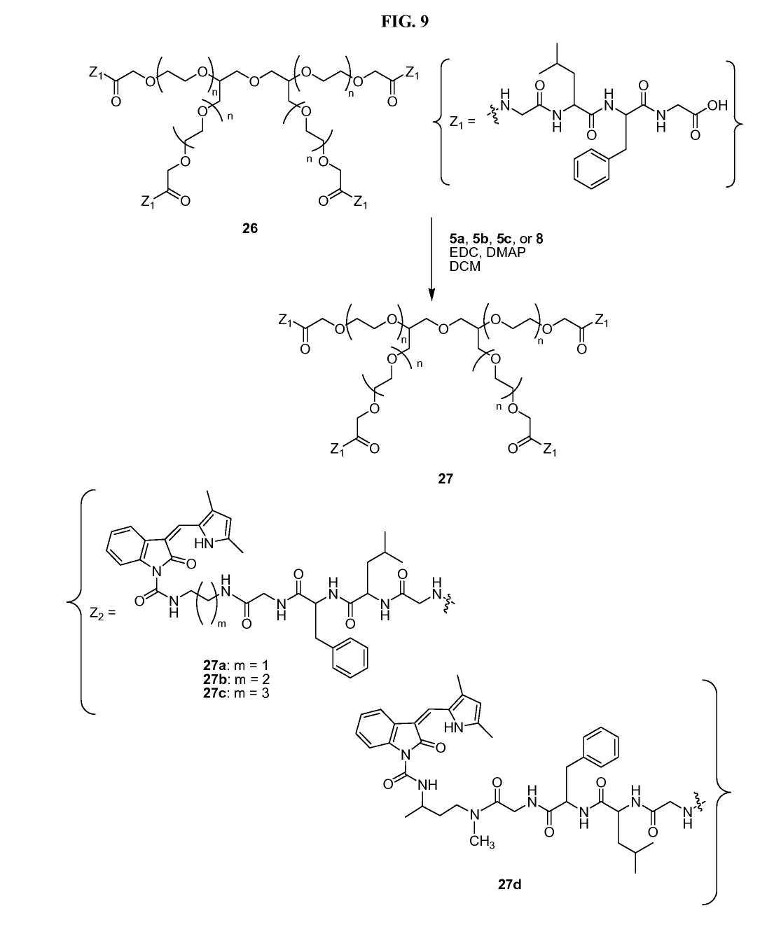

FIG. 10 schematically illustrates a reaction scheme of preparing compounds 34a-b described in Examples 21-23.

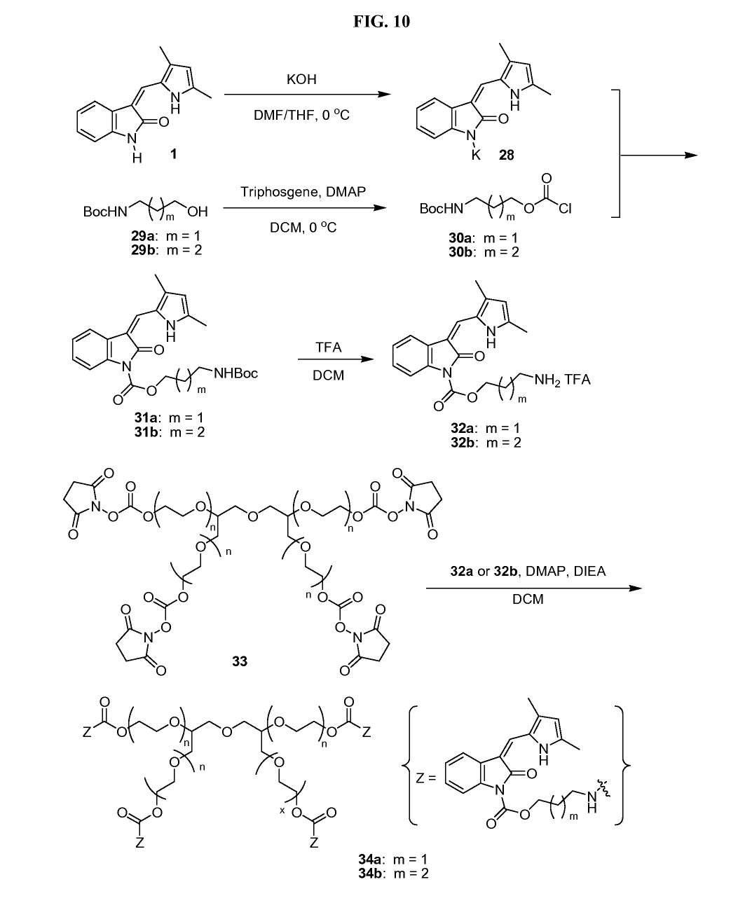

FIG. 11 schematically illustrates a reaction scheme of preparing compound 39 described in Examples 24-26.

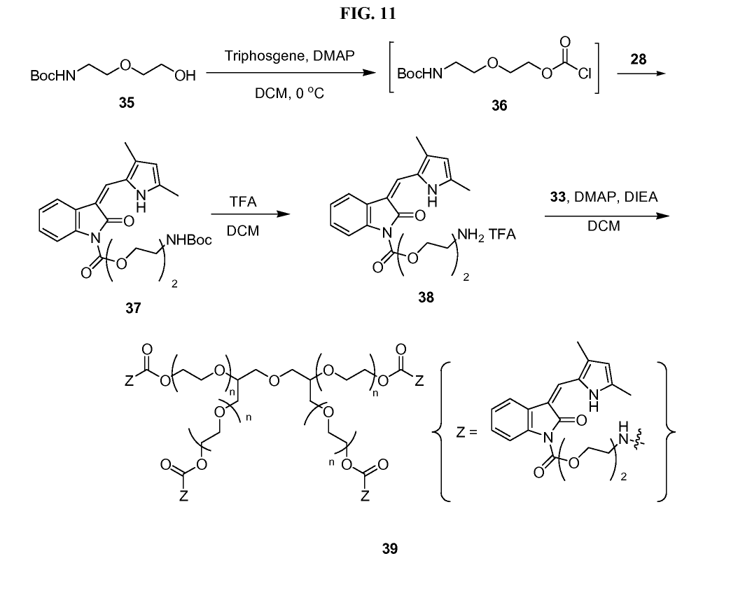

FIG. 12 schematically illustrates a reaction scheme of preparing compounds 44a-b described in Examples 27-29.

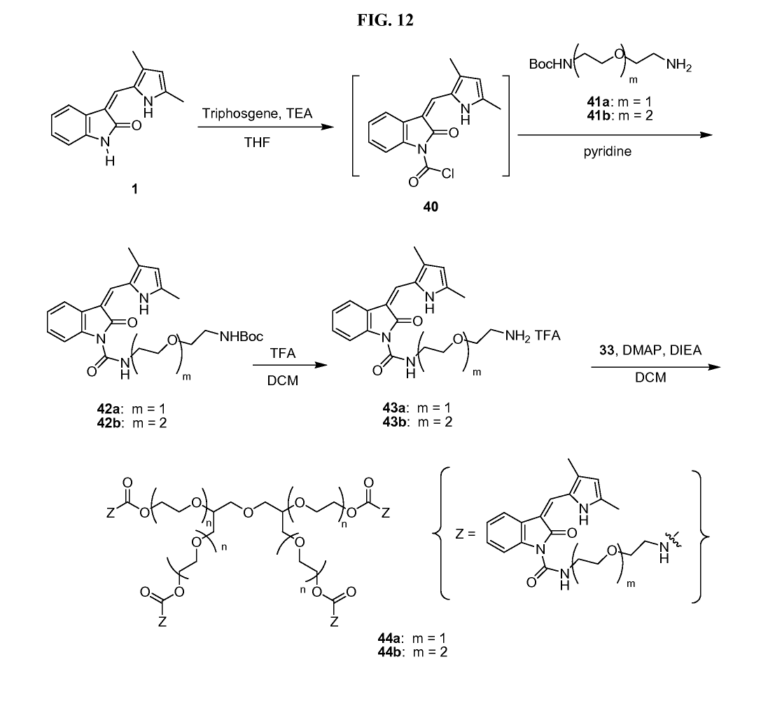

FIG. 13 schematically illustrates a reaction scheme of preparing compounds 48a-d described in Examples 30-32.

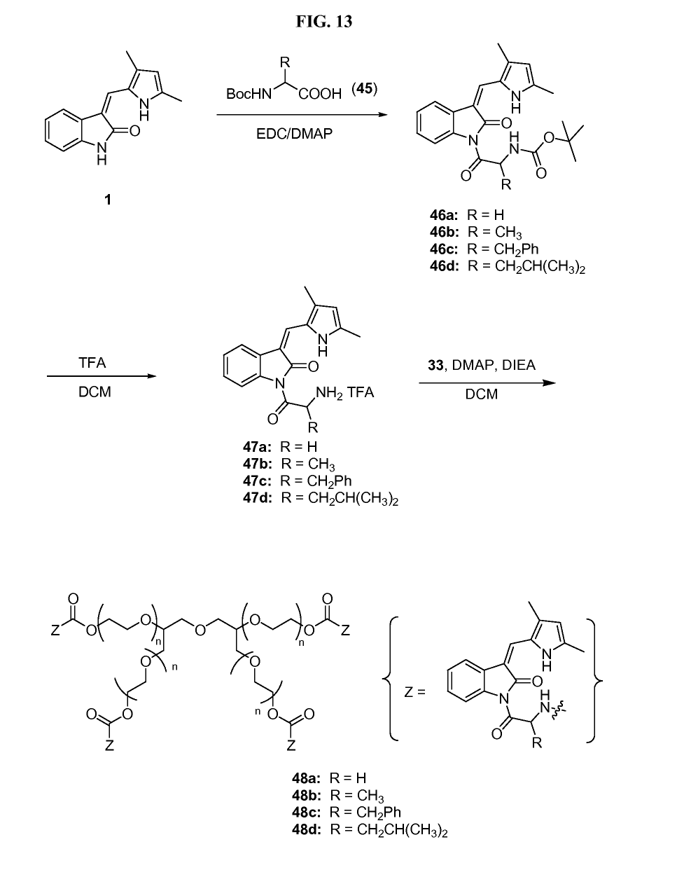

FIG. 14 schematically illustrates a reaction scheme of preparing compounds 50a-h described in Example 33.

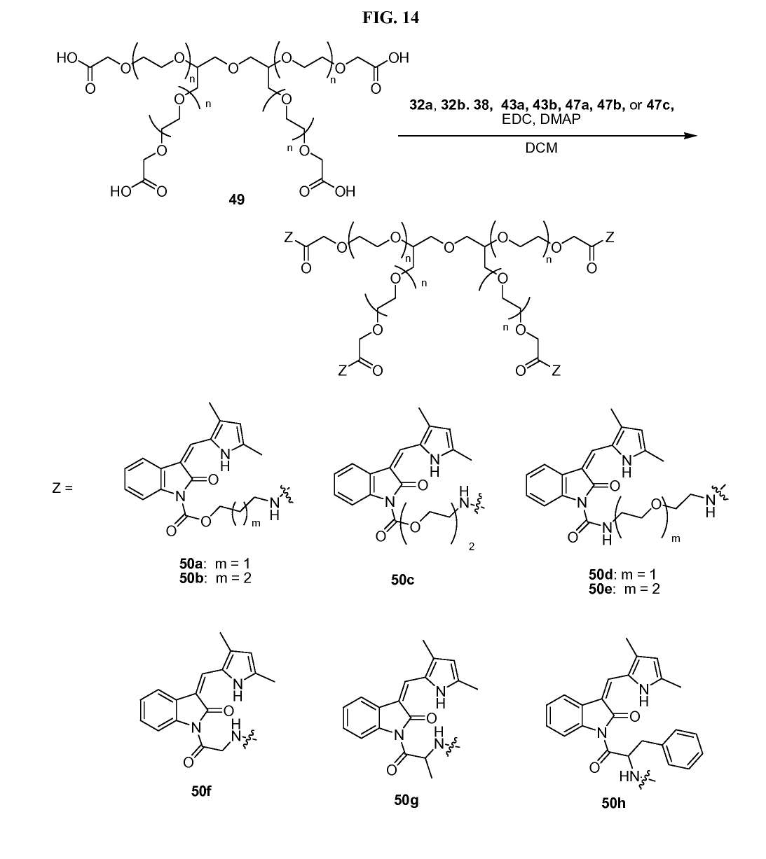

FIG. 15 schematically illustrates a reaction scheme of preparing compound 53 described in Examples 34-35.

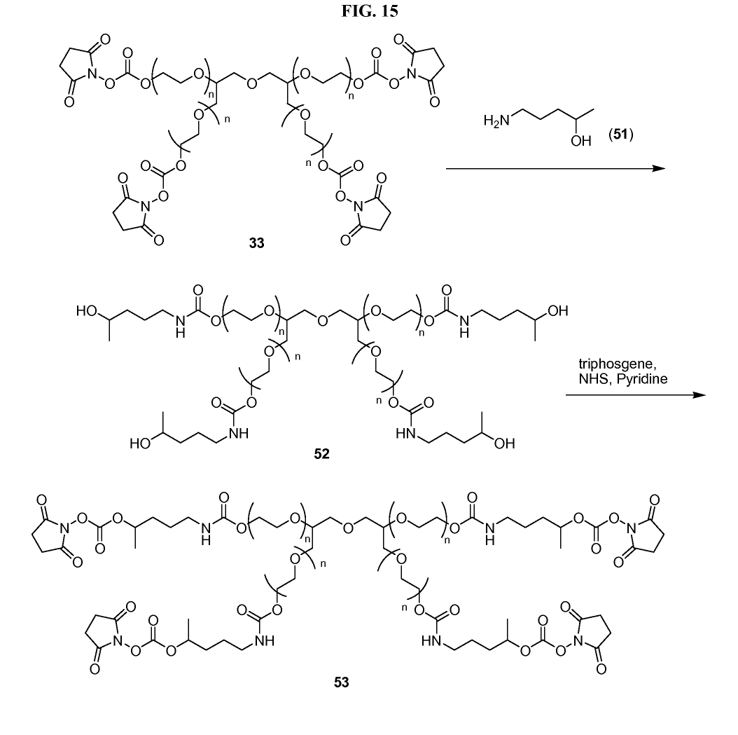

FIG. 16 schematically illustrates a reaction scheme of preparing compound 54 described in Example 36.

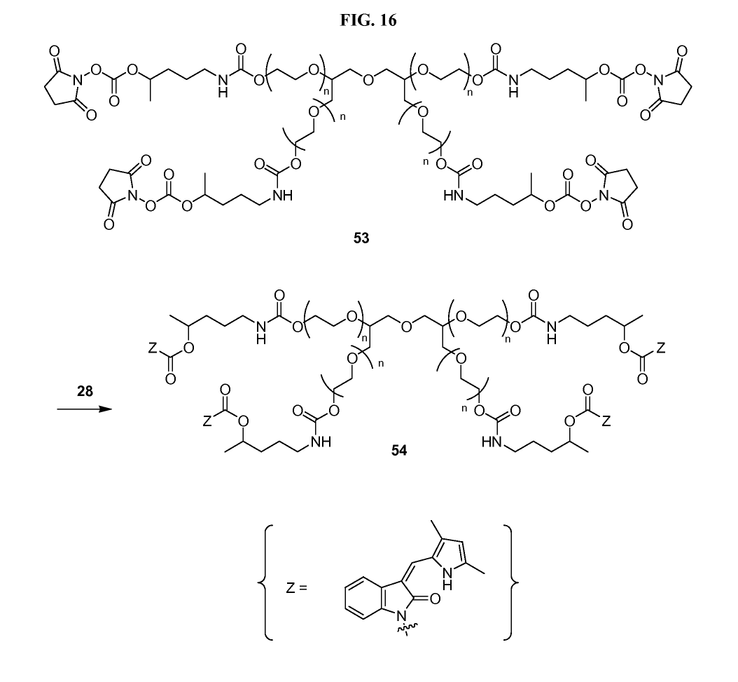

FIG. 17 schematically illustrates a reaction scheme of preparing compound 56 described in Examples 37-38.

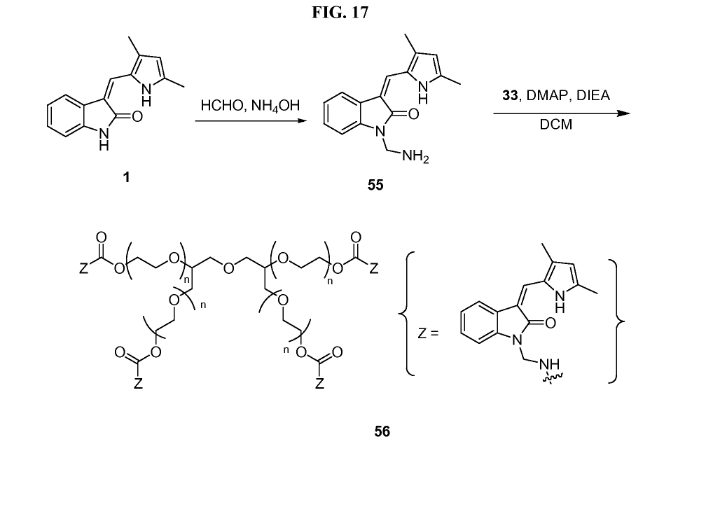

FIG. 18 schematically illustrates a reaction scheme of preparing compound 60 described in Examples 39-42.

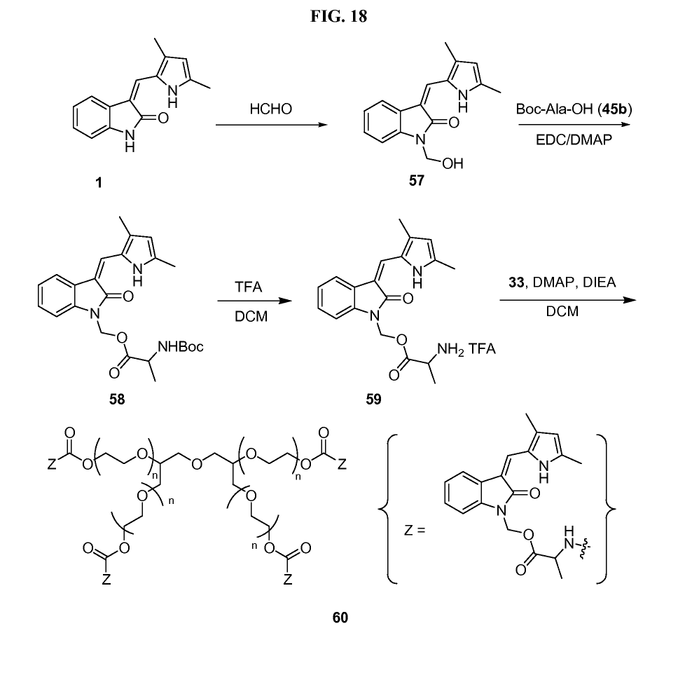

FIG. 19 schematically illustrates a reaction scheme of preparing compound 61 described in Example 43.

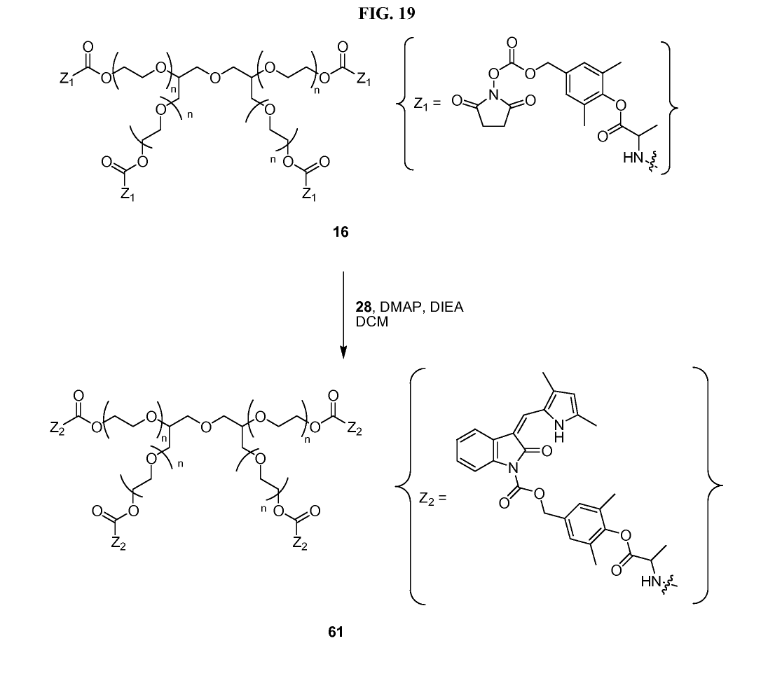

FIG. 20 schematically illustrates a reaction scheme of preparing compound 62 described in Example 44.

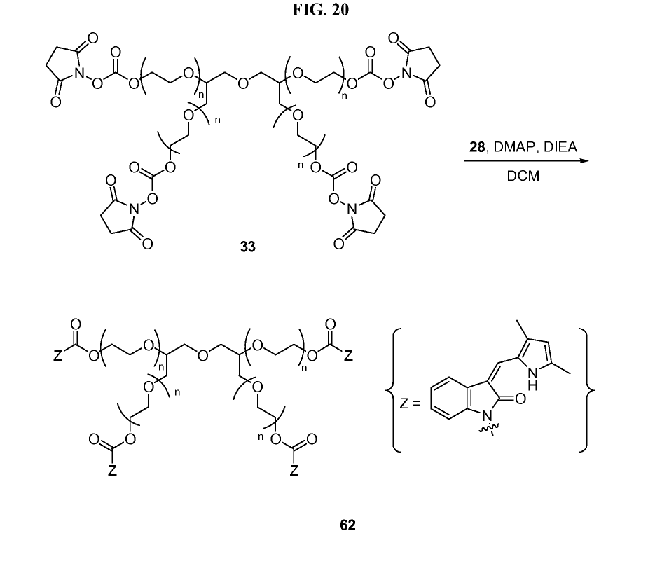

### DETAILED DESCRIPTION OF THE INVENTION

### A. Overview

In one embodiment of the present invention, there are provided compounds of Formula (I):

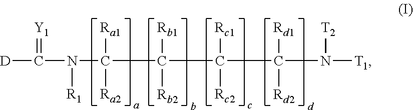

wherein

D is an amine-linked biologically active moiety or a hydroxyl-linked biologically active moiety;

Y₁ is O, S, or NR_{S};

R₁ is hydrogen, C₁₋₆ alkyl, or aryl;

Rₐ₁, Rₐ₂, R_{b1}, R_{b2}, R_{c1}, R_{c2}, R_{d1}, and R_{d2}, in each occurrence, are independently selected from among hydrogen, OH, C₁₋₆ alkyls, C₁₋₆ alkenyls, C₁₋₆ alkynyls, C₁₋₆ alkoxy, C₃₋₈ cycloalkyls, aryls, C(O)—R₆, targeting groups, substantially non-antigenic polymers, and

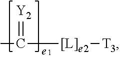

or two of Rₐ₁, R_{b1}, R_{c1}, and R_{d1} form a four to eight carbon-membered cyclic or heterocyclic ring, and optionally the two of Rₐ₁, R_{b1}, R_{c1}, and R_{d1} form a double bond;

T₁ is selected among hydrogen, C₁₋₆ alkyls, C₁₋₆ alkenyls, C₁₋₆ alkynyls, C₃₋₈ cycloalkyls, aryls, leaving groups, functional groups, targeting groups, and

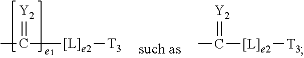

T₂ is selected from among hydrogen, C₁₋₆ alkyls, C₁₋₆ alkenyls, C₁₋₆ alkynyls, C₃₋₈ cycloalkyls, aryls, functional groups, and targeting groups;

Y₂ is O, S, or NR₇;

L, in each occurrence, is the same or different bifunctional linking moiety, which can be a permanent or releasable linker;

T₃ is selected from among hydrogen, OH, amine, halogen, C₁₋₆ alkyls, C₁₋₆ alkenyls, C₁₋₆ alkynyls, C₁₋₆ alkoxy, C₃₋₈ cycloalkyls, aryls, leaving groups, functional groups, targeting groups, and substantially non-antigenic polymers;

R₅ and R₇ are independently hydrogen, C₁₋₆ alkyl, or aryl;

R₆ is OH, C₁₋₆ alkyl, aryl, C₁₋₆ alkoxy, or aryloxy;

(a), (b), (c), and (d) are independently zero or one, and the sum of (a), (b), (c) and (d) is one, two, three or four, preferably two; and

(e1) is zero or one, preferably 1;

(e2) is zero or a positive integer of from about 1 to about 6 (e.g., 1, 2, 3, 4, 5, 6); and

provided that T₁ is

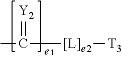

or a leaving group, wherein L contains a releasable linker and (e2) is a positive integer of from about 1 to about 6, when T₂ is not hydrogen; and provided that Rₐ₁, Rₐ₂, R_{b1}, R_{b2}, R_{c1}, R_{c2}, Rₐ₁, and R_{d2}, in each occurrence, are not all hydrogen, when T₁ and T₂ are both hydrogen.

In this aspect, the NT₁T₂ moiety is not attached to the carbon which is present at the distal end from C(═Y₁), when T₁ and T₂ are each hydrogen, and the remaining Rₐ₁, Rₐ₂, R_{b1}, R_{b2}, R_{c1}, R_{c2}, R_{d1}, and R_{d2}, in each occurrence, are all hydrogen.

According to the present invention, the compounds containing a reversible linkage based on an intramolecular cyclization-assisted releasable urea linkage can have the following formula:

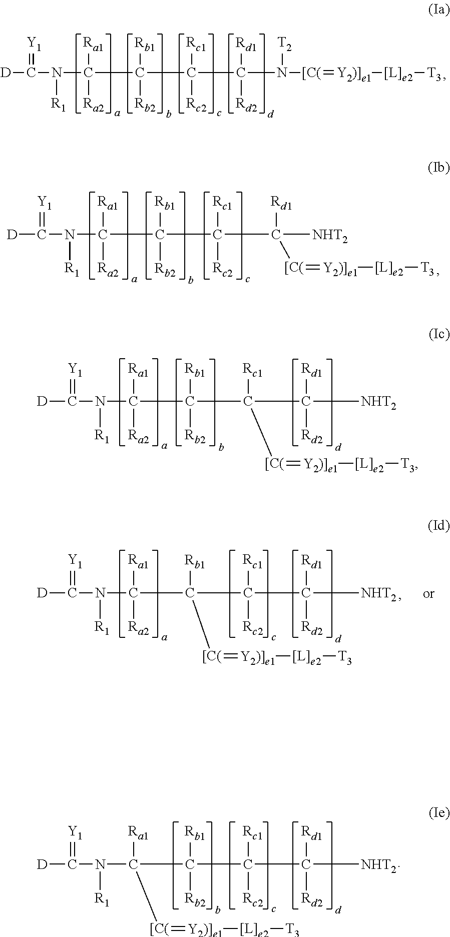

In the compounds represented by formula (Ia), L includes a releasable linker and (e2) is a positive integer, when T₂ is not hydrogen. The L group, in each occurrence, can be a permanent or releasable bifunctional linker, when T₂ is hydrogen.

In certain aspects, the present invention is provided in which the compounds have the formula:

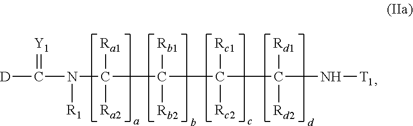

wherein T₁ is selected from among hydrogen, C₁₋₆ alkyls, C₁₋₆ alkenyls, C₁₋₆ alkynyls, C₃₋₈ cycloalkyls, aryls, leaving groups, functional groups, targeting groups,

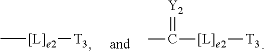

In this aspect, T₂ is hydrogen and the L linker can be a permanent or releasable bifunctional linker. Preferably, T₁ is a leaving group, a functional group, a targeting group,

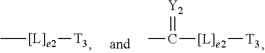

wherein T₃ is selected from among hydrogen, OH, halogen, C₁₋₆ alkyls, C₁₋₆ alkenyls, C₁₋₆ alkynyls, C₁₋₆ alkoxy, C₃₋₈ cycloalkyls, aryls, leaving groups, functional groups, targeting groups and substantially non-antigenic polymers.

In one particular embodiment, one or more of Rₐ₁, Rₐ₂, R_{b1}, R_{b2}, R_{c1}, R_{c2}, Rₐ₁, and R_{d2}, in each occurrence, are the same or different and selected from among hydrogen, OH, C₁₋₆ alkyls, C₁₋₆ alkenyls, C₁₋₆ alkynyls, C₁₋₆ alkoxy, C₃₋₈ cycloalkyls, aryls, C(O)—R₆, and targeting groups. In another particular embodiment, T₃ includes a substantially non-antigenic polymer.

In certain aspects, the present invention is provided in which the compounds have the formula:

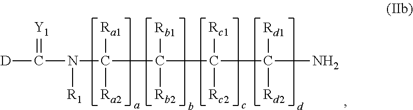

wherein

one or more (e.g., 1, 2) of Rₐ₁, Rₐ₂, R_{b1}, R_{b2}, R_{c1}, R_{c2}, Rₐ₁, and R_{d2}, is selected from among targeting groups, substantially non-antigenic polymers, and

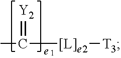

and

T₃ is selected from among hydrogen, OH, amine, halogen, C₁₋₆ alkyls, C₁₋₆ alkenyls, C₁₋₆ alkynyls, C₁₋₆ alkoxy, C₃₋₈ cycloalkyls, aryls, leaving groups, functional groups, targeting groups and substantially non-antigenic polymers, wherein T₃ is not hydrogen (preferably, not hydrogen or C₁₋₆ alkyl), when (e1) and (e2) are each zero.

In this aspect, T₁ and T₂ are both hydrogen. Preferably, one or more (e.g., 1, 2) of Rₐ₁, Rₐ₂, R_{b1}, R_{b2}, R_{c1}, R_{c2}, R_{d1}, and R_{d2} is

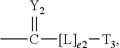

wherein T₃ is selected from among halogen, C₁₋₆ alkyls, C₁₋₆ alkenyls, C₁₋₆ alkynyls, C₁₋₆ alkoxy, C₃₋₈ cycloalkyls, aryls, leaving groups, functional groups, targeting groups and substantially non-antigenic polymers, preferably substantially non-antigenic polymers.

In one aspect of the present invention, there are provided compounds of Formula (I′):

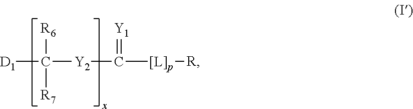

wherein

D₁ is an indolinone-containing kinase inhibitor, wherein D₁ is linked via the indolinone amine;

R is a substantially non-antigenic polymer;

L, in each occurrence, is the same or different bifunctional linker;

R₆ and R₇ are independently hydrogen or C₁₋₄ alkyls;

Y₁ is O, S or NH, preferably 0;

Y₂ is O, S or NH, preferably 0;

(x) is zero or 1, preferably zero; and

(p) is zero or a positive integer of from about 1 to about 6, preferably 1, 2, 3.

In certain aspects, the compound has the formula:

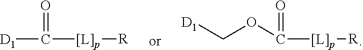

In one preferred aspect, there are provided compounds of Formula (II′):

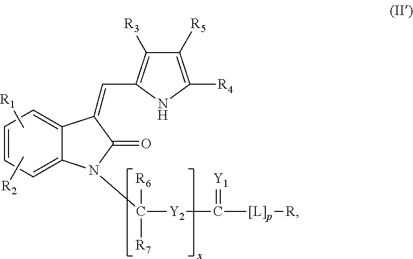

wherein

R is a substantially non-antigenic polymer;

L, in each occurrence, is the same or different bifunctional linker;

R₁ and R₂ are independently selected from among hydrogen, halogen, alkyls, alkylthio, nitro, trihalomethyl, hydroxy, hydroxyalkyls, alkoxys, cyano, aryl, —C(O)R₁₁, NR₁₂R₁₃, —NR₁₂C(O)R₁₃, —SO₂R₁₂, and —S(O)₂NR₁₂R₁₃,
wherein R₁₁ is selected from among alkyls, amino, hydroxy, alkoxys, aryl, aryloxy, and aminoalkylamino; and R₁₂ and R₁₃ are independently selected from among hydrogen, alkyls, and aryl;

R₃ is selected from among hydrogen, alkyls (preferably, methyl), hydroxyalkyls, aminoalkyls, —C(O)R₁₁, and aryl;

R₄ is selected from among hydrogen, alkyls (preferably methyl), —C(O)R₁₁, and aryl;

R₅ is selected from among hydrogen, —CH₂CH₂COOH, —COR₁₄, and —CH₂CH₂C(O)NR₁₅R₁₆, wherein
(a) when R₅ is —COR₁₄, R₁₄ is selected from among alkyls, alkoxys, hydroxy, aryl, aryloxy, alkylamino, dialkylamino, and —NR₃₁R₃₂,
   wherein R₃₁ is hydrogen or an alkyl; and R₃₂ is selected from among aminoalkyls, hydroxyalkyls, acetylalkyls, cyanoalkyls, carboxyalkyls, and alkoxycarbonylalkyls; and wherein the alkyl in the aminoalkyls is optionally substituted with one or two hydroxyl group(s); and
(b) when R₅ is —CH₂CH₂C(O)NR₁₅R₁₆,
   (i) R₁₅ is hydrogen or a C₁₋₄ alkyl; and R₁₆ is -A₁-NR₃₃R₃₄,
      wherein R₃₃ and R₃₄ are independently hydrogen or C₁₋₄ alkyls; and A₁ is (CH₂)ₐ₁, (CH₂)ₐ₂-A₂-(CH₂)ₐ₃ or (CH₂CH₂O)ₐ₄CH₂CH₂, wherein (a1) is an integer of from about 2 to about 10 (e.g., 2, 3, 4, 5, 6, 7, 8); (a2) and (a3) are independently selected integers of from about 1 to about 6 (e.g., 1, 2, 3, 4, 5, 6); A₂ is CH═CH, phenylene, biphenylene, cyclohexylene or piperazinylene; and (a4) is 1, 2 or 3; or
   (ii) R₁₅ and R₁₆ together form -A₃-NR₃₅-A₄-,
      wherein R₃₅ is hydrogen or a C₁₋₄ alkyl; and A₃ and A₄ are independently (CH₂)ₐ₅ or (CH₂CH₂O)ₐ₆CH₂CH₂, wherein (a5) is an integer of from about 2 to about 6 (e.g., 2, 3, 4, 5, 6); and (a6) is 1, 2 or 3; or
   (iii) R₁₅ and R₁₆ together with the nitrogen atom to which they are attached form a piperidinyl, wherein the piperidinyl group bears a substituent of formula -A₅-R₃₆ at the 4 position, wherein A₅ is C₁₋₄ alkylene; and R₃₆ is piperidin-4-yl; or
   (iv) R₁₅ and R₁₆ together with the nitrogen atom to which they are attached form pyrrolidinyl, piperidinyl or morpholino; or

R₄ and R₅ together form —(CH₂)₄— or —(CH₂)ₐ₇CO(CH₂)ₐ₈—, wherein (a7) is 0, 1, 2, or 3; (a8) is 0, 1, 2, or 3, provided that the sum of (a7) and (a8) is 3;

R₆ and R₇ are independently hydrogen or C₁₋₄ alkyls;

Y₁ is O, S or NH, preferably 0;

Y₂ is O, S or NH, preferably 0;

(x) is zero or 1; and

(p) is zero or a positive integer of from about 1 to about 6, preferably 1, 2, 3.

In one embodiment, the compound of Formula (I) are provided in which R₁ and R₂ are independently hydrogen, methyl, or ethyl; R₃ and R₄ are both methyl; and R₅ is hydrogen, or —CH₂CH₂COOH.

The compounds described herein can include polymers. The compounds including polymers can be selected from:

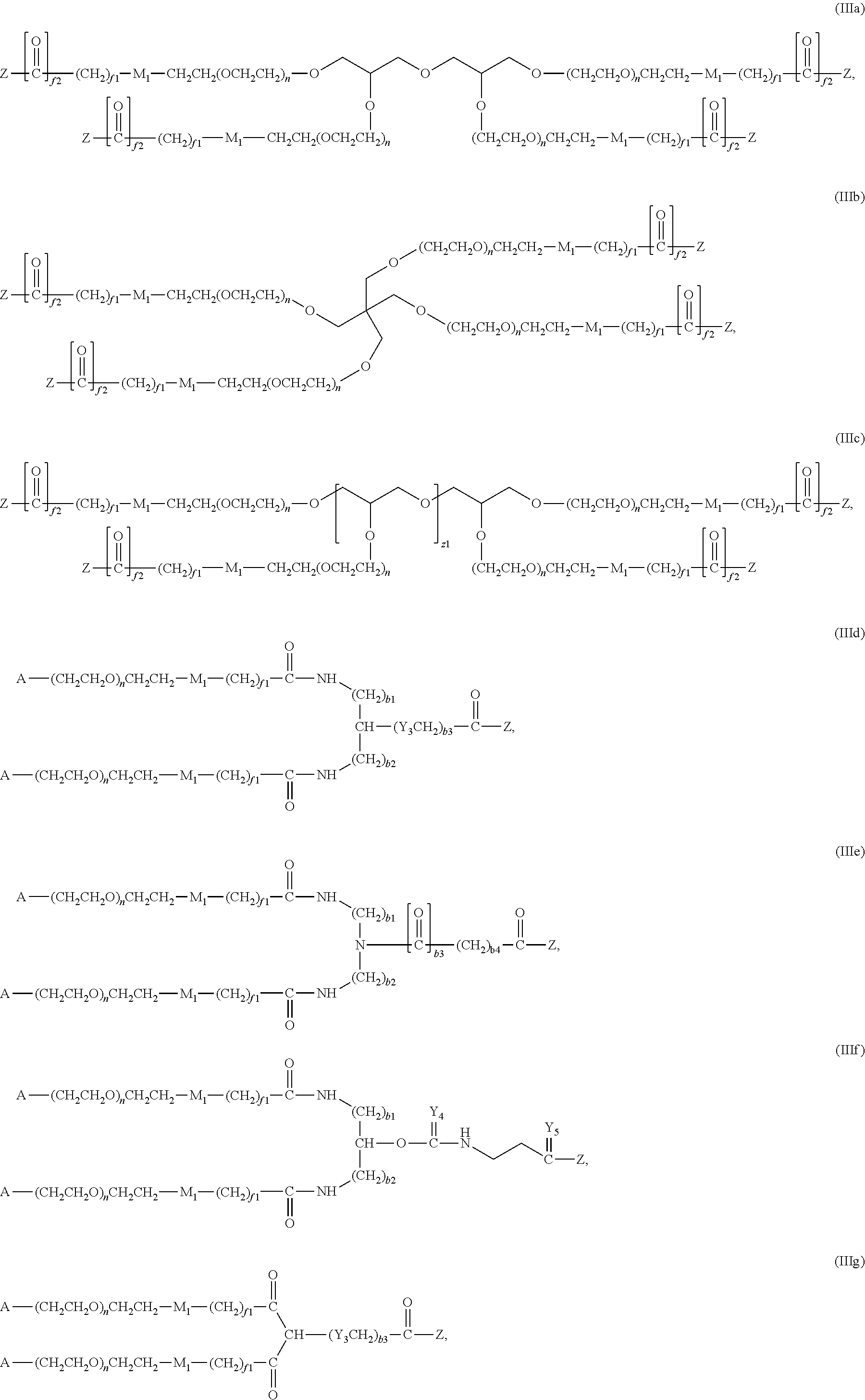

Z—[C(═O)]_{f2}—(CH₂)_{f1}-M₁-CH₂CH₂—O—(CH₂CH₂O)ₙ—CH₂CH₂-M₁-(CH₂)_{f1}-[C(═O)]_{f2}—Z, (IIIh)

and

A-(CH₂CH₂O)ₙ—CH₂CH₂-M₁-(CH₂)_{f1}-[C(═O)]_{f2}—Z, (IIIi)

wherein

A is hydroxyl, NH₂, CO₂H, or C₁₋₆ alkoxy;

M₁ is O, S, or NH;

Y₃ is O, NR₅₁, S, SO or SO₂;

Y₄ and Y₅ are independently O, S or NR₅₁;

R₅₁, in each occurrence, is independently hydrogen, C₁₋₈ alkyl, C₁₋₈ branched alkyl, C₁₋₈ substituted alkyl, aryl, or aralkyl;

Z, in each occurrence, is independently OH, a leaving group, a targeting group, C₁₋₈ alkyl, C₁₋₈ alkoxy, an aryl,

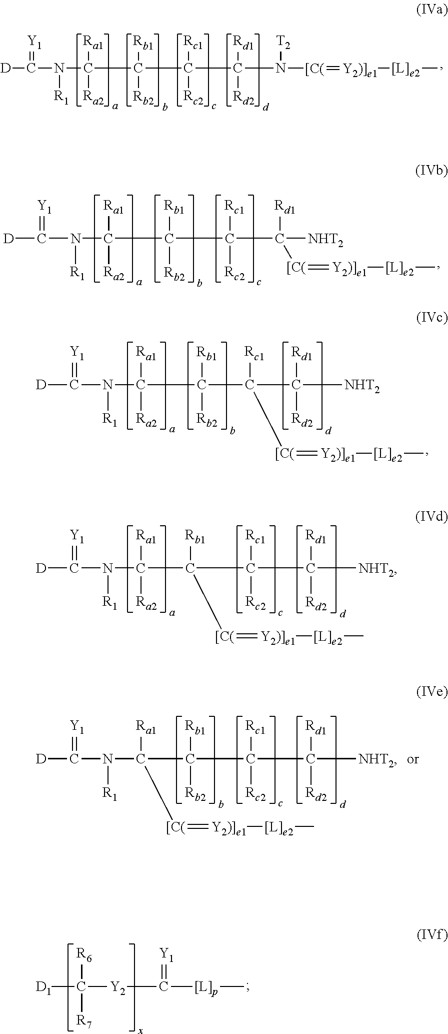

wherein, T₂ is selected from among hydrogen, C₁₋₆ alkyls, C₁₋₆ alkenyls, C₁₋₆ alkynyls, C₃₋₈ cycloalkyls, and aryls;

(b1) and (b2) are independently zero or positive integers, preferably zero or an integer from about 1 to about 5 (e.g., 1, 2, 3, 4, 5);

(b3) is zero or one;

(b4) is a positive integer, preferably, an integer of from 1 about 10 (e.g., 1, 2, 3, 4, 5, 6);

(f1) is zero or a positive integer of from about 1 to about 10, preferably, 0, 1, 2, or 3, and more preferably, zero or one;

(f2) is zero or one, preferably one;

(z1) is zero or a positive integer of from about 1 to about 27, preferably an integer from about 1 to about 13, (e.g., 1, 5, 13);

(n) is a positive integer of from about 10 to about 2,300 so that the polymeric portion of the compound has the total number average molecular weight of from about 2,000 to about 100,000 daltons;

(x) is zero or 1;

(p) is zero or a positive integer of from about 1 to about 6, preferably 1, 2, 3;

and

all other variables are the same as defined in the above;

provided that one or more Z are (IVa), (IVb), (IVc), (IVd), (IVe) or (IVf).

According to the present invention, compounds of Formula (I) described herein include:

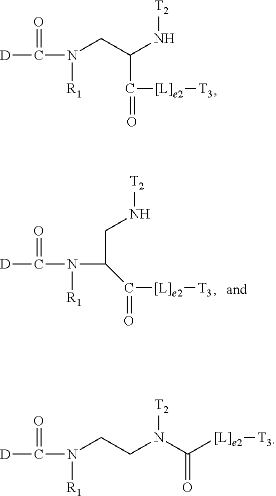

In one particular embodiment, R₁ is hydrogen and the compounds are selected from among:

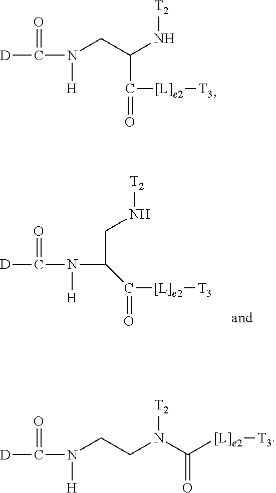

In one preferred embodiment, Z, in each occurrence, is:

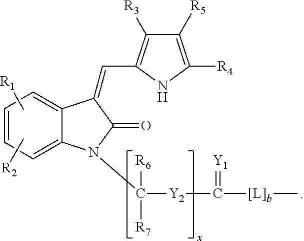

In one particular embodiment, there are provided compounds of Formula (I), wherein Z is

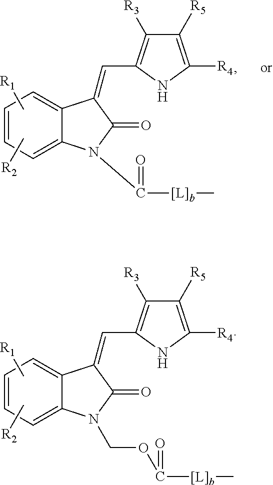

For example, Z is selected from among:

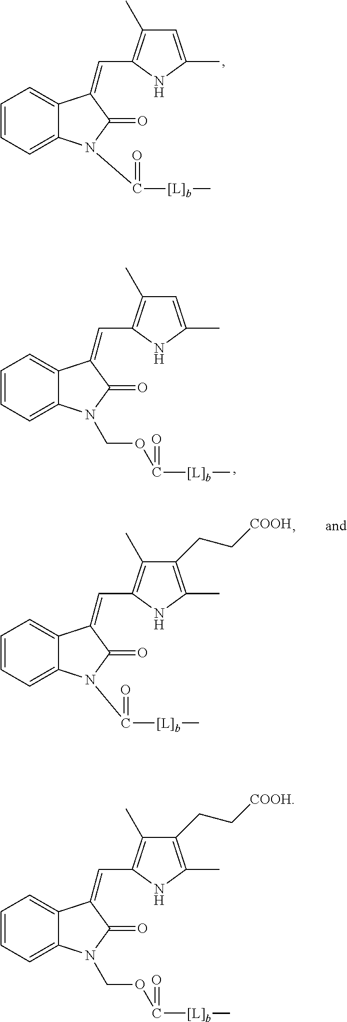

In one preferred embodiment, the compounds described herein have the structure

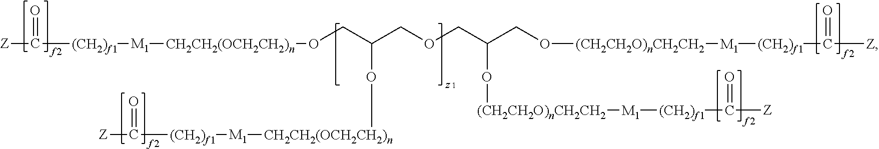

wherein

M₁ is O, S, or NH;

(f1) is zero or a positive integer of from about 1 to about 10;

(f2) is zero or 1;

(z1) is zero or a positive integer of from 1 to about 27;

(n) is a positive integer of from about 10 to about 2,300; and

Z, in each occurrence, is independently OH, a leaving group, a targeting group, C₁₋₈ alkyl, C₁₋₈ alkoxy, an aryl, or

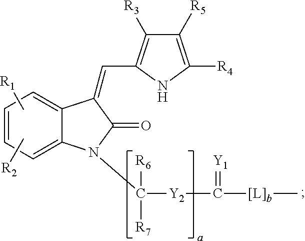

provided that one or more (up to four) Z are

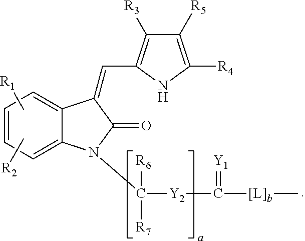

Preferably, the degree of polymerization for the polymer (n) is from about 28 to about 341 to provide polymers having a total number average molecular weight of from about 5,000 Da to about 60,000 Da, and preferably from about 114 to about 239 to provide polymers having a total number average molecular weight of from about 20,000 Da to about 42,000 Da. In one particularly preferred embodiment of the invention, (n) is about 227 to provide the polymeric portion having a total number average molecular weight of about 40,000 Da.

### B. Aromatic Amine-Containing Biologically Active Agents

According to the present invention, the biologically active agents can be hydroxyl- or amine-containing compounds, including pharmaceutically active agents (small molecules having an average molecular weight of less than about 1,500 daltons (e.g., less than about 1,000 daltons), peptides, proteins, nucleic acids, etc. The present invention is useful for modifying vinyl amine-containing compounds. Preferably, the present invention is useful for providing a reversibly releasable linker to aromatic amine-containing compounds. The aromatic amine-containing compounds refer to molecules which include an amine attached to a vinyl group, which is preferably part of an aryl ring including a heteroaryl ring, represented by the structure:

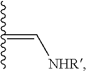

wherein R′ can be hydrogen, alkyl, aryl, or acyl.

One preferred aspect is that biologically active compounds contemplated are aromatic amine-containing biologically active agents, preferably heteroaromatic amine-containing compounds. For example, the biologically active agents include, but are not limited to, indolinone-containing biologically active agents (e.g., SU5416 and derivatives), indole-containing biologically active agents, purine-containing biologically active agents (e.g., toyocamycin), and pyrimidine-containing biologically active agents. Other art-known compounds containing aromatic amines are contemplated within the compounds described herein.

Among aromatic amine-containing biologically active agents, a non-limited example is represented by the following:

(i) indolinone-containing biologically active agents refer to a molecule having the structure:

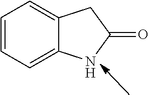

(ii) indole-containing biologically active agents refer to a molecule having the structure:

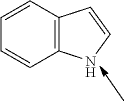

(iii) purine-containing biologically active agents refer to a molecule having the structure:

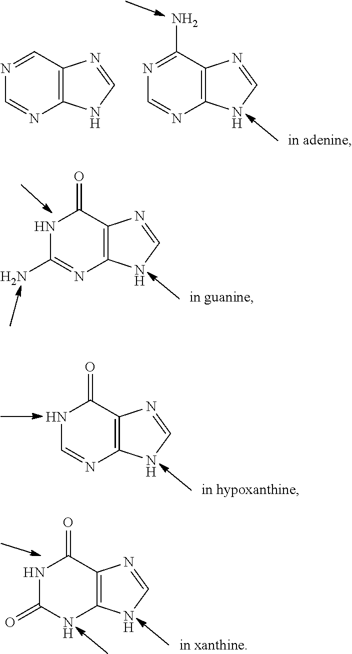

(iv) pyrimidine-containing biologically active agents refer to a molecule having the structure:

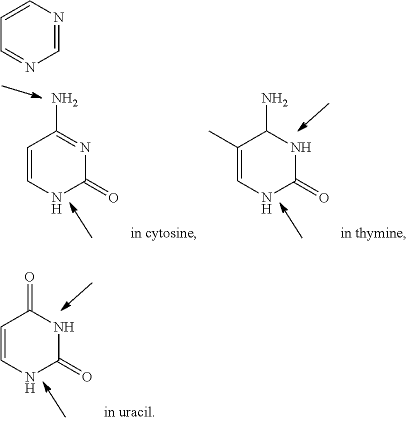

In the above examples, the arrow(s) indicate aromatic amine(s) which can be linked to a releasable urea linker according to the present invention.

According to the present invention, the aromatic amines such as an indolinone-amine, a purine-amine, a pyrimidine-amine and an indole-amine are linked to C(═Y₁) of Formula (I).

In some aspects, the compounds described herein employ tyrosine kinase inhibitors based on an indolinone. In this aspect, the terms “2-indolinone”, “indolin-2-one”, and “2-oxindole” are used interchangeably.

Some of indolinone-containing tyrosine kinase inhibitors contemplated within the present invention have a five-membered heteroaryl ring group (e.g., a pyrrole) or a six-membered aryl ring group (e.g., phenyl) at the 3-position of the indolinone. For example, general structures of certain tyrosine kinase inhibitors based on an indolinone and analogs have the core structure:

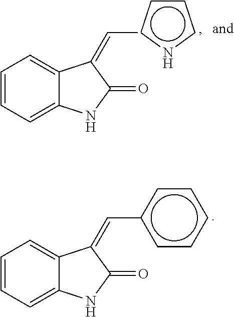

From these core structures, several analogs have been prepared.

According to the present invention, one embodiment can employ an indolinone-containing tyrosine kinase inhibitor having the formula:

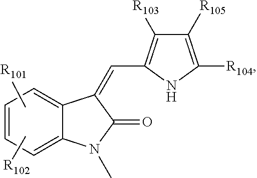

wherein

R₁₀₁ and R₁₀₂ are independently selected from among hydrogen, halogen, alkyls, alkylthio, nitro, trihalomethyl, hydroxy, hydroxyalkyls, alkoxys, cyano, aryl, —C(O)R₁₁, NR₁₂R₁₃, —NR₁₂C(O)R₁₃, —SO₂R₁₂, and —S(O)₂NR₁₂R₁₃,
wherein R₁₁ is selected from among alkyls, amino, hydroxy, alkoxys, aryl, aryloxy, and aminoalkylamino; and R₁₂ and R₁₃ are independently selected from among hydrogen, alkyls, and aryl;

R₁₀₃ is selected from among hydrogen, alkyls (preferably, methyl), hydroxyalkyls, aminoalkyls, —C(O)R₁₁, and aryl;

R₁₀₄ is selected from among hydrogen, alkyls (preferably, methyl), —C(O)R₁₁, and aryl; and

R₁₀₅ is selected from among hydrogen, —CH₂CH₂COOH, —COR₁₄, and —CH₂CH₂C(O)NR₁₅R₁₆, wherein
(a) when R₅ is —COR₁₄, R₁₄ is selected from among alkyls, alkoxys, hydroxy, aryl, aryloxy, alkylamino, dialkylamino, and —NR₃₁R₃₂,
   wherein R₃₁ is hydrogen or an alkyl; and R₃₂ is selected from among aminoalkyls, hydroxyalkyls, acetylalkyls, cyanoalkyls, carboxyalkyls, and alkoxycarbonylalkyls; and wherein the alkyl in the aminoalkyls is optionally substituted with one or two hydroxyl group(s); and
(b) when R₅ is —CH₂CH₂C(O)NR₁₅R₁₆,
   (1) R₁₅ is hydrogen or a C₁₋₄ alkyl; and R₁₆ is -A₁-NR₃₃R₃₄,
      wherein R₃₃ and R₃₄ are independently hydrogen or C₁₋₄ alkyls; and A₁ is (CH₂)ₐ₁, (CH₂)ₐ₂-A₂-(CH₂)ₐ₃ or (CH₂CH₂O)ₐ₄—CH₂CH₂, wherein (a1) is an integer of from about 2 to about 10 (e.g, 2, 3, 4, 5, 6, 7, 8); (a2) and (a3) are independently selected integers of from about 1 to about 6 (e.g., 1, 2, 3, 4, 5, 6); A₂ is CH═CH, phenylene, biphenylene, cyclohexylene or piperazinylene; and (a4) is 1, 2 or 3; or
   (ii) R₁₅ and R₁₆ together form -A₃-NR₃₅-A₄-,
      wherein R₃₅ is hydrogen or C₁₋₄ alkyl; and A₃ and A₄ are independently (CH₂)ₐ₅ or (CH₂CH₂O)ₐ₆CH₂CH₂, wherein (a5) is an integer of from about 2 to about 6 (e.g., 2, 3, 4, 5, 6); and (a6) is 1, 2 or 3; or
   (iii) R₁₅ and R₁₆ together with the nitrogen atom to which they are attached form a piperidinyl, wherein the piperidinyl group bears a substituent of formula -A₅-R₃₆ at the 4 position, wherein A₅ is C₁₋₄ alkylene; and R₃₆ is piperidin-4-yl; or
   (iv) R₁₅ and R₁₆ together with the nitrogen atom to which they are attached form pyrrolidinyl, piperidinyl or morpholino; or

R₁₀₄ and R₁₀₅ together form —(CH₂)₄— or —(CH₂)ₐ₇CO(CH₂)ₐ₈—, wherein (a7) is 0, 1, 2, or 3; (a8) is 0, 1, 2, or 3, provided that the sum of (a7) and (a8) is 3.

In some preferred embodiments, the indolinone-based tyrosine kinase inhibitors are provided in which R₁₀₁ and R₁₀₂ are independently hydrogen, methyl, or ethyl; R₁₀₃ and R₁₀₄ are both methyl; and R₁₀₅ is hydrogen, or —CH₂CH₂COOH.

A representative list of the indolinone-containing biologically active agent includes:

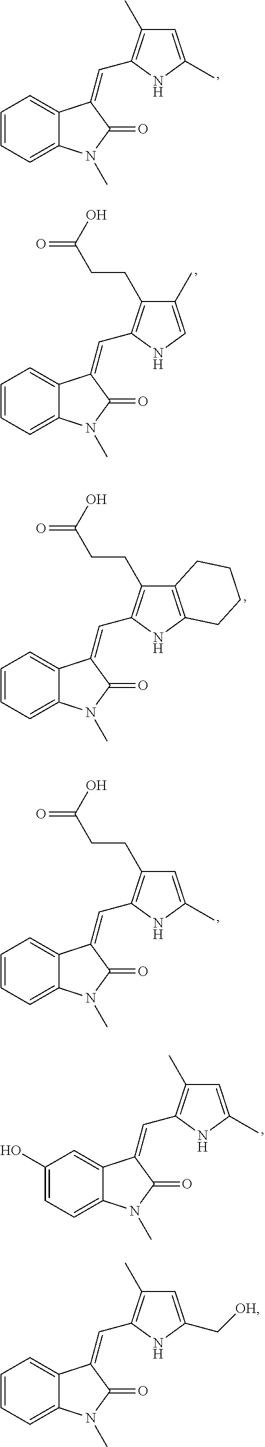

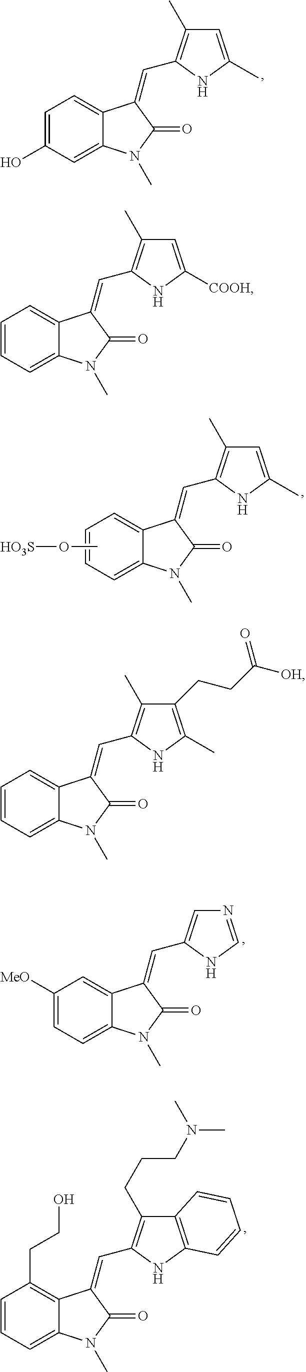

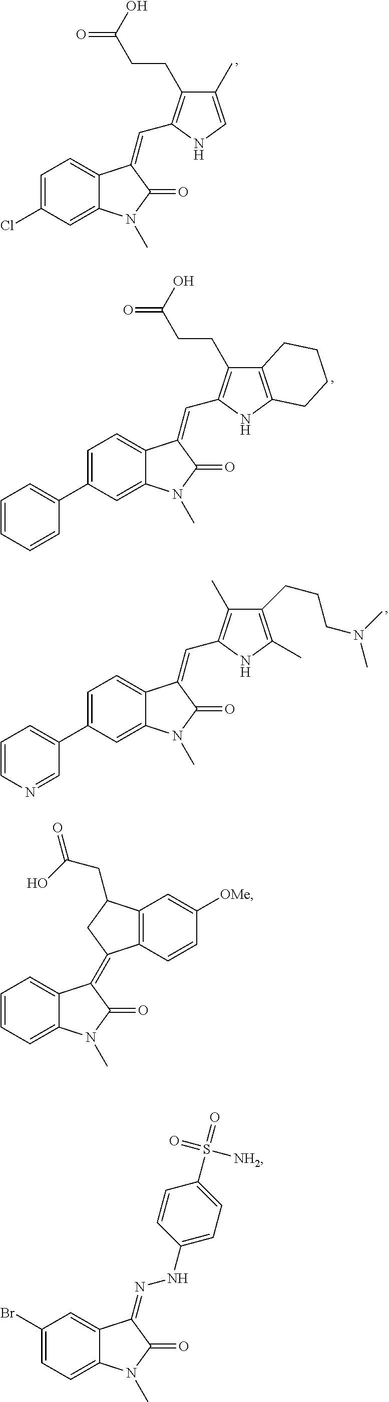

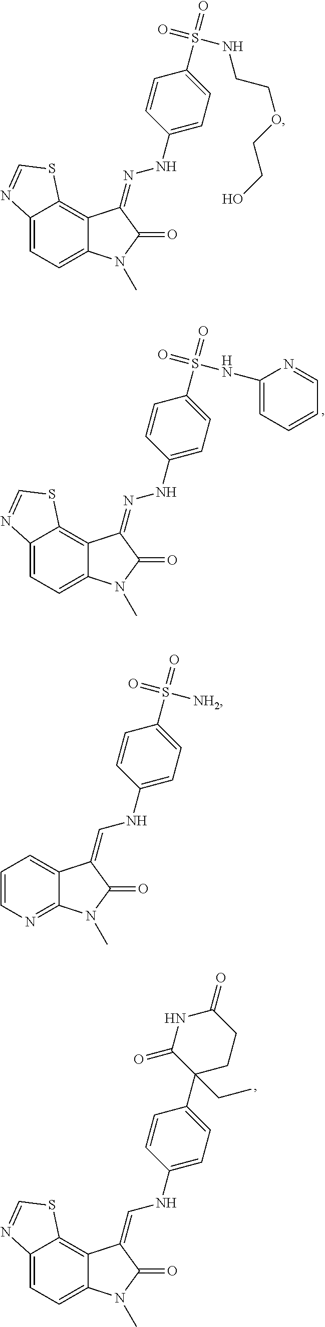

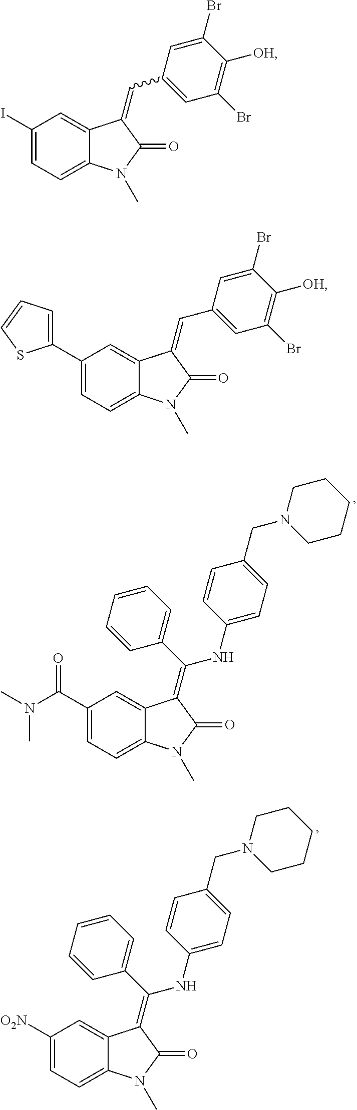

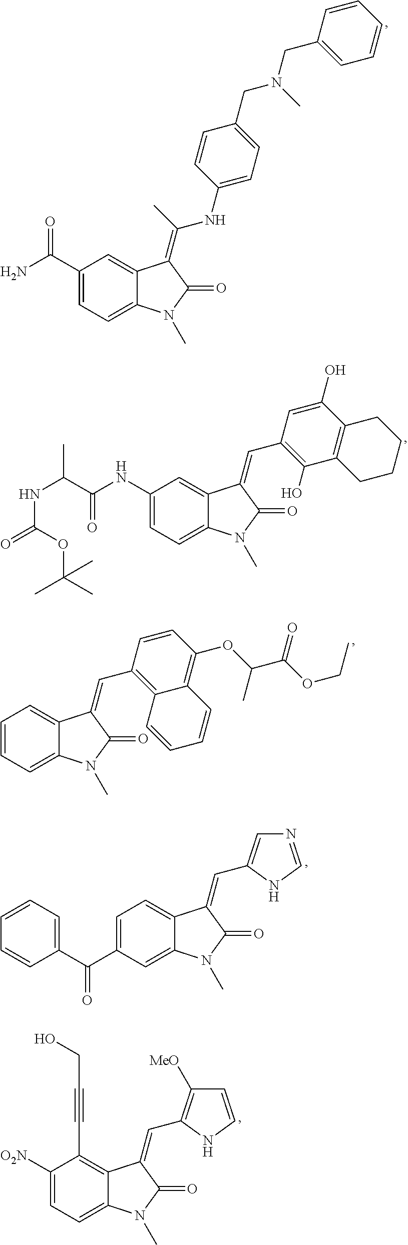

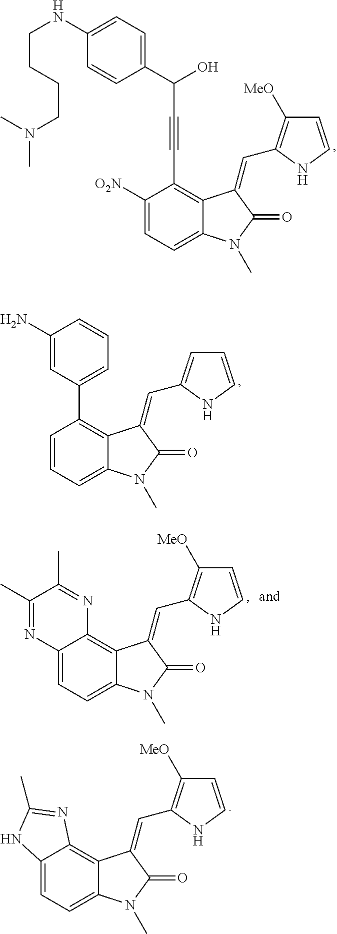

Artisans will appreciate that other substitutions are possible at the 3- and/or 5-position of the indolinone. Additional details of tyrosine kinase inhibitors, including indolinone-based tyrosine kinase inhibitors and analogs, are described in, for example, U.S. Pat. Nos. 5,792,783, 5,834,504, 5,883,113, 5,883,116, 5,886,020, 6,686,362, 6,797,725, 6,927,293, 7,053,114, 7,060,703, 7,186,723, 7,223,783, the contents of each of which are incorporated herein by reference. See also Connell, Expert Opin. Ther. Patents, 2003, 13:737-749; Deprimo et al., 2003, BMC Cancer, 3:3; Itokawa, et al., 2002, Molecular Cancer Therapeutics, 1:295-302; Fiedler et al., 20003, Blood, 102:2763-2767; and Mendel et al., 2000, Clinical Cancer Research, 6:4848-4858, the contents of each of which are incorporated herein by reference. Other useful indolinone-based tyrosine kinase inhibitors are also disclosed in Sun et al., J. Med. Chem. 2000, 43:2655-2663; Antonian et al., 2000, 28:1505-1512; Dumas J, Exp. Opin. Ther. Patents, 2001, 11: 405-429, the contents of each of which are incorporated herein by reference.

The phenyl or pyrrole substituted 2-indolinone derivatives are receptor tyrosine kinase inhibitors useful in the treatment of conditions responsive to receptor tyrosine kinase inhibitors, for example, proliferative disorders such as cancer. The compounds are capable of regulating and/or modulating tyrosine kinase signal transduction including KDR/FLK-1 receptor signal transduction. The compounds can regulate, modulate and/or inhibit vasculogenesis and/or angiogenesis. Indolinone-based tyrosine kinase inhibitors and related analogs are potential anticancer or antitumor agents. However, many of the indolinone analog compounds are insoluble in aqueous solutions and have poor bioavailability.

In another embodiment, the biologically active agent is an indole-containing compound. Some preferred compounds include, without limitation, CDK inhibitors such as paullone. The paullone structures are shown below

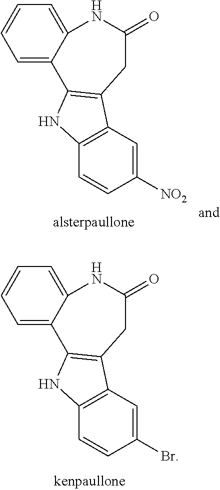

Other known derivatives of the paullone family such as those found in Leost et al., 2000, Eur. J. Biochem, 267:5983-5994, and WO 99/65910, are contemplated as suitable compounds. The disclosures of each of the foregoing are incorporated by reference herein. See also U.S. Pat. No. 7,393,953, the contents of which are incorporated by reference.

In addition, other compounds contemplated as suitable for the present invention include those having the general structures shown below:

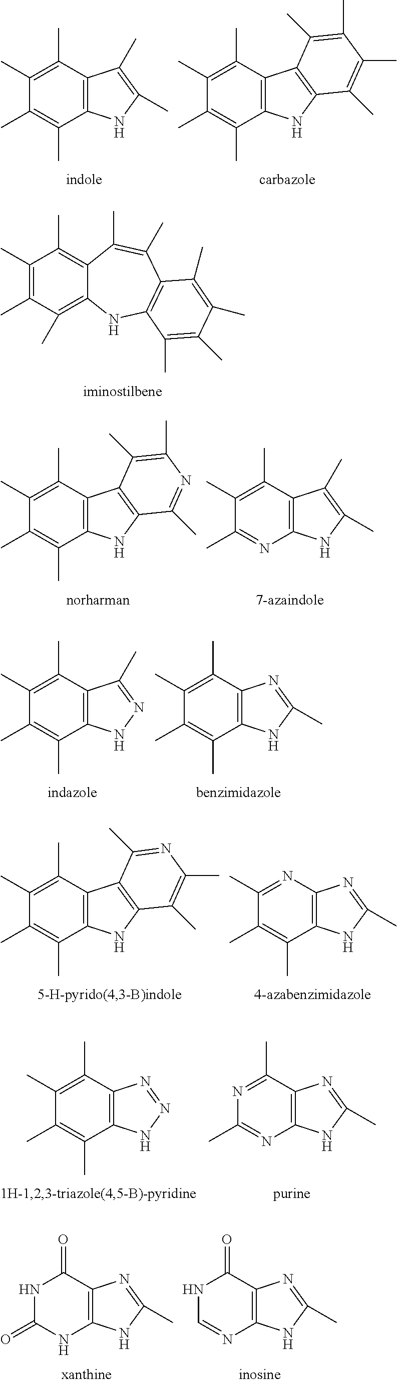

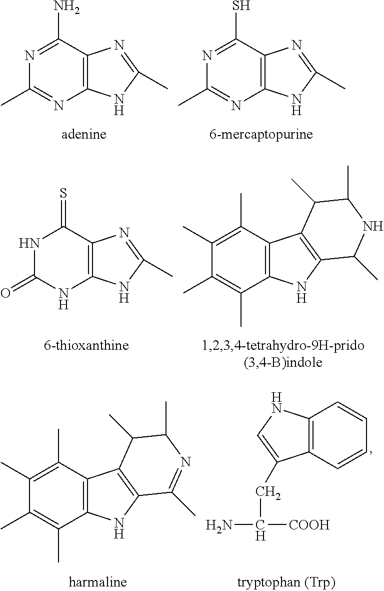

wherein the straight lines indicate possible points of substitution.

Examples of biologically active compounds containing indole or indole-like moieties include, without limitation:
anticancer agents such as

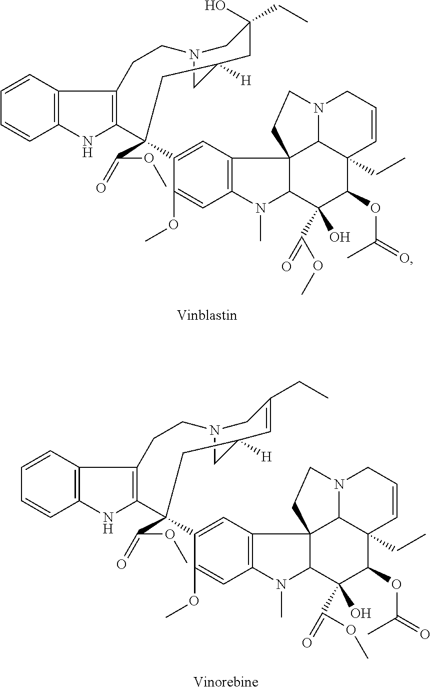

vasodilator, β-adrenergic blocking agents such as

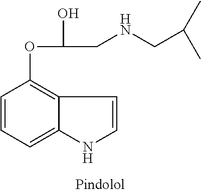

α2 adrenergic antagonists such as

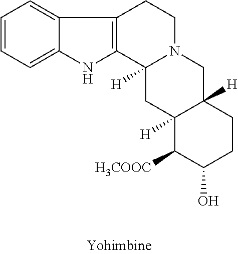

mixed dopamine agonists/antagonists such as

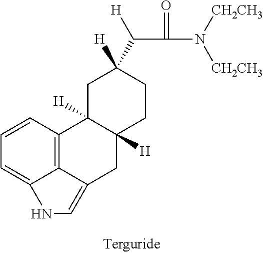

calcium channel blockers such as

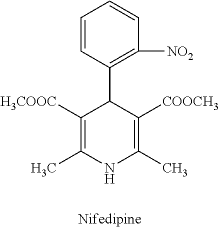

broad range serotonergic, dopaminergic and α-adrenergic active compounds such as

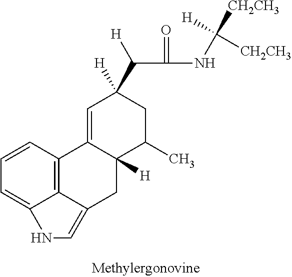

serotonin precursors, antidepressants such as

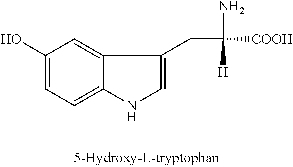

potent 5-HT1c serotonin receptor antagonists such as

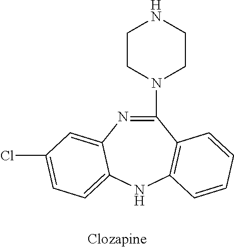

highly selective, non-peptide δ-opioid antagonists such as

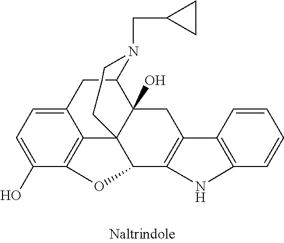

antihypertensive agents such as

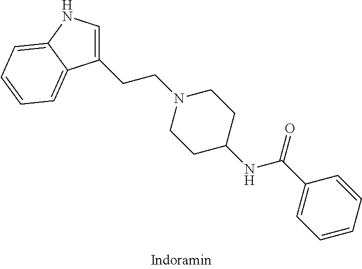

plant growth regulating agents such as

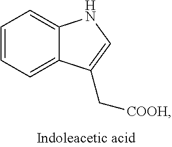

highly selective α-opioid antagonists such as

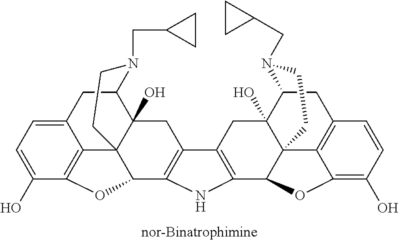

and others selected from anthracycline compounds and related anti-metabolite compounds.

For ease of description and not limitation, the description refers to SU5416 (Semaxanib) as the indolinone-based tyrosine kinase and as the preferred and illustrated compound. It will be understood that the claimed invention includes all other derivatives and analogs so long as the compound has an aromatic amine for the attachment to the releasable urea linker or has an aromatic amine group on the indolinone for the point of attachment to the polymer via a linker. The terms “2-indolinone”, “indolin-2-one”, and “2-oxindole” are used interchangeably. The terms “2-indolinone”, “indolin-2-one”, and “2-oxindole” are used interchangeably.

### C. Bifunctional Linkers (L): L₁, L₂ & L₃

According to present invention, the bifunctional linking moiety, L, described as L, L₁, L₂ or L₃, as included in the compounds provided herein, includes:

—[C(═O)]ᵥ(Y₂₁)ᵤ₁(CR₂₁R₂₂)ₜ₁(Y₂₂)ᵤ₂—,

—[C(═O)]ᵥ(Y₂₁)ᵤ₁(CR₂₃R₂₄O)ₜ₃—,

—[C(═O)]ᵥ(Y₂₁)ᵤ₁(CR₂₃R₂₄O)ₜ₃(CR₂₁R₂₂)ₜ₁(Y₂₂)ᵤ₂—,

—[C(═O)]ᵥ(Y₂₁)ᵤ₁(CR₂₁R₂₂)ₜ₁(CR₂₃R₂₄O)ₜ₃—,

—[C(═O)]ᵥ(Y₂₁)ᵤ₁(CR₂₁R₂₂)ₜ₁Y₂₃(CR₂₁R₂₂)ₜ₂—,

—[C(═O)]ᵥ(Y₂₁)ᵤ₁(CR₂₅R₂₆CR₂₇R₂₈O)ₜ₄—,

—[C(═O)]ᵥ(Y₂₁)ᵤ₁(CR₂₅R₂₆CR₂₇R₂₈O)ₜ₄(CR₂₁R₂₂)ₜ₁(Y₂₂)ᵤ₂—,

—[C(═O)]ᵥ(Y₂₁)ᵤ₁(CR₂₁R₂₂)ₜ₁(CR₂₅R₂₆CR₂₇R₂₈O)ₜ₄—,

—[C(═O)]ᵥ(Y₂₁)ᵤ₁(CR₂₁R₂₂)ₜ₁Y₂₃(CR₂₅R₂₆CR₂₇R₂₈O)ₜ₄—,

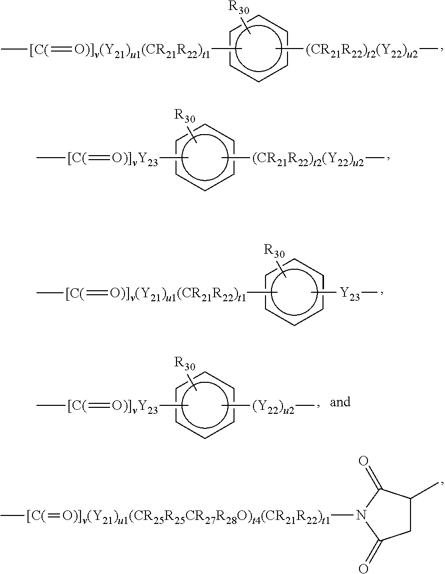

preferably linked to S-containing moiety such as PEG-SH,

wherein

R₂₁-R₃₀ are independently selected from among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy, preferably hydrogen, hydroxyl, amine, and alkyl;

Y₂₁ is O, S or NR₂₉;

Y₂₂ and Y₂₃ are independently O, S or NR₂₉;

(t1) and (t2) are independently positive integers, preferably an integer of from about 1 to about 10 (e.g., 1, 2, 3, 4, 5, 6);

(t3) is a positive integer, preferably an integer of from about 1 to about 10 (e.g., 1, 2, 3, 4, 5, 6);

(t4) is a positive integer, preferably an integer of from about 1 to about 6, (e.g, 1, 2, 3, 4, 5, 6);

(u1) and (u2) are independently zero or 1; and

(v) is zero or 1,

provided that (v) is zero in the first L₁ adjacent to C(═Y₂), when (e1) is a positive integer.

C(R₂₁)(R₂₂), in each occurrence, is the same or different, when (t1) or (t2) is equal to or greater than 2.

C(R₂₃)(R₂₄)O, in each occurrence, is the same or different, when (t3) is equal to or greater than 2.

C(R₂₅)(R₂₆)C(R₂₇)(R₂₈)O, in each occurrence, is the same or different, when (t4) is equal to or greater than 2.

In certain embodiments, L₁ as included in the compounds described herein can be selected from among:

—[C(═O)]ᵥ(Y₂₁)ᵤ₁(CR₂₁R₂₂)ₜ₁(Y₂₂)ᵤ₂—, wherein (t1) is not an integer of from about 1 to about 4, when Y₂₁ and Y₂₂ are both NR₂₉, and (u1) and (u2) are both one;

—[C(═O)]ᵥ(Y₂₁)ᵤ₁(CR₂₃R₂₄O)ₜ₃(CR₂₁R₂₂)ₜ₁(Y₂₂)ᵤ₂—, wherein (t1) and (t3) are not both one, when Y₂₁ and Y₂₂ are both NR₂₉, and (u1) and (u2) are both one;

—[C(═O)]ᵥ(Y₂₁)ᵤ₁(CR₂₁R₂₂)ₜ₁Y₂₃(CR₂₁R₂₂)ₜ₂—, wherein (t1) is not an integer of from 1 to 4, when Y₂₁ and Y₂₃ are both NR₂₉;

—[C(═O)]ᵥ(Y₂₁)ᵤ₁(CR₂₅R₂₆CR₂₇R₂₈O)ₜ₄(CR₂₁R₂₂)ₜ₁(Y₂₂)ᵤ₂—, wherein (t1) and (t4) are not both one, when Y₂₁ and Y₂₂ are both NR₂₉, and (u1) and (u2) are both one;

—[C(═O)]ᵥ(Y₂₁)ᵤ₁(CR₂₁R₂₂)ₜ₁Y₂₃(CR₂₅R₂₆CR₂₇R₂₈O)ₜ₄—, wherein (t1) is not an integer of from 1 to 4, when Y₂₁ and Y₂₃ are both NR₂₉, and (u1) is one;

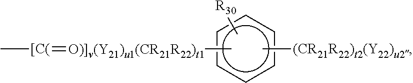

wherein R₃₀ in the ortho position relative to [C(═O)]ᵥY₂₁(CR₂₁R₂₂)ₜ₁ is not NHR₄₁ or CR₄₂R₄₃NHR₄₁, when Y₂₁ is NR₂₉, and (u1) and (t1) are both one; and wherein R₃₀ in the ortho position relative to [C(═O)]ᵥY₂₁(CR₂₁R₂₂)ₜ₁ is not NHR₄₁, when Y₂₁ is NR₂₉, (u1) is one, and (t1) is two;

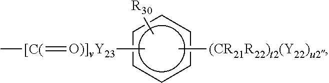

wherein R₃₀ in the ortho position relative to [C(═O)]ᵥY₂₃ is not NHR₄₁, CR₄₂R₄₃NHR₄₁ or (CR₄₂R₄₃)₂NHR₄₁, when Y₂₃ is NR₂₉;

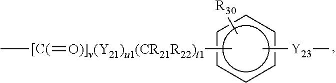

wherein R₃₀ in the ortho position relative to [C(═O)]ᵥY₂₁(CR₂₁R₂₂)ₜ₁ is not NHR₄₁ or CR₄₂R₄₃NHR₄₁, when Y₂₂ is NR₂₉, and (u1) and (t1) are both one; and wherein R₃₀ in the ortho position relative to [C(═O)]ᵥY₂₁(CR₂₁R₂₂)ₜ₁ is not NHR₄₁, when Y₂₁ is NR₂₉, (u1) is one, and (t1) is two; and

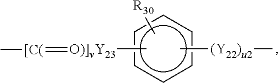

wherein R₃₀ in the ortho position relative to [C(═O)]ᵥY₂₃ is not NHR₄₁, CR₄₂R₄₃NHR₄₁ or (CR₄₂R₄₃)₂NHR₄₁, when Y₂₃ is NR₂₉,

wherein R₄₁-R₄₃ are independently hydrogen or alkyls.

Suitable L₁ groups as included in the compounds described herein can be selected among:

—[C(═O)]ᵥ(CR₂₁R₂₂)ₜ₁—,

—[C(═O)]ᵥ(CR₂₂R₂₃)ₜ₁O—,

—[C(═O)]ᵥ(CR₂₂R₂₃)ₜ₁NR₂₉—,

—[C(═O)]ᵥO(CR₂₁R₂₂)ₜ₁—,

—[C(═O)]ᵥO(CR₂₁R₂₂)ₜ₁O—,

—[C(═O)]ᵥO(CR₂₁R₂₂)ₜ₁NR₂₉—,

—[C(═O)]ᵥNR₂₉(CR₂₂R₂₃)ₜ₁—,

—[C(═O)]ᵥNR₂₉(CR₂₂R₂₃)ₜ₁O—,

—[C(═O)]ᵥNR₂₉(CR₂₂R₂₃)ₜ₁NR₂₉—,

—[C(═O)]ᵥS(CR₂₁R₂₂)ₜ₁—,

—[C(═O)]ᵥS(CR₂₁R₂₂)ₜ₁O—,

—[C(═O)]ᵥS(CR₂₁R₂₂)ₜ₁NR₂₉—,

—[C(═O)]ᵥ(CR₂₃R₂₄O)ₜ₁—,

—[C(═O)]ᵥO(CR₂₃R₂₄O)ₜ₁—,

—[C(═O)]ᵥNR₂₉(CR₂₃R₂₄O)ₜ₁—,

—[C(═O)]ᵥ(CR₂₃R₂₄O)ₜ₃(CR₂₁R₂₂)ₜ₁—,

—[C(═O)]ᵥO(CR₂₃R₂₄O)ₜ₃(CR₂₁R₂₁)ₜ₁—,

—[C(═O)]ᵥNR₂₉(CR₂₃R₂₄O)ₜ₃(CR₂₁R₂₂)ₜ₁—,

—[C(═O)]ᵥ(CR₂₃R₂₄O)ₜ₃(CR₂₁R₂₂)ₜ₁O—,

—[C(═O)]ᵥ(CR₂₃R₂₄O)ₜ₃(CR₂₁R₂₂)ₜ₁NR₂₉—,

—[C(═O)]ᵥO(CR₂₃R₂₄O)ₜ₃(CR₂₁R₂₂)ₜ₁O—,

—[C(═O)]ᵥO(CR₂₃R₂₄O)ₜ₃(CR₂₁R₂₂)ₜ₁NR₂₉—,

—[C(═O)]ᵥNR₂₉(CR₂₃R₂₄O)ₜ₃(CR₂₁R₂₂)ₜ₁O—,

—[C(═O)]ᵥNR₂₉(CR₂₃R₂₄O)ₜ₃(CR₂₁R₂₂)ₜ₁NR₂₉—,

—[C(═O)]ᵥ(CR₂₁R₂₂)ₜ₁(CR₂₃R₂₄O)ₜ₁—,

—[C(═O)]ᵥO(CR₂₁R₂₂)ₜ₁(CR₂₃R₂₄O)ₜ₁—,

—[C(═O)]ᵥNR₂₉(CR₂₁R₂₂)ₜ₁(CR₂₃R₂₄O)ₜ₃—,

—[C(═O)]ᵥ(CR₂₁R₂₂)ₜ₁O(CR₂₁R₂₂)ₜ₂—,

—[C(═O)]ᵥ(CR₂₁R₂₂)ₜ₁NR₂₉(CR₂₁R₂₂)ₜ₂—,

—[C(═O)]ᵥ(CR₂₁R₂₂)ₜ₁S(CR₂₁R₂₂)ₜ₂—,

—[C(═O)]ᵥO(CR₂₁R₂₂)ₜ₁(CR₂₁R₂₂)ₜ₂—,

—[C(═O)]ᵥO(CR₂₁R₂₂)ₜ₁NR₂₉(CR₂₁R₂₂)ₜ₂—,

—[C(═O)]ᵥO(CR₂₁R₂₂)ₜ₁S(CR₂₁R₂₂)ₜ₂—,

—[C(═O)]ᵥNR₂₉(CR₂₁R₂₂)ₜ₁O(CR₂₁R₂₂)ₜ₂—,

—[C(═O)]ᵥNR₂₉(CR₂₁R₂₂)ₜ₁NR₂₉ (CR₂₁R₂₂)ₜ₂—,

—[C(═O)]ᵥNR₂₉(CR₂₁R₂₂)ₜ₁S(CR₂₁R₂₂)ₜ₂—,

—[C(═O)]ᵥ(CR₂₅R₂₆CR₂₇R₂₈O)ₜ₄—,

—[C(═O)]ᵥO(CR₂₅R₂₆CR₂₇R₂₈O)ₜ₄—,

—[C(═O)]ᵥNR₂₉(CR₂₅R₂₆CR₂₇R₂₈O)ₜ₄—,

—[C(═O)]ᵥ(CR₂₅R₂₆CR₂₇R₂₈O)ₜ₄(CR₂₁R₂₂)ₜ₁—,

—[C(═O)]ᵥ(CR₂₅R₂₆CR₂₇R₂₈O)ₜ₄(CR₂₁R₂₂)ₜ₁O—,

—[C(═O)]ᵥ(CR₂₅R₂₆CR₂₇R₂₈O)ₜ₄(CR₂₁R₂₂)ₜ₁NR₂₉—,

—[C(═O)]ᵥO(CR₂₅R₂₆CR₂₇R₂₈O)ₜ₄(CR₂₁R₂₂)ₜ₁—,

—[C(═O)]ᵥO(CR₂₅R₂₆CR₂₇R₂₈O)ₜ₄(CR₂₁R₂₂)ₜ₁O—,

—[C(═O)]ᵥO(CR₂₅R₂₆CR₂₇R₂₈O)ₜ₄(CR₂₁R₂₂)ₜ₁NR₂₉—,

—[C(═O)]ᵥNR₂₉(CR₂₅R₂₆CR₂₇R₂₈O)ₜ₄(CR₂₁R₂₂)ₜ₁—,

—[C(═O)]ᵥNR₂₉(CR₂₅R₂₆CR₂₇R₂₈O)ₜ₁(CR₂₁R₂₂)ₜ₁O—,

—[C(═O)]ᵥNR₂₉(CR₂₅R₂₆CR₂₇R₂₈O)ₜ₄(CR₂₁R₂₂)ₜ₁NR₂₉—,

—[C(═O)]ᵥ(CR₂₁R₂₂)ₜ₁(CR₂₅R₂₆CR₂₇R₂₈O)ₜ₄—,

—[C(═O)]ᵥO(CR₂₁R₂₂)ₜ₁(CR₂₅R₂₆CR₂₇R₂₈O)ₜ₄—,

—[C(═O)]ᵥNR₂₉(CR₂₁R₂₂)ₜ₁(CR₂₅R₂₆CR₂₇R₂₈O)ₜ₄—,

—[C(═O)]ᵥ(CR₂₁R₂₂)ₜ₁O(CR₂₅R₂₆CR₂₇R₂₈O)ₜ₄—,

—[C(═O)]ᵥ(CR₂₁R₂₂)ₜ₁S(CR₂₅R₂₆CR₂₇R₂₈O)ₜ₄—,

—[C(═O)]ᵥ(CR₂₁R₂₂)ₜ₁NR₂₉(CR₂₅R₂₆CR₂₇R₂₈O)ₜ₄—,

—[C(═O)]ᵥO(CR₂₁R₂₂)ₜ₁O(CR₂₅R₂₆CR₂₇R₂₈O)ₜ₄—,

—[C(═O)]ᵥO(CR₂₁R₂₂)ₜ₁S(CR₂₅R₂₆CR₂₇R₂₈O)ₜ₄—,

—[C(═O)]ᵥO(CR₂₁R₂₂)ₜ₁NR₂₉(CR₂₅R₂₆CR₂₇R₂₈O)ₜ₄—,

—[C(═O)]ᵥNR₂₉(CR₂₁R₂₂)ₜ₁O(CR₂₅R₂₆CR₂₇R₂₈O)ₜ₄—,

—[C(═O)]ᵥNR₂₉(CR₂₁R₂₂)ₜ₁S(CR₂₅R₂₆CR₂₇R₂₈O)ₜ₄—,

—[C(═O)]ᵥNR₂₉(CR₂₁R₂₂)ₜ₁NR₂₉(CR₂₅R₂₆CR₂₇R₂₈O)ₜ₄—,

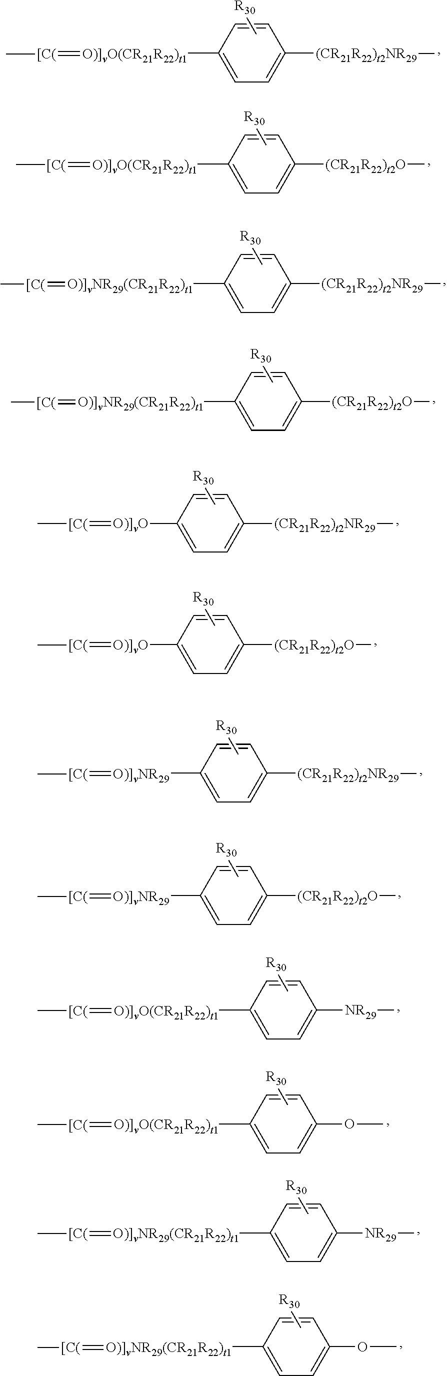

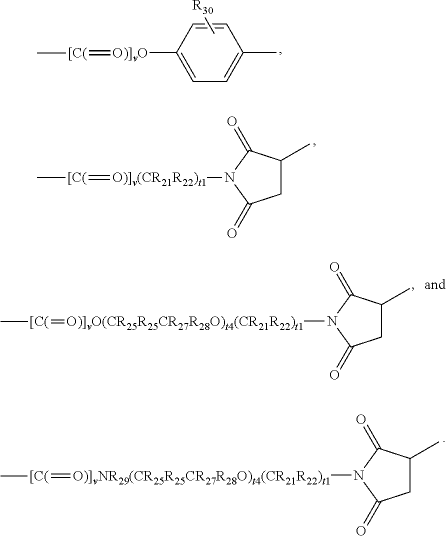

In certain embodiments, the L₁ groups can be selected from among:

—[C(═O)]ᵥNR₂₉(CR₂₂R₂₃)ₜ₁NR₂₉—, wherein (t1) is not an integer of from 1 to 4;

—[C(═O)]ᵥNR₂₉(CR₂₃R₂₄O)ₜ₃(CR₂₁R₂₂)ₜ₁NR₂₉—, wherein (t1) and (t3) are not both one;

—[C(═O)]ᵥNR₂₉(CR₂₁R₂₂)ₜ₁NR₂₉(CR₂₁R₂₂)ₜ₂—, wherein (t1) is not an integer of from 1 to 4;

—[C(═O)]ᵥNR₂₉(CR₂₅R₂₆CR₂₇R₂₈O)ₜ₄(CR₂₁R₂₂)ₜ₁NR₂₉—, wherein (t1) and (t4) are not both one;

—[C(═O)]ᵥNR₂₉(CR₂₁R₂₂)ₜ₁NR₂₉(CR₂₅R₂₆CR₂₇R₂₈O)ₜ₄—, wherein (t) is not an integer of from 1 to 4;

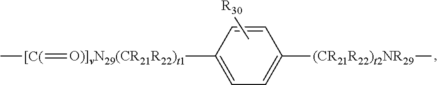

wherein R₃₀ in the ortho position relative to [C(═O)]ᵥNR₂₉(CR₂₁R₂₂)ₜ₁ is not NHR₄₁ or CR₄₂R₄₃NHR₄₁, when (t1) is one; R₃₀ in the ortho position relative to [C(═O)]ᵥNR₂₉(CR₂₁R₂₂)ₜ₁ is not NHR₄₁, when (t1) is two;

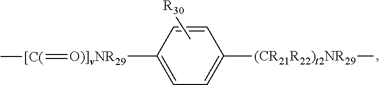

wherein R₃₀ in the ortho position relative to [C(═O)]ᵥNR₂₉ is not NHR₄₁, CR₄₂R₄₃NHR₄₁ or (CR₄₂R₄₃)₂NHR₄₁; and

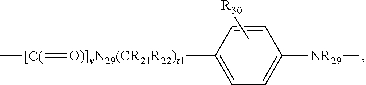

wherein R₃₀ in the ortho position relative to [C(═O)]ᵥNR₂₉(CR₂₁R₂₂)ₜ₁ is not NHR₄₁ or CR₄₂R₄₃NHR₄₁, when (t1) is one; R₃₀ in the ortho position relative to [C(═O)]ᵥNR₂₉(CR₂₁R₂₂)ₜ₁ is not NHR₄₁, when (t1) is two.

In one particular embodiment, C(═Y₂) together with L₁ forms the following, without limitation,

—C(═O)O(CH₂CH₂O)(CH₂)₂NH—,

—C(═O)O(CH₂)₃NH—, and

—C(═O)NH(CH₂CH₂O)₂(CH₂)₂NH—.

In a further and/or alternative aspect of the present invention, the bifunctional linking moiety group (L₂) includes an amino acid. The amino acid which can be selected from any of the known naturally-occurring L-amino acids is, e.g., alanine, valine, leucine, isoleucine, glycine, serine, threonine, methionine, cysteine, phenylalanine, tyrosine, tryptophan, aspartic acid, glutamic acid, lysine, arginine, histidine, proline, and/or a combination thereof, to name a few. In alternative aspects, L can be a peptide residue. The peptide can range in size, for instance, from about 2 to about 10 amino acid residues (e.g., 2, 3, 4, 5, or 6).

Derivatives and analogs of the naturally occurring amino acids, as well as various art-known non-naturally occurring amino acids (D or L), hydrophobic or non-hydrophobic, are also contemplated to be within the scope of the invention. Simply by way of example, amino acid analogs and derivates include:

2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine or sarco sine, N-methylisoleucine, 6-N-methyllysine, N-methylvaline, norvaline, norleucine, ornithine, and others too numerous to mention, that listed in 63 Fed. Reg., 29620, 29622 are incorporated herein by reference. Some preferred L groups include glycine, alanine, methionine or sarcosine.

In certain embodiments, —NT₂-C(═Y₂) together with the first L₂ adjacent to C(═Y₂) forms releasable linking moieties: —NH—C(═O)-Gly-Leu-Phe-Gly(NH)— (SEQ ID NO: 7), —NH—C(═O)-Leu-Ala-Leu-Ala(NH)— (SEQ ID NO: 8), —NH—C(═O)-Lys-Phe(NH)—, or —NH—C(═O)—Cit-Val(NH)—, when T₂ is hydrogen. The amino acids are described in the orientation of from the C-terminal to the N-terminal. The N-terminal is indicated as (NH), when amino acids are described from the C-terminal to the N-terminal.

In another aspect, the bifunctional linker can include a releasable linker L₃. Examples of suitable releasable linkers have the formula:

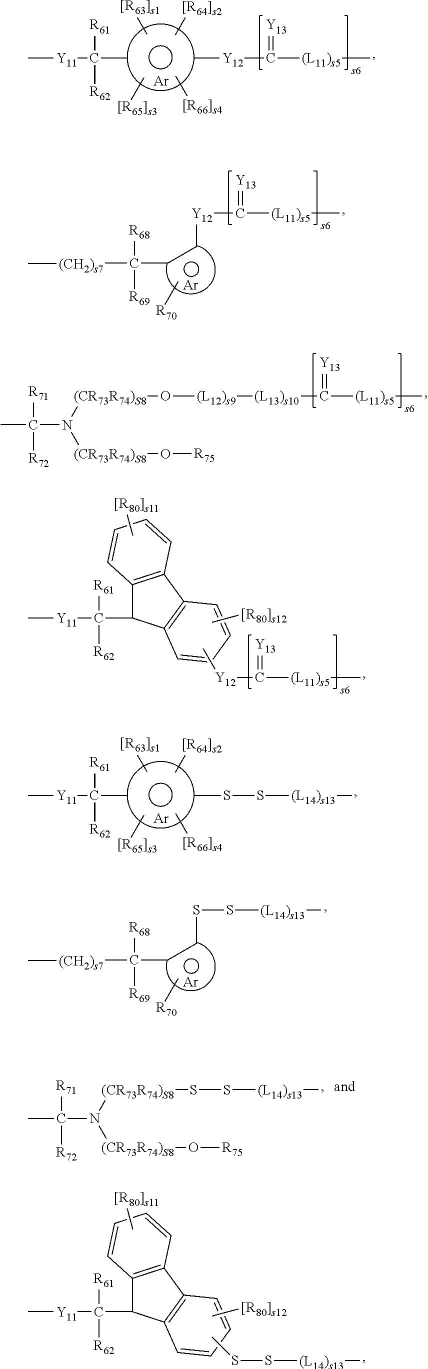

wherein

Y₁₁ is O, or S;

Y₁₂ is O, S, or NH, provided that L₁₁ is Gly-Phe-Leu-Gly (SEQ ID NO: 7), Ala-Leu-Ala-Leu (SEQ ID NO: 8), Phe-Lys, or Val-Cit, when Y₁₂ is NH and (s6) is one;

Y₁₃ is O, S, or NR₆₇;

L₁₁ and L₁₃ are independently bifunctional linking moieties, and the same as defined for L₁ and L₂, (provided that (v) is zero in the first L₁₃, when (s9) is one; (v) is one in the first L₁₃, when (s9) is zero; (v) is zero in the =L₁₁ adjacent to C(═Y₁₃));

L₁₂ is
—C(O)CR₇₆R₇₇OCR₇₆R₇₇C(O)—;
—C(O)CR₇₆R₇₇NR₇₈CR₇₆R₇₇C(O)—;
—C(O)CR₇₆R₇₇SCR₇₆R₇₇C(O)—, or
—C(O)(CR₇₆R₇₇)ₛ₁₁C(O)—;

L₁₄ is a bifunctional linking moiety, and the same as defined as L₁ and L₂, preferably, (CR₂₁R₂₂)₂NH, provided that (v) is zero in the L₁₄ adjacent to S—S;

R₆₁, R₆₂, R₆₇, R₇₁, R₇₂, R₇₃ and R₇₄ are independently selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, preferably hydrogen, and C₁₋₆ alkyls;

R₆₃, R₆₄, R₆₅ and R₆₆ are independently selected from among hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;

R₆₈, R₆₉ and R₇₀ are independently selected from among C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;

R₇₅ is H, —C(═O)—R₇₉, wherein R₇₉, in each occurrence, is the same or different alkyl,

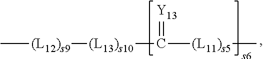

or
a targeting group;

R₇₆, R₇₇ and R₇₈ are independently selected from among from hydrogen, C₁₋₆ alkyl, C₂₋₆ alkenyl, C₂₋₆ alkynyl, C₁₋₆ heteroalkyl and aryl;

R₈₀, in each occurrence, is independently selected from among SO₃H, NO₂, F, Cl, Br, I, CN, C(O)—R₇₉, COOH, COOR₇₉, CHO, COR₇₉, N(R₇₉)₃⁺, CF₃, and CCl₃;

Ar is a moiety which when included in Formula (I) forms an aromatic or heteroaromatic hydrocarbon;

(s1), (s2), (s3), and (s4) are independently zero or one; (s5) is a positive integer, preferably an integer of from about 1 to about 6 (e.g., 1, 2, 3, 4, 5, 6);

(s6) is zero or one;

(s7) is zero, one or two;

(s8) is 1, 2 or 3, preferably 2;

(s9) is zero or one;

(s10) is zero or a positive integer of from 1 to about 6 (e.g., 1, 2, 3, 4, 5, 6);

(s11) and (s12) are independent zero, 1 or 2, and preferably, the sum of (s11) and (s12) is equal to or greater than 1; and

(s13) is a positive integer.

In a further embodiment, the compound of Formula (I) wherein L is L₃, L₁₁ and L₁₃ are independently bifunctional linking moiety, and the same as defined as L₁ and L₂; where (v) is zero in the first L₁₃, when (s9) is one; (v) is one in the first L₁₃, when (s9) is zero.

In this aspect, C(═Y₁) together with L₃ or C(═Y₂) together with L₃-T₃ is selected from among:

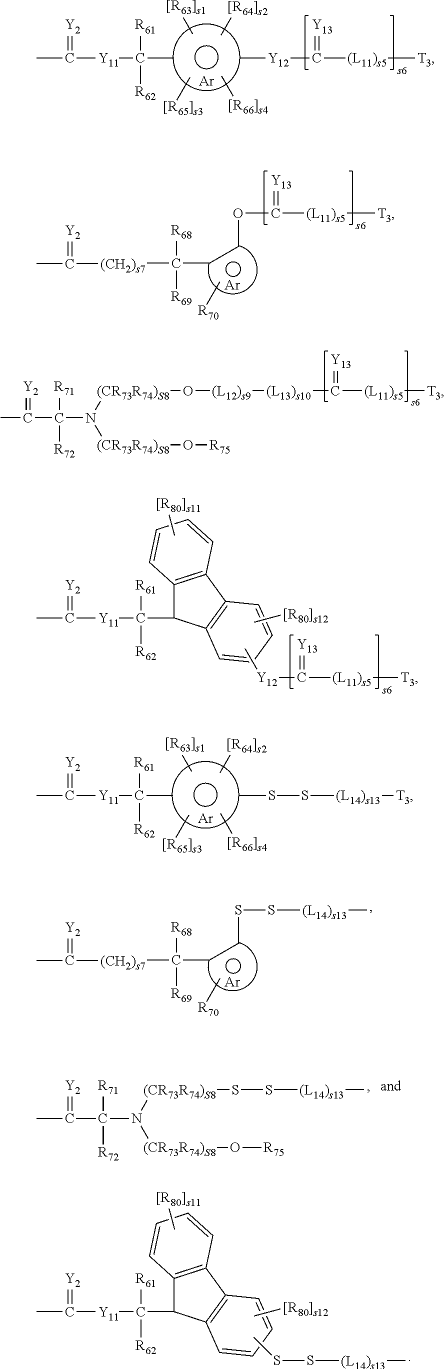

In this aspect, Y₁₂—C(═Y₁₃) together with the L₁₁ adjacent to Y₁₂—C(═Y₁₃) forms —NH—C(═O)-Gly-Leu-Phe-Gly(NH)— (SEQ ID NO: 7), —NH—C(═O)-Leu-Ala-Leu-Ala(NH)—(SEQ ID NO: 8), —NH—C(═O)-Lys-Phe(NH)—, or —NH—C(═O)—Cit-Val(NH)—, when Y₁₂ is NH. The amino acids are shown in the orientation of from the C-terminal to the N-terminal.

Examples of —C(═Y₂)-L₃-T₃ or —C(═Y₁)-[L]ₚ-R are selected from among:

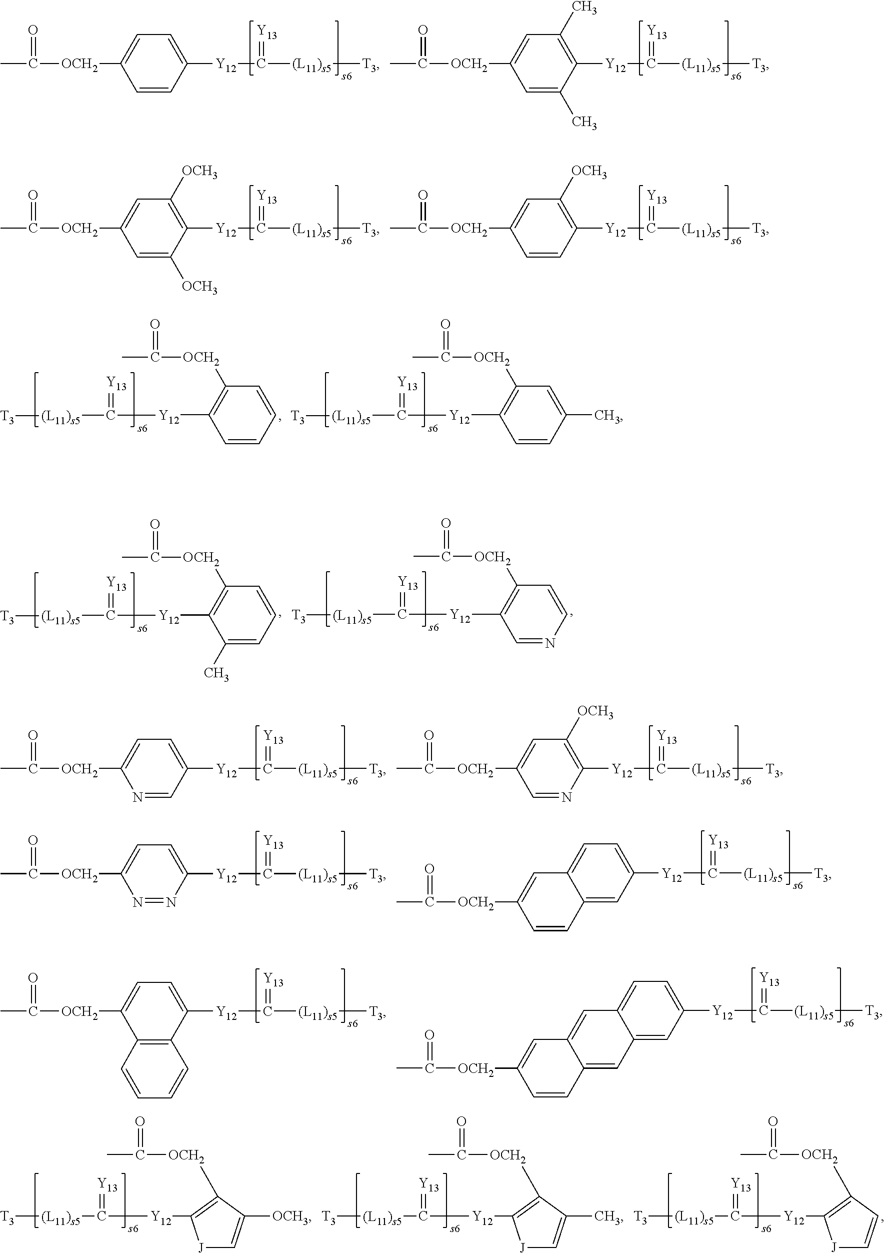

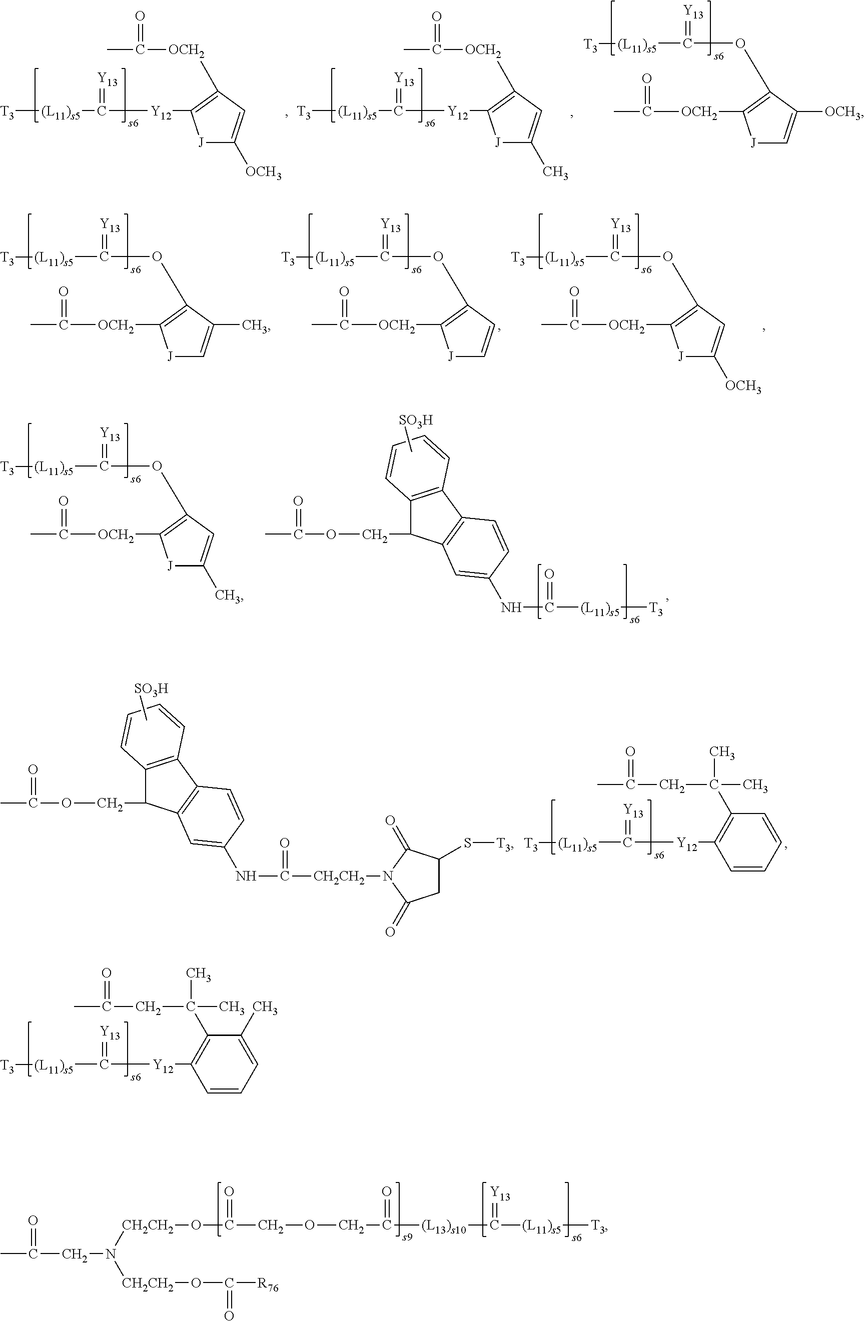

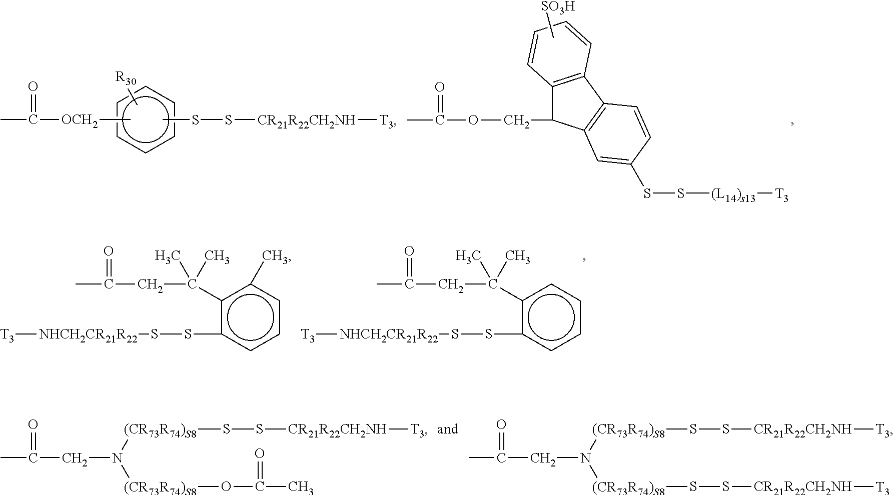

wherein

T₃ is selected from among hydrogen, C₁₋₆ alkyls, C₁₋₆ alkenyls, C₁₋₆ alkynyls, C₃₋₈ cycloalkyls, aryls, leaving groups, functional groups, targeting groups and substantially non-antigenic polymers such as a polyethylene having the structure: —[C(═O)]_{f2}—(CH₂)_{f1}-M₁-CH₂CH₂(OCH₂CH₂)ₙ—;

M₁ is O, or NH;

J is O, S, or NR₈₁;

R₈₁ is selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, and substituted C₁₋₆ heteroalkyls;

(f1) is zero, 1, 2, or 3;

(f2) is zero or 1; and

(n) is a positive integer of from about 10 to about 2,300.

The combinations of the bifunctional linkers, including releasable linkers, contemplated within the scope of the present invention include those in which combinations of variables and substituents of the linker groups are permissible so that such combinations result in stable compounds of Formula (I). In one example, the combinations of values and substituents do not permit a carbonyl group to be positioned directly adjacent to a carbonyl group. In another example, the combinations of values and substituents do not permit oxygen, nitrogen or carbonyl to be positioned directly adjacent to S—S.

In some aspects of the present invention, the compounds of Formula (I) include from 1 to about 6 units (e.g., 1, 2, 3, 4, 5, or 6) of the bifunctional linker. In some preferred aspects of the present invention, the compounds include zero or one unit of the bifunctional linker and thus (e2) is zero or 1.

Additional linkers are found in Table 1 of Greenwald et al. (Bioorganic & Medicinal Chemistry, 1998, 6:551-562), U.S. Pat. Nos. 5,965,119; 6,180,095; 6,214,330; and 6,303,569, the contents of each of which are incorporated herein by reference.

### D. Substantially Non-Antigenic Polymers

A further aspect of the invention provides compounds described herein containing a polymer. Polymers contemplated within the compounds described herein are preferably water soluble and substantially non-antigenic, and include, for example, polyalkylene oxides (PAO's). The compounds described herein further include linear, branched, or multi-armed polyalkylene oxides. In one preferred aspect of the invention, the polyalkylene oxide includes polyethylene glycols and polypropylene glycols. More preferably, the polyalkylene oxide includes polyethylene glycol (PEG).

The polyalkylene oxide has a total number average molecular weight of from about 2,000 to about 100,000 daltons, preferably from about 5,000 to about 60,000 daltons. The polyalkylene oxide can be more preferably from about 5,000 to about 25,000 or from about 20,000 to about 45,000 daltons. In some particularly preferred embodiments, the compounds described herein include the polyalkylene oxide having a total number average molecular weight of from about 30,000 to about 45,000 daltons. In one particular embodiment, a polymeric portion has a total number average molecular weight of about 40,000 daltons.

PEG is generally represented by the structure:
—(CH₂CH₂O)ₙ—

where (n) is a positive integer of from about 10 to about 2300 so that the polymeric portion of the compounds described herein has a number average molecular weight of from about 2,000 to about 100,000 daltons. (n) represents the degree of polymerization for the polymer, and is dependent on the molecular weight of the polymer.

Alternatively, the polyethylene glycol can be represented by the structure:

—[C(═O)]_{f2}—(CH₂)_{f1}-M₁-CH₂CH₂(OCH₂CH₂)ₙ—

wherein

M₁ is O, S, or NH;

(f1) is zero or a positive integer of from about 1 to about 10, preferably, 0, 1, 2, or 3, more preferably, zero or 1;

(f2) is zero or one; and

(n) is a positive integer of from about 10 to about 2,300.

In yet alternative embodiment, the polyethylene glycol (PEG) residue portion can be represented by the structure:

—Y₇₁—(CH₂CH₂O)ₙ—CH₂CH₂Y₇₁—,

—Y₇₁—(CH₂CH₂O)ₙ—CH₂C(═Y₇₂)—Y₇₁—,

—Y₇₁—C(═Y₇₂)—(CH₂)ₐ₁₁—Y₇₃—(CH₂CH₂O)ₙ—CH₂CH₂—Y₇₃—(CH₂)ₐ₁₁—C(═Y₇₂)—Y₇₁— and

—Y₇₁—(CR₇₁R₇₂)ₐ₁₁—Y₇₃—(CH₂)_{b11}—O—(CH₂CH₂O)ₙ—(CH₂)_{b11}—Y₇₃—(CR₇₁R₇₂)ₐ₁₁—Y₇₁—,

wherein:

Y₇₁ and Y₇₃ are independently O, S, SO, SO₂, NR₇₃ or a bond;

Y₇₂ is O, S, or NR₇₄;

R₇₁₋₇₄ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy, preferably hydrogen, methyl, ethyl or propyl;

(a11) and (b11) are independently zero or positive integers, preferably zero or positive integers of from about 1 to about 6 (i.e., 1, 2, 3, 4), and more preferably 1; and

(n) is an integer of from about 10 to about 2300.

The terminal end (A group) of PEG can end with hydrogen, NH₂, OH, CO₂H, C₁₋₆ alkyl (e.g., methyl, ethyl, propyl), C₁₋₆ alkoxy (e.g., methoxy, ethoxy, propyloxy), acyl or aryl. In one embodiment, the terminal hydroxyl group of PEG is substituted with a methoxy or methyl group. In one preferred embodiment, the PEG employed in the compounds described herein is methoxy PEG.

Suitable polymers as included in the compounds of Formula (I) or Formula (I′) correspond to polymer systems (Va)-(Vh) with the following structure:

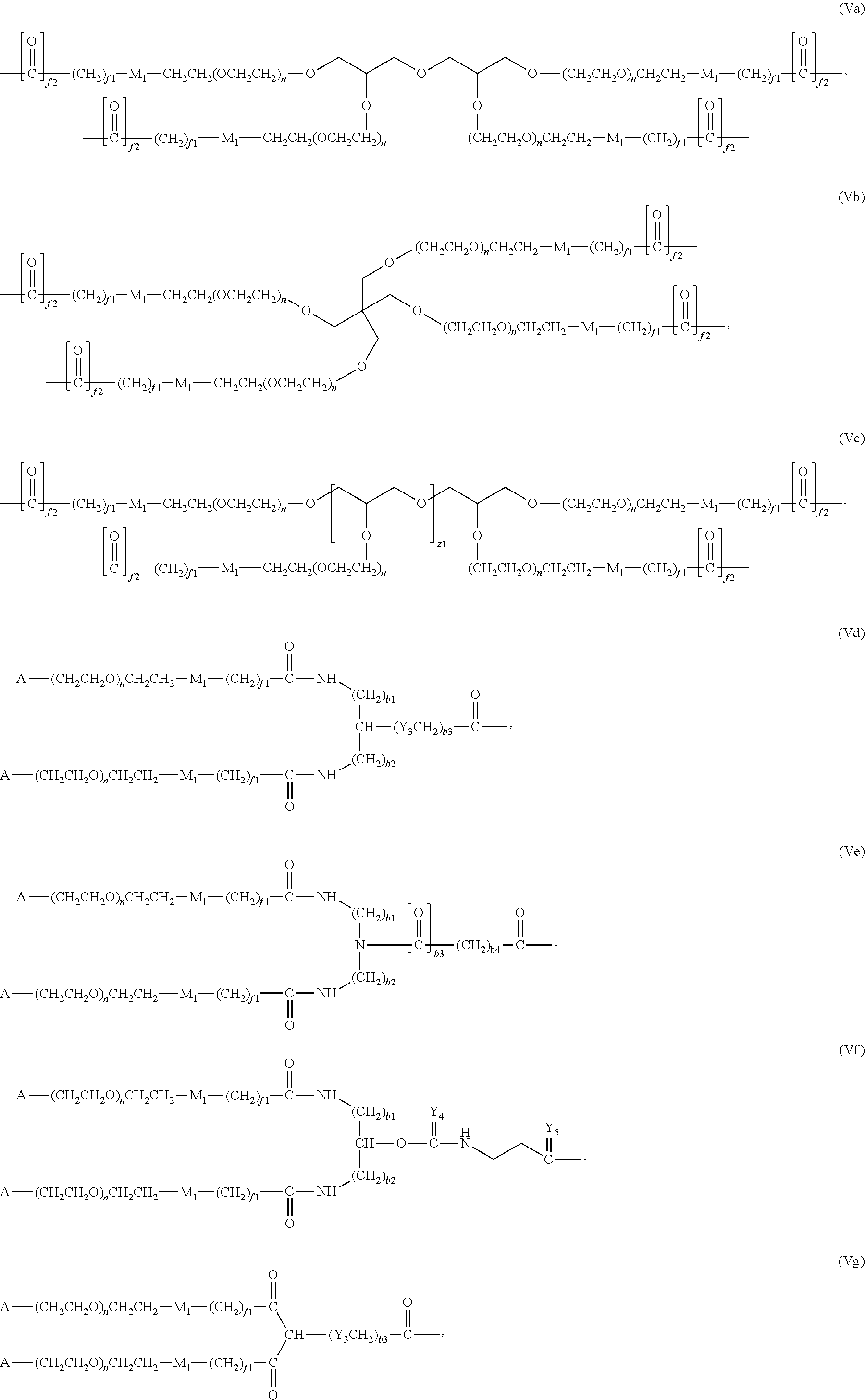

and

—[C(═O)]_{f2}—(CH₂)_{f1}-M₁-CH₂CH₂—O—(CH₂CH₂O)ₙ—CH₂CH₂-M₁-(CH₂)_{f1}—[C(═O)]_{f2}—, (Vh)

wherein

A is hydroxyl, NH₂, CO₂H, or C₁₋₆ alkoxy;

M₁ is O, S, or NH;

Y₃ is O, NR₅₁, S, SO or SO₂;

Y₄ and Y₅ are independently O, S or NR₅₁; and

R₅₁, in each occurrence, is independently hydrogen, C₁₋₈ alkyl, C₁₋₈ branched alkyl, C₁₋₈ substituted alkyl, aryl, or aralkyl.

Branched or U-PEG derivatives are described in U.S. Pat. Nos. 5,643,575, 5,919,455, 6,113,906 and 6,566,506, the disclosures of each of which are incorporated herein by reference.

The multi-armed polymers prior to the conjugation to the compounds described herein include multi-arm PEG-OH or “star-PEG” products such as those described in NOF Corp. Drug Delivery System catalog, Ver. 8, April 2006, the disclosure of which is incorporated herein by reference. Specifically, such PEG can be of the formula:

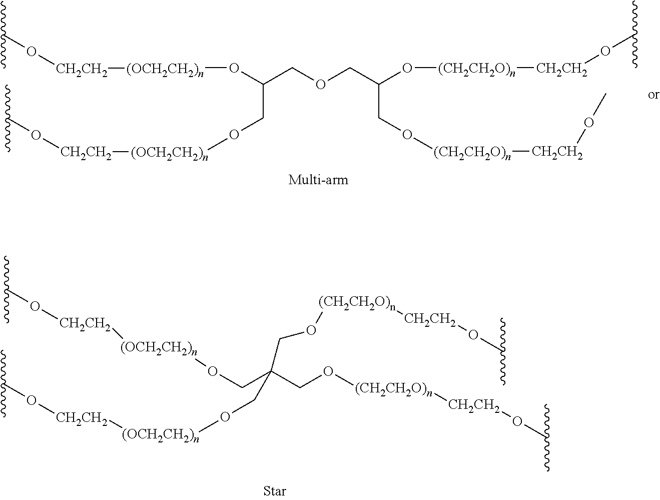

wherein:

(n) is an integer from about 4 to about 455; and up to 3 terminal portions of the residue is/are capped with a methyl or other lower alkyl.

In one embodiment, the degree of polymerization for the polymer (n) is from about 28 to about 341 to provide polymers having a total number average molecular weight of from about 5,000 Da to about 60,000 Da, and preferably from about 114 to about 239 to provide polymers having a total number average molecular weight of from about 20,000 Da to about 42,000 Da. (n) represents the number of repeating units in the polymer chain and is dependent on the molecular weight of the polymer. In one particular embodiment, (n) is about 227 to provide the polymeric portion having a total number average molecular weight of about 40,000 Da.

In certain embodiments, all four of the PEG arms can be converted to suitable activating groups, for facilitating attachment to other molecules (e.g., bifunctional linkers). Such compounds prior to conversion include:

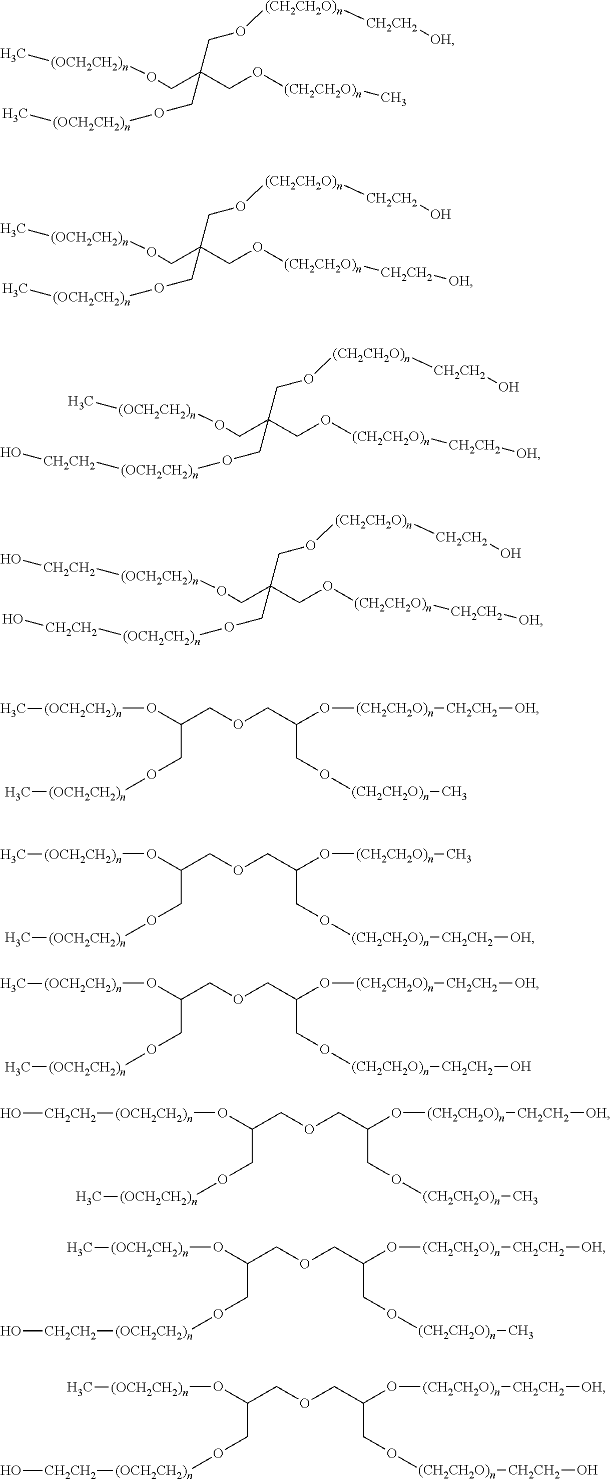

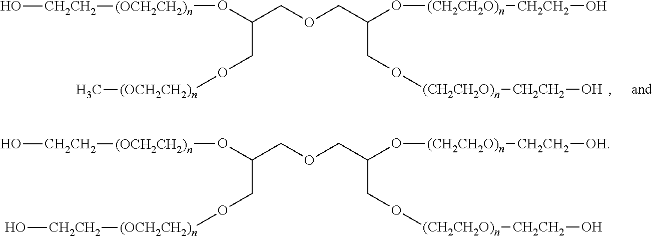

PEG may be conjugated to the compounds described herein directly or via a linker moiety. The polymers for conjugation to a compound of Formula (I) are converted into a suitably activated polymer, using the activation techniques described in U.S. Pat. Nos. 5,122,614 and 5,808,096 and other techniques known in the art without undue experimentation.

Examples of activated PEGs useful for the preparation of a compound of Formula (I) include, for example, methoxypolyethylene glycol-succinate, methoxypolyethylene glycol-succinimidyl succinate (mPEG-NHS), methoxypolyethyleneglycol-acetic acid (mPEG-CH₂COOH), methoxypolyethylene glycol-amine (mPEG-NH₂), and methoxypolyethylene glycol-tresylate (mPEG-TRES).

In certain aspects, polymers having terminal carboxylic acid groups can be employed in the compounds described herein. Methods of preparing polymers having terminal carboxylic acids in high purity are described in U.S. patent application Ser. No. 11/328,662, the contents of which are incorporated herein by reference.

In alternative aspects, polymers having terminal amine groups can be employed to make the compounds described herein. The methods of preparing polymers containing terminal amines in high purity are described in U.S. Pat. Nos. 7,569,657 and 7,868,131, the contents of each of which are incorporated herein by reference.

In yet a further aspect of the invention, the polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In yet a further embodiment and as an alternative to PAO-based polymers such as PEG, one or more effectively non-antigenic materials such as dextran, polyvinyl alcohols, carbohydrate-based polymers, hydroxypropylmethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “substantially or effectively non-antigenic” means polymeric materials understood in the art as being nontoxic and not eliciting an appreciable immunogenic response in mammals.

### E. Targeting Groups

In another aspect, the compounds described herein include a targeting ligand for a specific cell of tissue type. Any known techniques in the art can be used for conjugating a targeting group to the compounds of Formula (I) without undue experimentation.

For example, targeting agents can be attached to the compounds described herein to guide the conjugates to the target area in vivo. The targeted delivery of the compounds described herein enhances the cellular uptake of the compounds described herein, thereby improving the therapeutic efficacies. In certain aspects, some cell penetrating peptides can be replaced with a variety of targeting peptides for targeted delivery to the tumor site.

In one embodiment, the targeting moiety, such as a single chain antibody (SCA) or single-chain antigen-binding antibody, monoclonal antibody, cell adhesion peptides such as RGD peptides and Selectin, cell penetrating peptides (CPPs) such as TAT, Penetratin and (Arg)₉, receptor ligands, targeting carbohydrate molecules or lectins allows the compounds described herein to be specifically directed to targeted regions. See J Pharm Sci. 2006 September; 95(9):1856-72 Cell adhesion molecules for targeted drug delivery, the contents of which are incorporated herein by reference.

Suitable targeting moieties include single-chain antibodies (SCA's) or single-chain variable fragments of antibodies (sFv). The SCA contains domains of antibodies which can bind or recognize specific molecules of targeting tumor cells.

The terms “single chain antibody” (SCA), “single-chain antigen-binding molecule or antibody” or “single-chain Fv” (sFv) are used interchangeably. The single chain antibody has binding affinity for the antigen. Single chain antibody (SCA) or single-chain Fvs can and have been constructed in several ways. A description of the theory and production of single-chain antigen-binding proteins is found in commonly assigned U.S. patent application Ser. No. 10/915,069 and U.S. Pat. No. 6,824,782, the contents of each of which are incorporated by reference herein.

Typically, SCA or Fv domains can be selected among monoclonal antibodies known by their abbreviations in the literature as 26-10, MOPC 315, 741F8, 520C9, McPC 603, D1.3, murine phOx, human phOx, RFL3.8 sTCR, 1A6, Se155-4, 18-2-3, 4-4-20, 7A4-1, B6.2, CC49, 3C2, 2c, MA-15C5/K₁₂G_{O}, Ox, etc. (see, Huston, J. S. et al., Proc. Natl. Acad. Sci. USA 85:5879-5883 (1988); Huston, J. S. et al., SIM News 38(4) (Supp):11 (1988); McCartney, J. et al., ICSU Short Reports 10:114 (1990); McCartney, J. E. et al., unpublished results (1990); Nedelman, M. A. et al., J. Nuclear Med. 32 (Supp.):1005 (1991); Huston, J. S. et al., In: Molecular Design and Modeling: Concepts and Applications, Part B, edited by J. J. Langone, Methods in Enzymology 203:46-88 (1991); Huston, J. S. et al., In: Advances in the Applications of Monoclonal Antibodies in Clinical Oncology, Epenetos, A. A. (Ed.), London, Chapman & Hall (1993); Bird, R. E. et al., Science 242:423-426 (1988); Bedzyk, W. D. et al., J. Biol. Chem. 265:18615-18620 (1990); Colcher, D. et al., J. Nat. Cancer Inst. 82:1191-1197 (1990); Gibbs, R. A. et al., Proc. Natl. Acad. Sci. USA 88:4001-4004 (1991); Milenic, D. E. et al., Cancer Research 51:6363-6371 (1991); Pantoliano, M. W. et al., Biochemistry 30:10117-10125 (1991); Chaudhary, V. K. et al., Nature 339:394-397 (1989); Chaudhary, V. K. et al., Proc. Natl. Acad. Sci. USA 87:1066-1070 (1990); Batra, J. K. et al., Biochem. Biophys. Res. Comm. 171:1-6 (1990); Batra, J. K. et al., J. Biol. Chem. 265:15198-15202 (1990); Chaudhary, V. K. et al., Proc. Natl. Acad. Sci. USA 87:9491-9494 (1990); Batra, J. K. et al., Mol. Cell. Biol. 11:2200-2205 (1991); Brinkmann, U. et al., Proc. Natl. Acad. Sci. USA 88:8616-8620 (1991); Seetharam, S. et al., J. Biol. Chem. 266:17376-17381 (1991); Brinkmann, U. et al., Proc. Natl. Acad. Sci. USA 89:3075-3079 (1992); Glockshuber, R. et al., Biochemistry 29:1362-1367 (1990); Skerra, A. et al., Bio/Technol. 9:273-278 (1991); Pack, P. et al., Biochemistry 31:1579-1534 (1992); Clackson, T. et al., Nature 352:624-628 (1991); Marks, J. D. et al., J. Mol. Biol. 222:581-597 (1991); Iverson, B. L. et al., Science 249:659-662 (1990); Roberts, V. A. et al., Proc. Natl. Acad. Sci. USA 87:6654-6658 (1990); Condra, J. H. et al., J. Biol. Chem. 265:2292-2295 (1990); Laroche, Y. et al., J. Biol. Chem. 266:16343-16349 (1991); Holvoet, P. et al., J. Biol. Chem. 266:19717-19724 (1991); Anand, N. N. et al., J. Biol. Chem. 266:21874-21879 (1991); Fuchs, P. et al., Biol Technol. 9:1369-1372 (1991); Breitling, F. et al., Gene 104:104-153 (1991); Seehaus, T. et al., Gene 114:235-237 (1992); Takkinen, K. et al., Protein Engng. 4:837-841 (1991); Dreher, M. L. et al., J. Immunol. Methods 139:197-205 (1991); Mottez, E. et al., Eur. J. Immunol. 21:467-471 (1991); Traunecker, A. et al., Proc. Natl. Acad. Sci. USA 88:8646-8650 (1991); Traunecker, A. et al., EMBO J. 10:3655-3659 (1991); Hoo, W. F. S. et al., Proc. Natl. Acad. Sci. USA 89:4759-4763 (1993)). Each of the forgoing publications is incorporated herein by reference.

A non-limiting list of targeting groups includes vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides, RGD peptides, folate, anisamide, etc. Other optional targeting agents appreciated by artisans in the art can be also employed in the compounds described herein.

In one preferred embodiment, the targeting agents useful for the compounds described herein include single chain antibody (SCA), RGD peptides, selectin, TAT, penetratin, (Arg)₉, folic acid, anisamide, etc., and some of the preferred structures of these agents are:

C-TAT:
(SEQ ID NO: 1)
CYGRKKRRQRRR;
C-(Arg)₉:
(SEQ ID NO: 2)
CRRRRRRRRR;

RGD can be linear or cyclic:

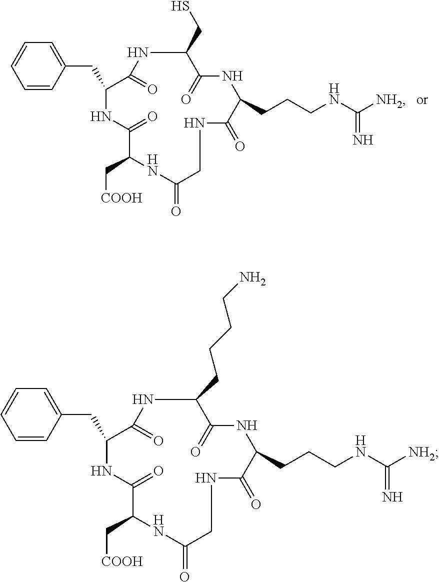

Folic acid is a residue of

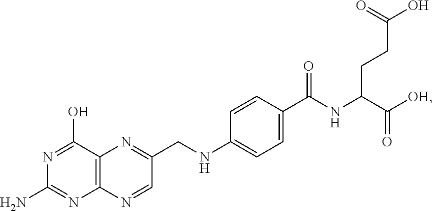

and

Anisamide is p-MeO-Ph-C(═O)OH.

Arg₉ can include a cysteine for conjugating such as CRRRRRRRRR and TAT can add an additional cysteine at the end of the peptide such as CYGRKKRRQRRRC.

For purpose of the current invention, the abbreviations used in the specification and figures represent the following structures:
(i) Linear RGD (SEQ ID NO: 3)=RGDC;
(ii) Cyclic RGD (SEQ ID NO: 4 and SEQ ID NO: 5)=c-RGDFC or c-RGDFK; and
(iii) RGD-TAT (SEQ ID NO: 6)=CYGRKKRRQRRRGGGRGDS-NH₂; and

Alternatively, the targeting group include sugars and carbohydrates such as galactose, galactosamine, and N-acetyl galactosamine; hormones such as estrogen, testosterone, progesterone, glucocortisone, adrenaline, insulin, glucagon, cortisol, vitamin D, thyroid hormone, retinoic acid, and growth hormones; growth factors such as VEGF, EGF, NGF, and PDGF; neurotransmitters such as GABA, Glutamate, acetylcholine; NOGO; inostitol triphosphate; epinephrine; norepinephrine; Nitric Oxide, peptides, vitamins such as folate and pyridoxine, drugs, antibodies and any other molecule that can interact with an cell surface receptor in vivo or in vitro.

### F. Leaving Groups and Functional Groups

In some aspects, suitable leaving groups include, without limitations, halogen (Br, Cl), activated carbonate, carbonyl imidazole, cyclic imide thione, chloroformate, isocyanate, N-hydroxysuccinimidyl, chloroformate, para-nitrophenoxy (PNP), N-hydroxyphtalimide, N-hydroxybenzotriazolyl (N-HOBT), tosylate, mesylate, tresylate, nosylate, C₁-C₆ alkyloxy, C₁-C₆ alkanoyloxy, arylcarbonyloxy, ortho-nitrophenoxy, N-hydroxybenzotriazolyl, imidazole, pentafluorophenoxy, 1,3,5-trichlorophenoxy, and 1,3,5-trifluorophenoxy or other suitable leaving groups, as will be apparent to those of ordinary skill. In one embodiment, the T₁ group can be carbonyl imidazole, chloroformate, isocyanate, or PNP.

For purposes of the present invention, leaving/activating groups are to be understood as those groups which are capable of reacting with a nucleophile found on the desired target, i.e. a bifunctional spacer, a targeting moiety, a polymer, a diagnostic agent, an intermediate, etc. The targets thus contain a group for displacement, such as OH, NH₂ or SH groups.

In some embodiments, functional groups include maleimidyl, vinyl, residues of sulfone, amino, carboxy, mercapto, hydrazide, carbazate and the like which can be further conjugated to a polymer.

In yet some preferred embodiments of the invention, the leaving/activating groups can be selected from among carbonyl imidazole, chloroformate, isocyanate, PNP, tosylate, N—HOBT, and N-hydroxysuccinimidyl.

### G. Diagnostic Agents

A further aspect of the invention provides the compounds optionally prepared with a diagnostic tag linked to the compounds described herein, wherein the tag is selected for diagnostic or imaging purposes.

The compounds described herein can be labeled or tagged. Suitable labels or tags (the terms are used interchangeably herein) include, e.g., biotinylated compounds, fluorescent compounds, and radiolabelled compounds. A suitable tag is prepared by linking any suitable moiety, e.g., an amino acid residue, to any art-standard emitting isotope, radio-opaque label, magnetic resonance label, or other non-radioactive isotopic labels suitable for magnetic resonance imaging, fluorescence-type labels, labels exhibiting visible colors and/or capable of fluorescing under ultraviolet, infrared or electrochemical stimulation, to allow for imaging tumor tissue during surgical procedures, and so forth. The diagnostic tag is incorporated into and/or linked to a therapeutic moiety (biologically active agents), allowing for monitoring of the distribution of a therapeutic biologically active material within an animal or human patient.

The inventive tagged conjugates are readily prepared, by art-known methods, with any suitable label, including, e.g., radioisotope labels. Simply by way of example, these include ¹³¹Iodine, ¹²⁵Iodine, ^{99m}Technetium and/or ¹¹¹Indium to produce radioimmunoscintigraphic agents for selective uptake into tumor cells, in vivo. For instance, there are a number of art-known methods of linking peptide to Tc-99m, including, simply by way of example, those shown by U.S. Pat. Nos. 5,328,679; 5,888,474; 5,997,844; and 5,997,845, incorporated herein by reference.

### H. Intramolecular Cyclization-Assisted Urea Linker Elimination and Native Drug Regeneration

According to the present invention, the urea-containing linker attached to biologically active agents will undergo an intramolecular cyclization to eliminate the urea-containing linker in vivo to produce parent compounds by inductive effects such as anchimeric assistance. For example, the C(═Y₁)—NR₁—[CRₐ₁Rₐ₂]ₐ—[CR_{b1}R_{b2}]_{b}-[CR_{c1}R_{c2}]_{c}—[CR_{d1}R_{d2}]_{d}-NT₁T₂ moieties of the present invention form a four to seven-membered heterocyclic transition structure (preferably, a five-membered heterocyclic transition structure) to regenerate the parent drugs, i.e., aromatic amine-containing biologically active moieties. Illustrative examples of representative reactions are shown below:

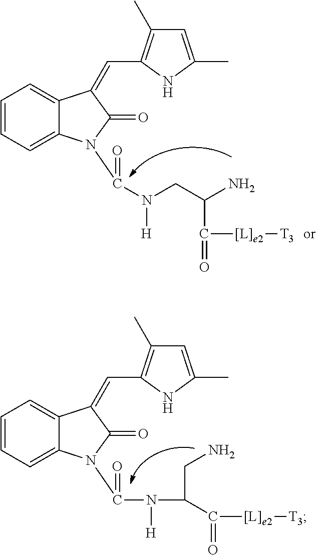

with Y₁ being 0; R₁ being H; NT₁T₂ being NH₂; and D being an amine-containing target moiety (i.e. SU5416).

The compounds include —NH—, which initiates the self-cyclization to regenerate parent drugs.

The compounds of the present invention can be designed so that the t_{1/2} of hydrolysis is <t_{1/2} elimination in vivo. The hydrolysis rates can be modified to allow sufficient amounts of the bioactive parent compounds to be released prior to elimination. In this aspect, the compounds described herein can include a polymer to extend the circulation of the compounds, prior to the hydrolysis. In one embodiment, the compounds include:

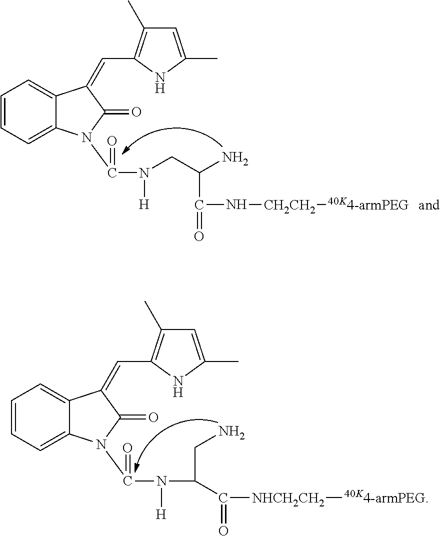

In a further aspect, the elimination of the urea linker can be initiated by an additional cleavage. The initial cleavage can be based on another cleavage reaction by an enzyme (i.e., esterase) or pH. The compounds are stable, until the first cleavage takes places in vivo in mammals being treated. The initial cleavage provides —NH— which can proceed with the self-cyclization to regenerate biologically active parent compounds. Once the first cleavage occurs, the resulting compound undergoes the urea linker elimination and produces the target drug. Illustrative examples of such cleavage reactions are shown below:

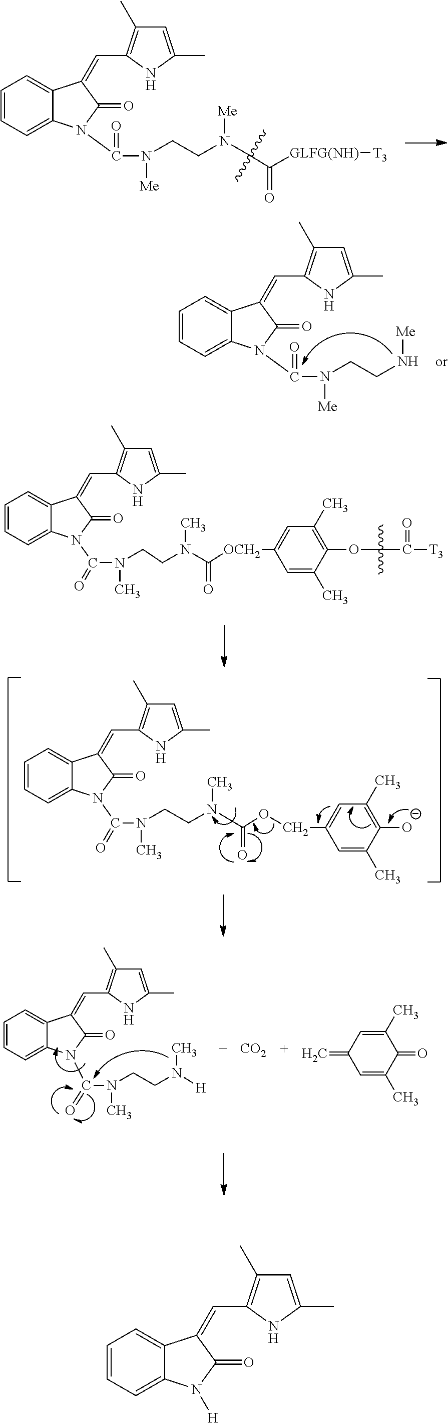

One embodiment with an alternative prodrug system includes, without limitation:

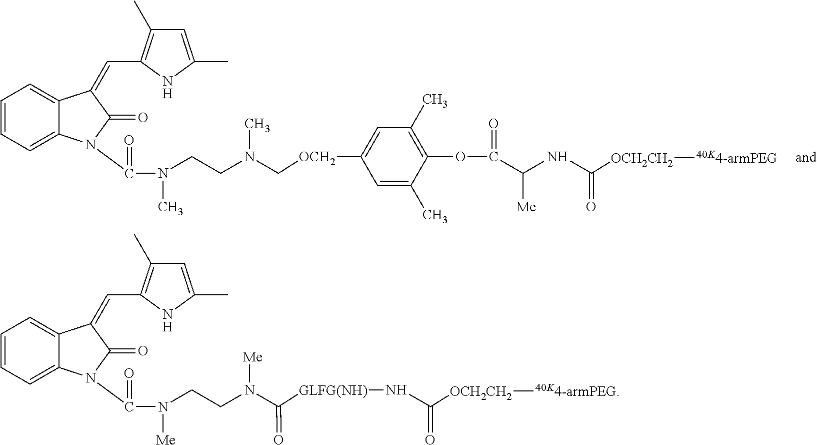

### I. Synthesis of Compounds of Formula (I)

Generally, compounds described herein are prepared by coupling an aromatic amine-containing compound (e.g., SU5416) with a bifunctional linker to form a urea linker, followed by reacting one or more equivalents of the resulting intermediate with an activated polymer under conditions which are sufficient to form a compound of Formula (I). Synthesis of representative compounds is set forth in the Examples. However, the compounds described herein can be prepared in several fashions.

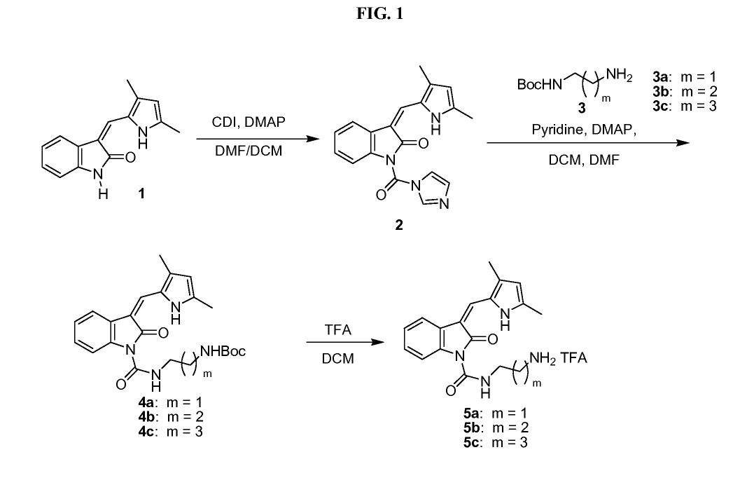

In one embodiment, an aromatic amine-containing compound (e.g., SU5416) is activated with chloroformate or carbonyldiimidazole under basic conditions. The activated compound is reacted with an amine moiety of a mono-protected bifunctional linker to form a urea linkage. The resulting intermediate compound is deblocked. Further, the deblocked compound is reacted with an activated polymer such as SC-PEG or PEG-COOH to form a polymeric compound containing a releasable urea linker system. For example, as shown in FIG. 1, the amine of compound 1 is activated by reacting with an acylating agent such as carbonyldiimidazole (CDI) under basic conditions. The activated compound (compound 2) is then reacted with a mono amine-protected bifunctional linker (compound 3). After deprotection, the protected bifunctional linker-SU5416 intermediate is coupled with an activated polymer under basic conditions to form a polymeric conjugate containing a releasable urea linker system.

A non-limiting list of acylating agents includes phosgene, triphosgene, disuccinimidyl carbonate, carbonyl diimidazole, para-nitrophenyl chloroformate, N-chlorocarbonyloxyphthalimide and diphthalimido carbonate.

Alternatively, an aromatic amine-containing compound (e.g., indolinone-containing tyrosine kinase inhibitors) is first treated with a strong base such as KOH or potassium t-butoxide, and the nitrogen of the compound is deprotonated. The deprotonated compound is reacted with an activated mono-protected bifunctional acyl linker. The resulting intermediate is deprotected with an acid, and reacted with an activated polymer to form a polymeric conjugate containing a releasable urea linker system under coupling conditions.

More specifically, methods described herein can include:

1) treating one equivalent of an aromatic amine-containing compound (e.g., SU5416) with a strong base such as KOH or potassium tert-butoxide to provide a nitrogen anion, followed by reacting with an activated bifunctional linker to form an acyl derivative of the aromatic amine-containing compound, or

reacting one equivalent of an aromatic amine-containing compound with one or more equivalents of a bifunctinal linker containing an activated amine group, such as isocyanate, in the presence of base to form an acyl derivative of the aromatic amine-containing compound;

wherein, the bifunctional linkers contain a secondary functional group in a protected form;

2) deprotecting the secondary functional group in the bifunctional linker with a strong acid or base; and

3) reacting the resulting aromatic amine-containing compound-bifunctional linker intermediate with an activated polymer, such as PEG-succinimidyl carbonate in an inert solvent such as DCM (or DMF, chloroform, toluene or mixtures thereof) in the presence of a base, or PEG-carboxylic acid in the presence of a coupling reagent such as 1,(3-dimethyl aminopropyl) 3-ethyl carbodiimide (EDC), PPAC (or 1,3-diisopropylcarbodiimide (DIPC), any suitable dialkyl carbodiimide, Mukaiyama reagents, (e.g. 2-halo-1-alkyl-pyridinium halides) or propane phosphonic acid cyclic anhydride (PPACA), etc.), with a suitable base such as DMAP at a temperature from 0° C. up to 22° C.

The activated polymer, i.e., a polymer containing one up to four terminal carboxyl acid groups can be prepared by converting NOF Sunbright-type, Star-shaped, or other branched polymers having terminal OH groups into corresponding carboxyl acid derivatives using techniques described in U.S. Pat. No. 5,605,976, the contents of which are incorporated herein by reference.

Compounds prepared according to the present invention include:

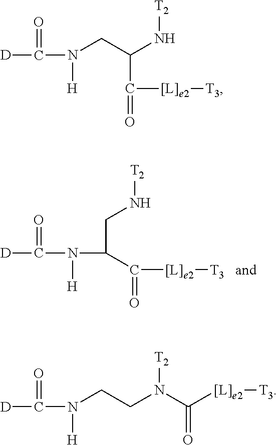

In one embodiment, the biologically active agent is an indolinone-based tyrosine kinase inhibitor such as SU5416 (Semaxanib). Examples of the compounds include:

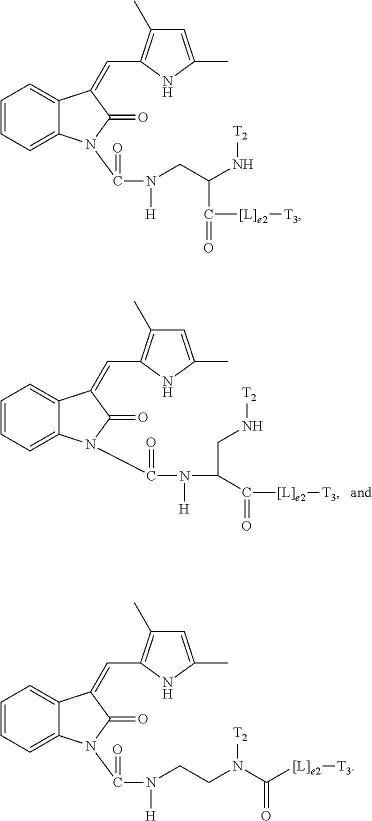

For example, the compounds of Formula (I) prepared by the methods described herein can be among:

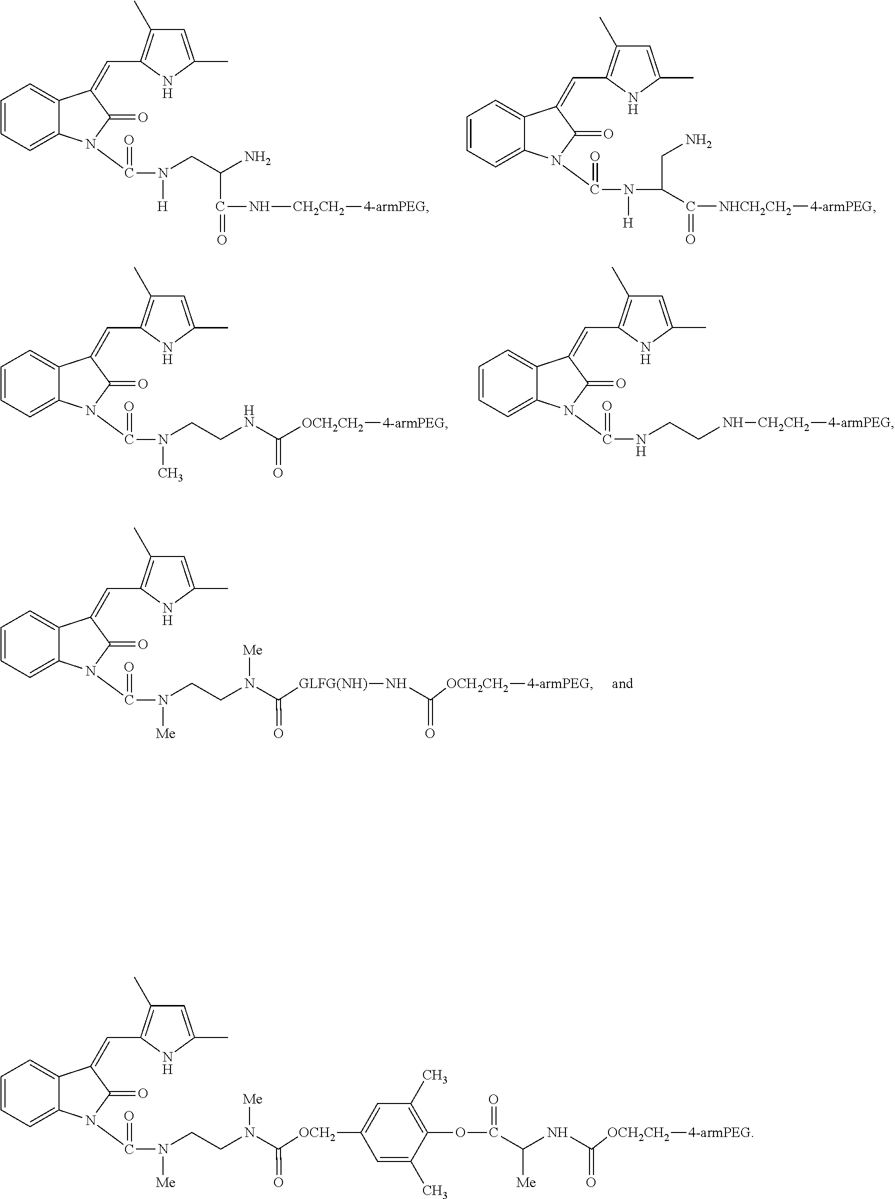

For ease of the description and not limitation, only one arm of the four-arm PEG is shown. One arm, up to four arms of the four-arm PEG can be conjugated with biologically active agents such as SU5416.

A non-limiting list of compounds prepared by the methods described herein includes:

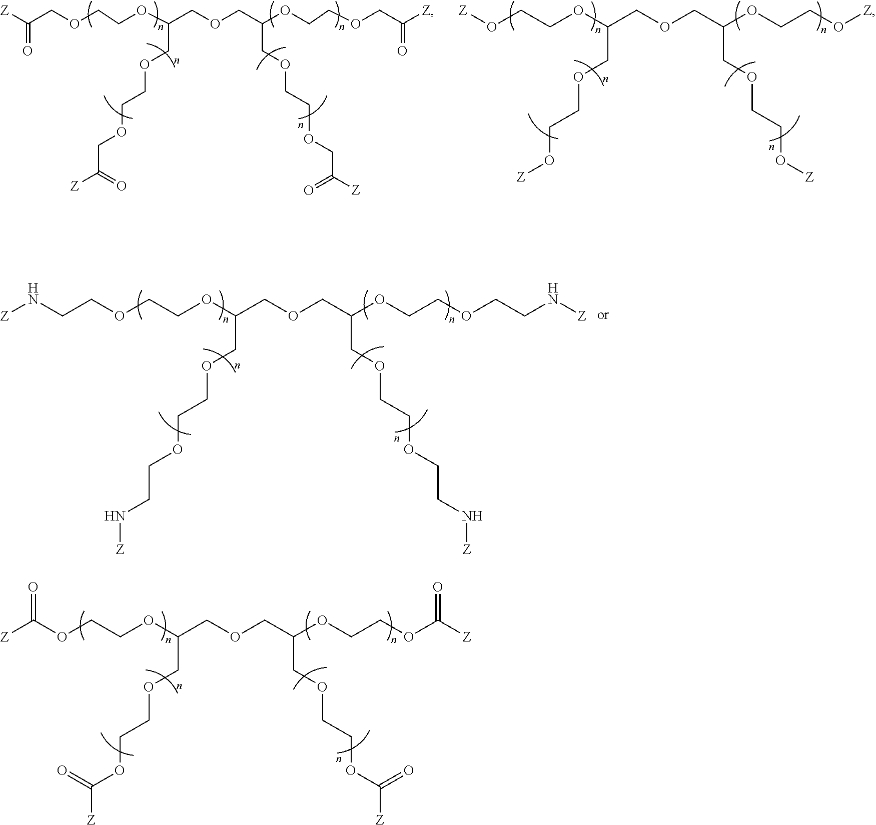

wherein Z is selected from among:

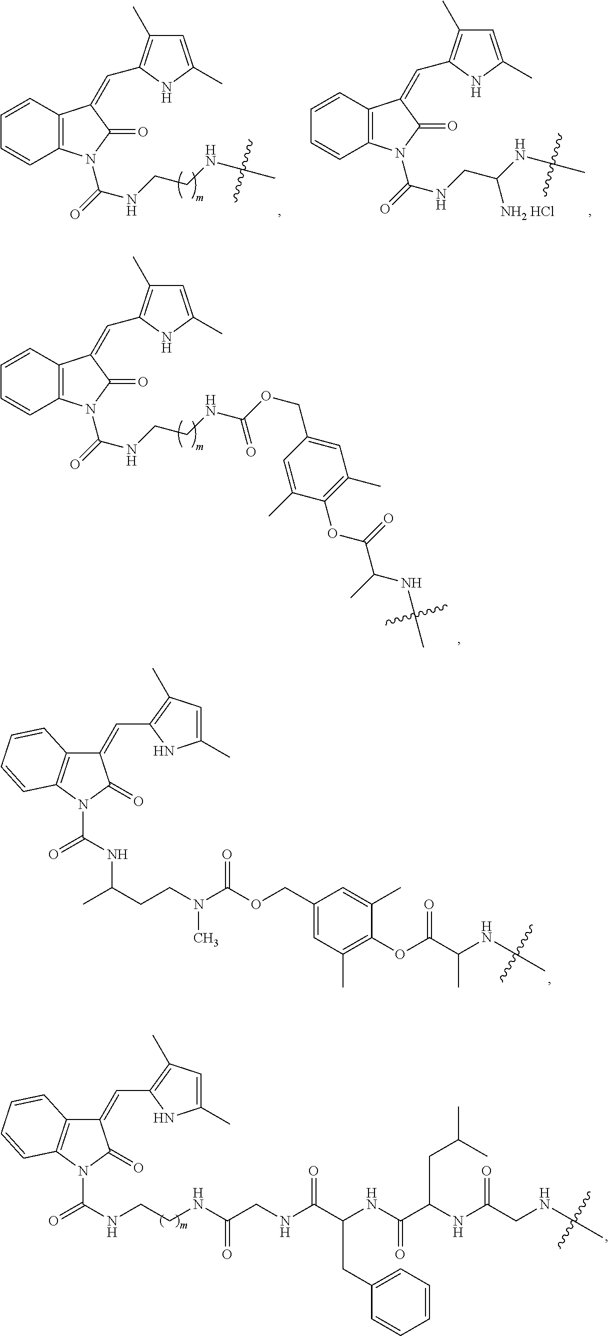

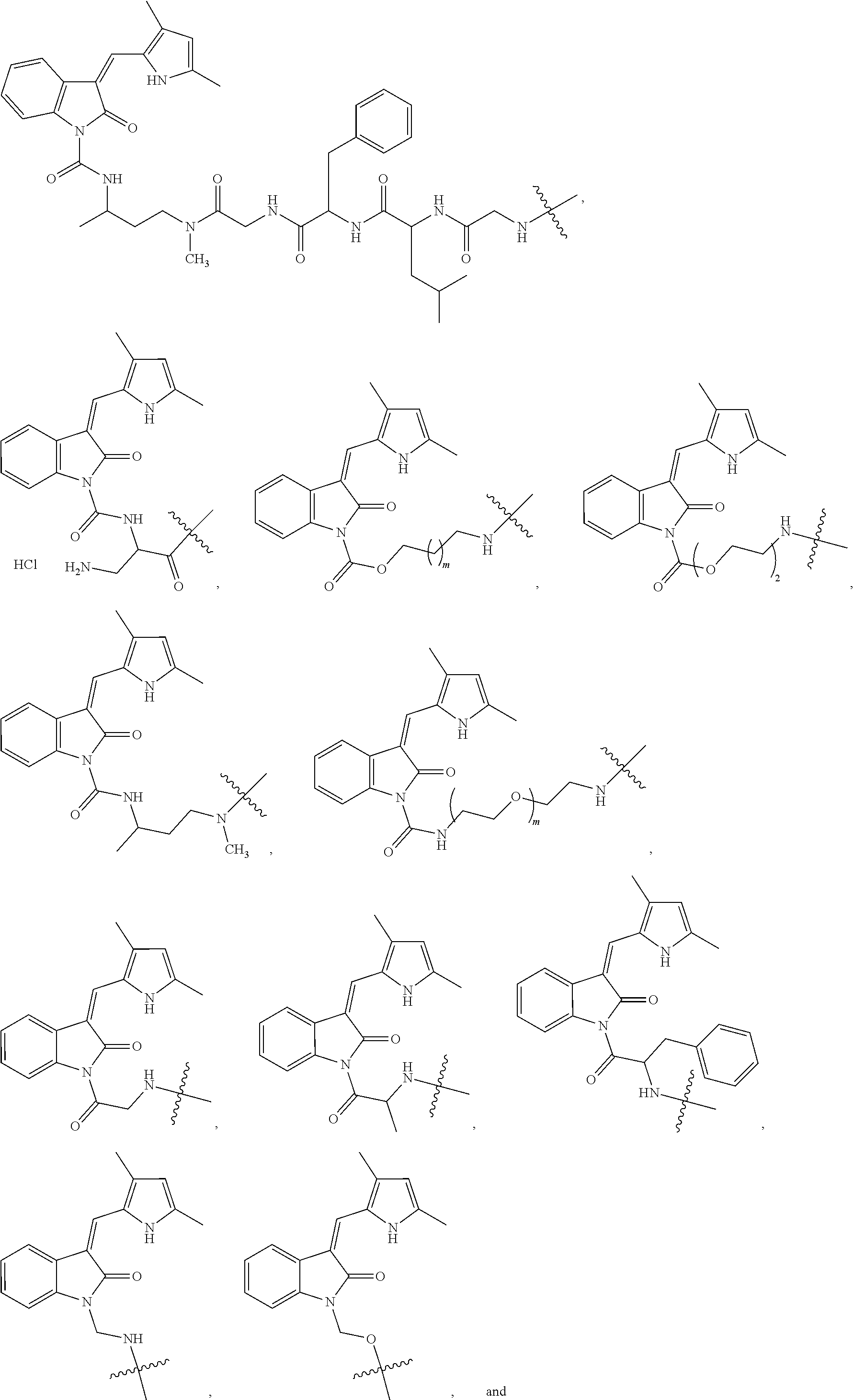

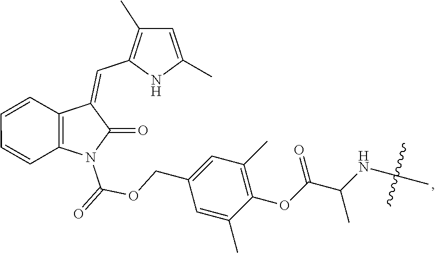

wherein (m) is 1, 2, or 3.

Preferably, four arms of the polymers are conjugated to indolinone or its derivatives through a linker. HPLC analysis of compounds made in accordance with this aspect of the inventions shows that on average about four indolinone or its derivative molecules are conjugated to one PEG molecule (about 2% by weight).

One preferred embodiment includes compounds having the structure:

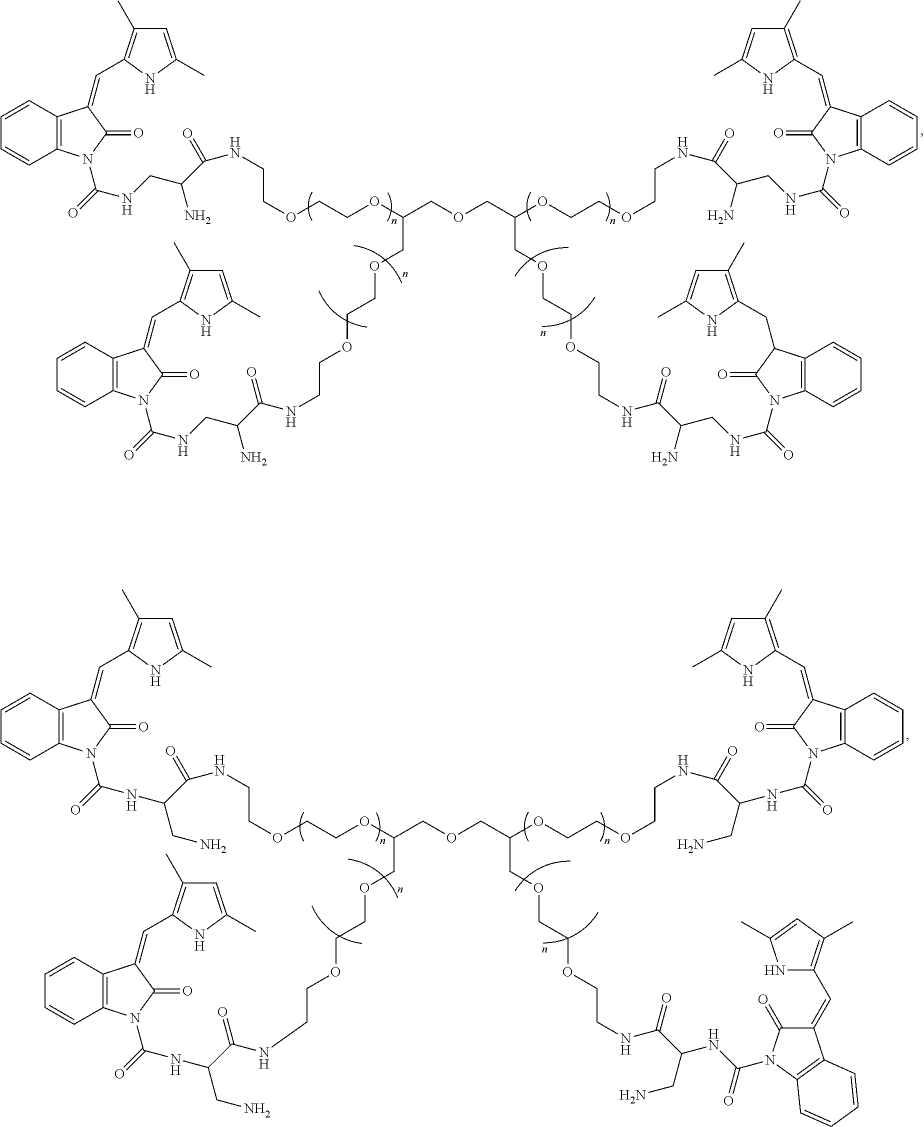

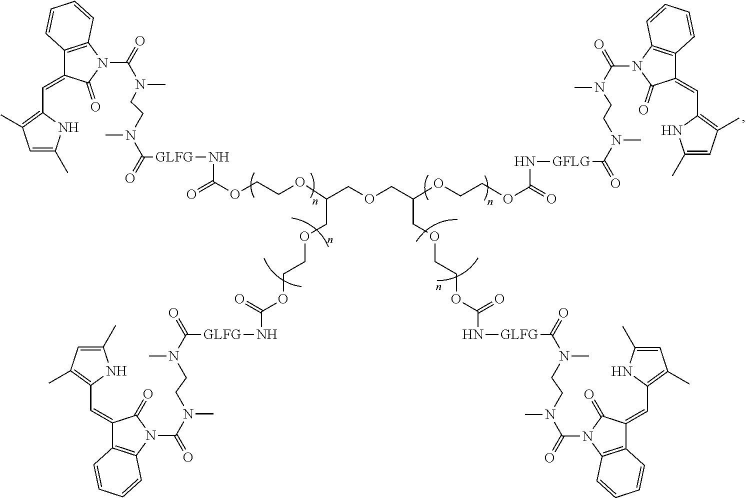

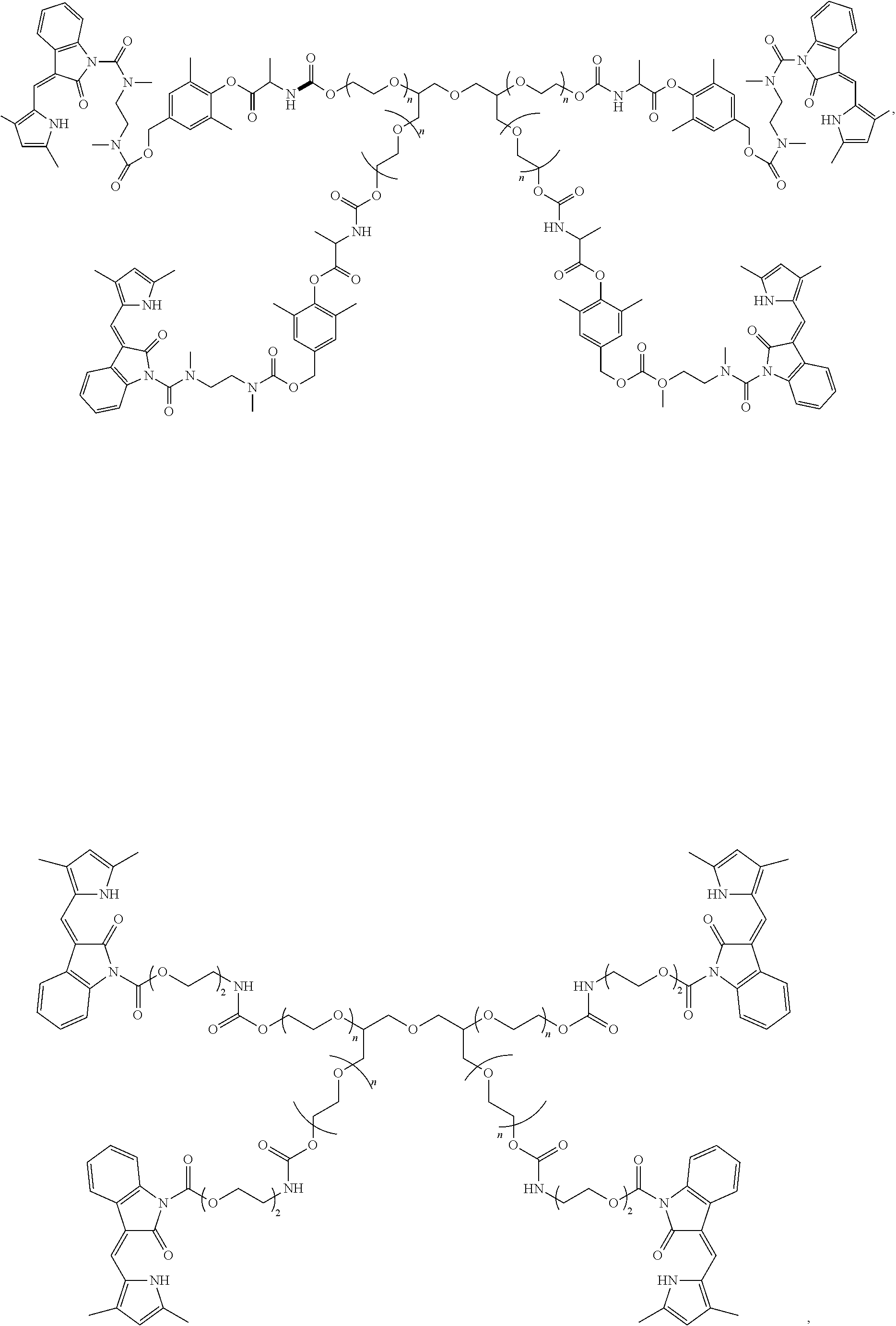

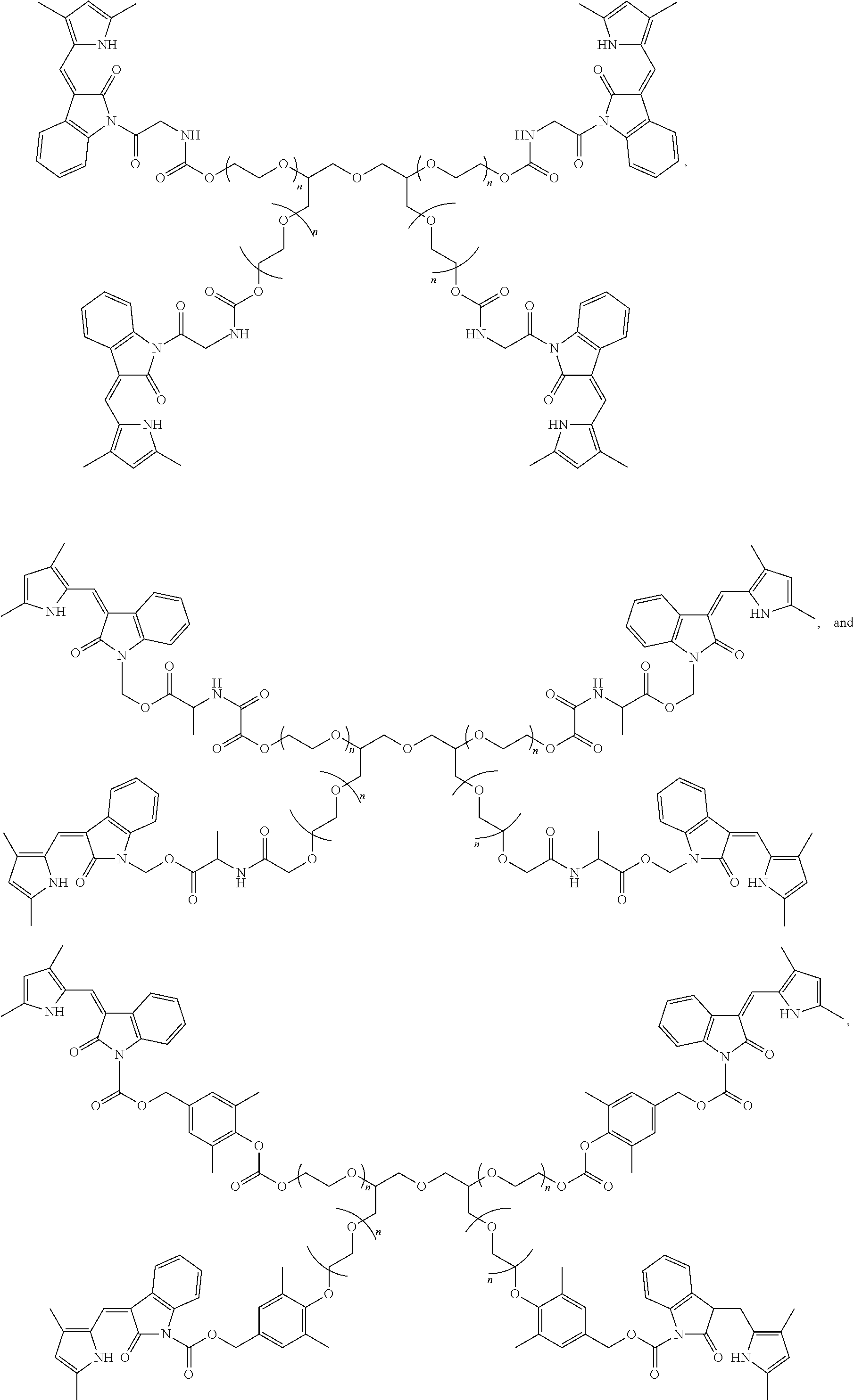

wherein (n) is an integer from about 10 to about 2,300 and the polymer portion has a total molecular weight of about 40,000 daltons. The N-terminal of the peptide, e.g. -GLFG-(SEQ ID NO: 7), is specified as —NH— and the C-terminal as C(═O). Therefore, —C(═O)-GLFG-NH— or —HN-GFLG-C(═O)— is residue of peptide GLFG, -Gly-Leu-Phe-Gly- from C-terminal to N-terminal.

### J. Compositions/Formulations

Pharmaceutical compositions containing the compounds of the present invention may be manufactured by processes well known in the art, e.g., using a variety of well-known mixing, dissolving, granulating, levigating, emulsifying, encapsulating, entrapping or lyophilizing processes. The compositions may be formulated in conjunction with one or more physiologically acceptable carriers comprising excipients and auxiliaries which facilitate processing of the active compounds into preparations which can be used pharmaceutically. Proper formulation is dependent upon the route of administration chosen. Parenteral routes are preferred in many aspects of the invention. For injection, including, without limitation, intravenous, intramusclular and subcutaneous injection, the compounds of the invention may be formulated in aqueous solutions, preferably in physiologically compatible buffers such as physiological saline buffer or polar solvents including, without limitation, a pyrrolidone or dimethylsulfoxide.

The compounds described herein may also be formulated for parenteral administration, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt (preferred) of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use.

For oral administration, the compounds can be formulated by combining the compounds described herein with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl-methylcellulose, sodium carboxy-methylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Other delivery systems such as liposomes and emulsions can also be used.

Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the particular compound, additional stabilization strategies may be employed.

### K. Use of Compounds of Formula (I)

In one aspect of the present invention, the compounds of the present invention can be useful in the delivery of aromatic amine-containing biologically active agents into the body in mammals. The methods include administering the compounds described herein to a mammal in need thereof. One embodiment according to the present invention includes

(a) forming a polymeric conjugate of an aromatic amine-containing biologically active agent; and

(b) administering the conjugate to a mammal in need thereof,

wherein the conjugate is represented by Formula (I).

In one particular embodiment, the present invention provides methods of delivering an indolinone derivative to a mammal. The methods include

(a) forming a polymeric conjugate of an indolinone-based tyrosine kinase inhibitor; and

(b) administering the conjugate to a mammal in need thereof,

wherein the conjugate is represented by Formula (I).

Another aspect of the present invention provides methods of treatment for various medical conditions in mammals.

In one embodiment, there are provided methods of treating a patient having a malignant tumor or cancer, comprising administering an effective amount of a pharmaceutical composition containing the compounds described herein to a patient in need thereof, wherein D is a biologically active moiety. The cancer being treated can be one or more of the following: solid tumors, lymphomas, small cell lung cancer, acute myeloid leukemia (AML), acute lymphocytic leukemia (ALL), pancreatic cancer, glioblastoma, ovarian cancer, gastric cancers, colorectal cancer, prostate cancer, cervical cancer, brain tumors, KB cancer, lung cancer, colon cancer, epidermal cancer, etc. The compounds of the present invention are useful for treating neoplastic disease, reducing tumor burden, preventing metastasis of neoplasms and preventing recurrences of tumor/neoplastic growths in mammals.

In this aspect, “treatment” or “cure” shall be understood to mean inhibition, reduction, amelioration and prevention of tumor growth, tumor burden and metastasis, remission of tumor, or prevention of recurrences of tumor and/or neoplastic growths in patients after completion of treatment.

Treatment is deemed to occur when a patient achieves positive clinical results. For example, successful treatment shall be deemed to occur when at least 20% or preferably 30%, more preferably 40% or higher (i.e., 50%) decrease in tumor growth including other clinical markers contemplated by the artisan in the field is realized when compared to that observed in the absence of the treatment described herein.

In certain aspects, clinical response criteria defined according to RECIST guidelines can be useful. Complete response (CR) is defined as complete disappearance of measurable and evaluable clinical evidence of cancer. Partial response (PR) is defined as at least a 50% reduction in the size of all measurable tumor areas. Progressive disease (PD) is defined as an increase of >25% (compared to baseline or best response) in the size of all measurable tumor areas. Stable disease (SD) is defined as neither sufficient shrinkage to qualify for PR nor sufficient increase to qualify for PD. Treatment is deemed to occur, when CR, PR and/or SD are achieved.

In another and further embodiment, the present invention provides methods of treating tyrosine kinase-dependent diseases or conditions. The methods include administering a compound of the present invention to a patient in need thereof, wherein D is a tyrosine kinase inhibitor such as indolinone-based tyrosine kinase inhibitors. In one preferred embodiment, D is SU5416.

The term “tyrosine kinase-dependent diseases or conditions” refers to pathological conditions that depend on the abnormal activity of one or more tyrosine kinases. Abnormal tyrosine kinase activities are associated with disorders such as uncontrolled angiogenesis and/or vasculogenesis. Diseases associated with abnormal tyrosine kinase activities include the proliferation of tumor cells, the pathologic neovascularization that supports solid tumor growth, ocular neovascularization (diabetic retinopathy, age-related macular degeneration, and the like) and inflammation (psoriasis, rheumatoid arthritis, and the like). Tyrosine kinase related disorders are commonly associated with an increase in the catalytic activity of the tyrosine kinases, where the tyrosine kinases can be receptor protein tyrosine kinases, and non-receptor or cellular tyrosine kinases.

Yet another embodiment according to the present invention provides methods of modulating/inhibiting angiogenesis or angiogenic activity in a mammal. The angiogenesis is a tumoral angiogenesis or tumor-dependent angiogenesis.

In yet a further embodiment, the methods described herein can be useful in the treatment of patients with diseases associated with abnormally high levels of VEGF expression, as compared to normal subjects. Levels of VEGF expression can be measured by techniques known in the art, including the measurement of VEGF mRNA expression.

In many aspects of the present invention, the methods employ use of compounds of Formula (I) or pharmaceutical salt thereof to a mammal in need thereof, wherein D is an indolinone-based tyrosine kinase inhibitor.

In one embodiment, the methods described herein employ SU5416. SU5416 inhibits Flt-1 tyrosine kinase activity and KDR/Flk-1 tyrosine kinase activity. SU5416 is a potent inhibitor of tumor angiogenesis. SU5416 inhibits Flt-1 tyrosine kinase activity and KDR/Flk-1 tyrosine kinase activity.

A therapeutically effective amount means an amount of compound effective to prevent, alleviate or ameliorate symptoms of disease or prolong the survival of the subject being treated with the compounds described herein. For compounds used in the methods described herein, the therapeutically effective amount can be estimated initially from in vitro assays. Then, the dosage can be formulated for use in animal models so as to achieve a circulating concentration range that includes the effective dosage. Such information can be used to more accurately determine dosages useful in patients.

The amount of the composition, e.g., used as a prodrug, that is administered will depend upon the parent molecule included therein. Generally, the amount of prodrug used in the treatment methods is that amount which effectively achieves the desired therapeutic result in mammals. Naturally, the dosages of the various prodrug compounds can vary somewhat depending upon the parent compound, rate of in vivo hydrolysis, molecular weight of the polymer, etc. In addition, the dosage, of course, can vary depending upon the dosage form and route of administration.

In general, indolinone-based tyrosine kinase inhibitors are administered to mammals in amounts ranging from about 10 to about 55 mg/kg/dose. For example, the indolinone-based tyrosine kinase inhibitors such as SU5416 can be given in amounts of from about 15 to about 25 mg/kg daily or about 50 mg/kg twice or three times weekly.

Alternatively, the indolinone-based tyrosine kinase inhibitors can be administered in amounts of from about 30 to about 150 mg/m³/dose (e.g., from about 50 to about 150 mg/m³, from about 70 to about 150 mg/m³, from about 100 to about 150 mg/m³). In one embodiment, SU5416 is administered intravenously to a patient at a dose of about 145 mg/m³ twice weekly.

The treatment protocol can be based on a single dose treatment protocol or divided into multiple doses which are given as part of a multi-week treatment protocol. It is also contemplated that the treatment will be given for one or more cycles until the desired clinical result is obtained.

For purposes of the present invention, the weight given above represents the weight of the regenerated biologically active parent compound present in the compounds of Formula (I) employed in the methods described herein.

The range set forth above is illustrative and those skilled in the art will determine the optimal dosing of the prodrug selected based on clinical experience and the treatment indication. Moreover, the exact formulation, route of administration and dosage can be selected by the individual physician in view of the patient's condition. The precise dose will depend on the stage and severity of the condition, and the individual characteristics of the patient being treated, as will be appreciated by one of ordinary skill in the art.

Additionally, toxicity and therapeutic efficacy of the compounds described herein can be determined by standard pharmaceutical procedures in cell cultures or experimental animals using methods well-known in the art.

Further aspects of the present invention include combining the compounds described herein with other anticancer therapies (e.g., radiotherapy or chemotherapies employing other chemotherapeutic agents) for synergistic or additive benefit. Thus, the compounds described herein can be administered prior to, during, or after other anticancer therapy. One embodiment includes concurrent administration of compounds described herein and radiotherapy in cancer treatment.

### EXAMPLES

The following examples serve to provide further appreciation of the invention but are not meant in any way to restrict the effective scope of the invention. The following examples serve to provide further appreciation of the invention but are not meant in any way to restrict the effective scope of the invention. The underlined and bold-faced numbers recited in the Examples correspond to those shown in the Figures.

### General.

All reactions were run under an atmosphere of dry nitrogen or argon. Commercial reagents were used without further purification. All PEG compounds were dried under vacuum or by azeotropic distillation (toluene) prior to use.

### Abbreviations.

DCM (dichloromethane), DIEA (N,N-diisopropylethylamine), DMAP (4-(dimethylamino)pyridine), DMF (N,N-dimethylformamide), DSC(N,N′-disuccinimidyl carbonate), EDC (1-ethyl-3-(3-dimethylaminopropyl)carbodiimide), IPA (2-propanol), TBDMS-Cl (tert-butyl dimethyl silyl chloride), TFA (trifluoroacetic acid), TEAA (tetraethylammonium acetate).

### Example 1

### General NMR Method

¹H spectra were obtained with a Varian MercuryVX-300 instrument using deuteriochloroform as solvent unless specified. ¹³C NMR spectra were obtained at 75.46 MHz on the Varian MercuryVX-300. Chemical shifts (δ) are reported in parts per million (ppm) downfield from tetramethylsilane (TMS) and coupling constants (J values) are given in hertz (Hz).

### Example 2

### HPLC Method

Analytical HPLC's were performed using a size exclusion column (PolySep-GFC-P3000, Phenomenex) under isocratic conditions with a 1:1 mixture (v/v) of methanol-water as mobile phase. Peak elution was monitored at 280 nm using a UV detector. To detect the presence of any free PEG and to confirm the presence of PEGylated conjugates, an evaporative light scattering detector (ELSD), Model 5000 ELSD (Alltech) was employed. Based on ELSD and UV analysis, all the final PEGylated products were free of native drug and were 95% pure by HPLC.

### Example 3

### Analysis of Parent Molecule Content in PEG Conjugates

Amounts of aromatic amine-containing compounds included in polymeric conjugates were studied. UV absorbance of an aromatic amine-containing compound (e.g., SU5416) in 90% MeOH in H₂O (v/v) was determined at 280 nm in five different concentrations ranging from 0.02 μmol/mL to 0.10 μmol/mL. From the standard plot of absorbance vs. concentration, the absorption coefficient (c) was calculated (O.D. at 280 nm for 1 mg/mL with 1.0 cm light path). PEGylated conjugates of aromatic-amine containing compounds were dissolved in 90% MeOH in H₂O (v/v) at an approximate concentration of 0.006 μmol/mL (based on MW of 40,000) and the UV absorbance of the compounds at 280 nm was determined. Using the value and employing the absorption coefficient (c), concentrations of aromatic amine-containing compounds in test samples were determined.

### Example 4

### Hydrolysis Rate of Compounds of Formula (I)

The rates of hydrolysis were measured by employing a C8 reversed phase column (Zorbax® SB-C8) using a gradient mobile phase consisting of (a) 0.1 M triethylammonium acetate buffer and (b) acetonitrile. A flow rate of 1 mL/min was used, and chromatograms were monitored using a UV detector at 280 nm for aromatic amine-containing compounds (e.g. SU5416). For hydrolysis in plasma, test compounds were dissolved in acetonitrile at a concentration of 20 mg/mL. The solution was divided into vials with 100 μL and the solvent removed in vacuo. To the resulting residue, 100 μL of plasma was added, then vortexed for 10 sec. The solution was incubated at 37° C. for various periods of time. A mixture of methanol-acetonitrile (1:1, v/v, 400 μL) was added to a vial at the proper interval and the mixture was vortexed for 1 min, followed by filtration through 0.45 mm filter membrane (optionally followed by a second filtration through 0.2 mm filter membrane). An aliquot of 40 μL of the filtrate was injected into the HPLC. Amounts of parent compounds and polymeric conjugates were calculated based on peak areas, and the half life of each test compound in different media was calculated using linear regression analysis from the disappearance of polymeric conjugates.

### Example 5

### Preparation of Compound 2

Compound 1 (200 mg, 0.836 mmol) was dissolved in 2 mL of DMF and 2 mL of DCM, followed by addition of CDI (271 mg, 1.67 mmol), DMAP (101 mg, 0.836 mmol) and pyridine (135 μL, 1.672 mmol). The reaction mixture was stirred overnight at room temperature, and the product was isolated. The product was used without further purification.

### Example 6

### Preparation of Compound 4

Compound 4a: A mixture of compound 2 (1 mmol), compound 3a (2 mmol), pyridine (2 mmol), and DMAP (2 mmol) in anhydrous DCM (20 mL) and DMF (20 mL) is stirred at room temperature overnight and the reaction progress is monitored by HPLC. The reaction mixture is filtered. The filtrate is washed twice with NaHCO₃ and the organic layer is dried over anhydrous MgSO₄. The solvent is removed in vacuo and the residue is purified by silica gel column chromatography to isolate the product.

Compound 4b: Compound 4b is prepared from compound 3b using the same conditions described for the preparation of compound 10a.

Compound 4c: Compound 4c is prepared from compound 3b using the same conditions described for the preparation of compound 10a.

### Example 7

### Preparation of Compound 5

Compound 5a: Compound 4a (0.744 mmol) is dissolved in 2 mL of anhydrous DCM, followed by addition of 1 mL of TFA dropwise at 0° C. The reaction mixture is stirred at 0° C. to room temperature for 30 minutes and concentrated in vacuo to give the product. The product is used as it is without further purification.

Compound 5b: Compound 5b is prepared from compound 4b using the same conditions described for the preparation of compound 10a.

Compound 5c: Compound 5c is prepared from compound 4b using the same conditions described for the preparation of compound 10a.

### Example 8

### Preparation of Compound 7

Compound 2 (135 mg, 0.405 mmol) was dissolved in 0.5 mL of DMF and 5 mL of DCM, followed by addition of compound 6 (306 mg, 1.62 mmol), DMAP (98 mg, 0.810 mmol) and pyridine (32 μL, 0.405 mmol). The reaction mixture was stirred overnight at room temperature and concentrated in vacuo. The residue was purified by column chromatography using ethylacetate-hexane (1:3, v/v) to give the product (90 mg). The structure of the product was confirmed by ¹H and ¹³C NMR.

### Example 9

### Preparation of Compound 8

Compound 7 (280 mg, 0.618 mmol) was dissolved in 4 mL of DCM and cooled in an ice bath, followed by addition of 1 mL of TFA dropwise at 0° C. The reaction mixture was stirred at 0° C. for 15 minutes and 1 hour at room temperature. The reaction mixture was concentrated in vacuo to give 210 mg (75% yield) of product. The product was used as it is without further purification. The structure of the product was confirmed by ¹H and ¹³C NMR.

### Example 10

### Preparation of Compound 10

Compound 10a: Compound 9 (4-arm PEG-tosylate, Mw. 40,000, 3.0 g, 0.075 mmol) is added to a solution of compound 5a (0.9 mmol) in a mixture of anhydrous DCM (30 mL) and anhydrous DMF (3 mL), followed by addition of DMAP (0.1 mmol) and DIEA (0.9 mmol) at 0° C. The reaction mixture is stirred at 0° C. to room temperature overnight and concentrated in vacuo. The resulting residue is recrystallized from IPA to give the product.

Compound 10b: Compound 10b is prepared from compound 5b using the same conditions described for the preparation of compound 10a.

Compound 10c: Compound 10c is prepared from compound 5b using the same conditions described for the preparation of compound 10a.

### Example 11

### Preparation of Compound 12

A mixture of compound 11 (614.5 mg, 3.26 mmol), compound 2 (500 mg, 1.55 mmol), and DIEA (388.6 mg) in anhydrous DCM (50 mL) and anhydrous DMF (10 mL) was stirred overnight at room temperature. The reaction mixture was filtered. The filtrate was washed twice with 0.2 N HCl. The organic layer was dried over anhydrous MgSO₄. The solvent was removed in vacuo. The resulting residue was purified by silica gel column chromatography to give 156 mg of product. The structure of the product was confirmed by ¹³C NMR.

### Example 12

### Preparation of Compound 14

Compound 12 and compound 13 (4-arm PEG-amine, Mw. 40,000, 1.0 g, 0.025 mmol) were dissolved in anhydrous DCM (10 mL) and anhydrous DMF (1 mL). EDC (48 mg, 0.25 mmol) and DMAP (48.8 mg, 0.4 mmol) were added to the mixture at 0 to 5° C. The reaction mixture was stirred at 0° C. to room temperature overnight. The solvent was removed in vacuo. The resulting residue was recrystallized from ether/DCM and IPA/acetonitrile to give the product (880 mg). The structure of the product was confirmed by ¹³C NMR.

### Example 13

### Preparation of Compound 15

Compound 14 (500 mg) was dissolved in anhydrous DCM (2 mL). A solution of 4N HCl in dioxane (2 mL) was added to the reaction mixture and the mixture was stirred at room temperature for 2 hours. The reaction was monitored by HPLC. Anhydrous ethyl ether was added to precipitate a crude product, which was recrustallized from ether-DCM to give the product (410 mg). The structure of the product was confirmed by ¹³C NMR.

### Example 14

### Preparation of Compound 17

Compound 17a: Compound 17a is prepared from compound 5a using the same conditions described for the preparation of compound 17d.

Compound 17b: Compound 17b is prepared from compound 5b using the same conditions described for the preparation of compound 17d.

Compound 17c: Compound 17c is prepared from compound 5c using the same conditions described for the preparation of compound 17d.

Compound 17d: Compound 16 (Mw. 40,000, 3.0 g, 0.075 mmol) was added to a solution of compound 8 (0.9 mmol) in a mixture of anhydrous DCM (30 mL) and anhydrous DMF (3 mL) at 0° C., followed by addition of DMAP (0.1 mmol) and DIEA (0.9 mmol). The reaction mixture was stirred at 0° C. to room temperature overnight and concentrated in vacuo. The resulting residue was recrystallized from IPA to give the product. The structure of the product was confirmed with ¹³C NMR.

### Example 15

### Preparation of Compound 19

Compound 19a: Compound 19a is prepared from compound 5a using the same conditions described for the preparation of compound 19d.

Compound 19b: Compound 19b is prepared from compound 5b using the same conditions described for the preparation of compound 19d.

Compound 19c: Compound 19c is prepared from compound 5c using the same conditions described for the preparation of compound 19d.

Compound 19d: Compound 18 (MW. 40,000, 3.0 g, 0.075 mmol) was added to a solution of compound 8 (0.9 mmol) in a mixture of anhydrous DCM (30 mL) and anhydrous DMF (3 mL) at 0° C., followed by addition of DMAP (0.1 mmol) and DIEA (0.9 mmol). The reaction mixture was stirred at 0° C. to room temperature overnight and concentrated in vacuo. The resulting residue was recrystallized from IPA to give the product. The structure of the product was confirmed with ¹³C NMR.

### Example 16

### Preparation of Compound 21

Compound 21 was prepared from compound 2 and compound 20 using the same conditions described for the preparation of compound 12. The structure of the product was confirmed with ¹³C NMR.

### Example 17

### Preparation of Compound 22

Compound 22 was prepared from compound 13 and compound 21 using the same conditions described for the preparation of compound 14. The structure of the product was confirmed with ¹³C NMR.

### Example 18

### Preparation of Compound 23

Compound 23 was prepared from compound 22 using the same conditions described for the preparation of compound 15. The structure of the product was confirmed with ¹³C NMR.

### Example 19

### Preparation of Compound 25

Compound 25a: Compound 25a is prepared from compound 5a using the same conditions described for the preparation of compound 25d.

Compound 25b: Compound 25b is prepared from compound 5b using the same conditions described for the preparation of compound 25d.

Compound 25c: Compound 25c is prepared from compound 5c using the same conditions described for the preparation of compound 25d.

Compound 25d: Compound 8 (0.2 mmol) and compound 24 (MW. 40,000, 1.0 g, 0.025 mmol) were dissolved in anhydrous DCM (10 mL) and anhydrous DMF (1 mL). EDC (48 mg, 0.25 mmol) and DMAP (48.8 mg, 0.4 mmol) were added at 0 to 5° C. The reaction mixture was stirred at 0° C. to room temperature overnight. The solvent was removed in vacuo. The resulting residue was recrystallized from ether/DCM and IPA/acetonitrile to give the product (880 mg). The structure of the product was confirmed by ¹³C NMR.

### Example 20

### Preparation of Compound 27

Compound 27a: Compound 27a is prepared from compound 5a using the same conditions described for the preparation of compound 25d.

Compound 27b: Compound 27b is prepared from compound 5b using the same conditions described for the preparation of compound 25d.

Compound 27c: Compound 27c is prepared from compound 5c using the same conditions described for the preparation of compound 25d.

Compound 27d: Compound 8 (0.2 mmol) and compound 26 (MW. 40,000, 0.025 mmol) were dissolved in anhydrous DCM (10 mL) and anhydrous DMF (1 mL). EDC (48 mg, 0.25 mmol) and DMAP (48.8 mg, 0.4 mmol) were added at 0 to 5° C. The reaction mixture was stirred at 0° C. to room temperature overnight. The solvent was removed in vacuo. The resulting residue was recrystallized from ether/DCM and IPA/acetonitrile to give the product. The structure was confirmed by ¹³C NMR.

### Example 21

### Preparation of Compounds 31a and 31b

Triphosgene (1.22 mmol) was added to a solution of compound 29a or 29b (3.05 mmol) in anhydrous DCM (4 mL), followed by addition of a solution of DMAP (6.12 mmol) in anhydrous DCM (4 mL) at 0° C. The mixture containing compound 30a or 30b was stirred for 2 hours and added to a mixture of SU5416 (compound 1, 243 mg, 1.02 mmol) and KOH powder (28.3 mg, 0.504 mmol) in DMF/THF (6 mL, 1:1, v/v) at 0° C. The reaction mixture was stirred in an ice-bath for 2 hours and concentrated in vacuo. The residue was purified by silica gel column chromatography using ethyl acetate-hexane (3:7, v/v) to provide compound 31a or 31b, respectively. The structure of the product was confirmed by ¹³C NMR.

### Example 22

### Preparation of Compounds 32a and 32b

Compound 31a or 31b (0.744 mmol) was dissolved in anhydrous DCM (6 mL), followed by addition of TFA (3 mL) dropwise at 0° C. The reaction mixture was stirred at 0° C. to room temperature for about 30 minutes. The reaction was monitored by HPLC. Upon completion of the reaction, anhydrous ethyl ether was added to precipitate the product. The product was collected by filtration, washed with anhydrous ethyl ether and dried in vacuo at room temperature to give a crude product. The crude product was used without further purification.

### Example 23

### Preparation of Compounds 34a and 34b

Compound 34a is prepared with compound 33 and compound 32a by the same conditions described for compound 34b.

Compound 34b: DMAP (0.05 mmol) and DIEA (0.2 mmol) were added to a solution of compound 33 (4-arm SC-PEG, Mw. 40,000, 1.0 g, 0.025 mmol) and compound 32b (0.245 mmol) in anhydrous DCM (20 mL) at 0° C. The reaction mixture was stirred 0° C. to room temperature overnight. Anhydrous ethyl ether was added to precipitate a crude product, which was collected by vacuum filtration and recrystallized twice with IPA/DMF to give compound 34b, respectively (yield, 0.95 g). The structure of the product was confirmed by ¹³C NMR.

### Example 24

### Preparation of Compound 37

To a solution of BOC-ext-OH (compound 35, 313.9 mg, 1.5 mmol), triphosgene (149.4 mg, 0.504 mmol) in 2 mL of anhydrous DCM, DMAP (184.7 mg, 1.5 mmol) was added at 0° C. and the mixture was stirred for two hours. The resulting solution was added into a solution of SU5416 potassium salt (compound 2, 100 mg, 0.420 mmol) in 6 mL of DMF/THF (1:1, v/v). Compound 28 was prepared by treating SU5416 with KOH powder (28.3 mg, 0.504 mmol) for one hour. The reaction mixture was stirred at 0° C. for about an hour and washed with 0.1N HCl twice. The organic layer was dried over anhydrous MgSO₄ and the solvent was removed in vacuo. The residue was purified by column chromatography using 50% EtOAc in hexane to give 200 mg of product. The structure of the product was confirmed with LC-MS and ¹³C, ¹H NMR.

### Example 25

### Preparation of Compound 38

Compound 37 (337 mg, 0.744 mmol) was dissolved in 2 mL of anhydrous DCM, followed by addition of 1 mL of TFA dropwise at 0° C. The reaction mixture was stirred at 0° C. to room temperature for 30 minutes and concentrated in vacuo to give the product. The product was used without further purification.

### Example 26

### Preparation of Compound 39

Compound 38 (0.412 mmol) was dissolved in 30 mL DCM, followed by addition of DIEA (0.061 mL, 0.35 mmol), DMAP (8.5 mg, 0.07 mmol), and compound 33 (1.3 g, 0.035 mmol) at 0° C. The reaction mixture was gradually warm to room temperature overnight. A solid product was obtained by adding ether. The solid was crystallized twice from IPA/DMF to give the product (1.2 g). The structure of the product was confirmed by ¹³C NMR.

### Example 27

### Preparation of Compounds 42a and 42b

Compound 42a is prepared from compound 1 and compound 41a under the same conditions described for compound 42b.

Compound 42b: To a solution of triphosgene (257.04 mg, 2.6 mmol) in 4.4 mL of anhydrous THF, TEA (595 μL, 4.3 mmol) was added at 0° C. and the mixture was stirred for 15 minutes at 0° C. Compound 1 (476 mg, 2.0 mmol) was added to the mixture at 0° C. and the resulting mixture was stirred at 0° C. to room temperature overnight. Compound 41b (8 mmol) and pyridine (8 mmol) were added to the mixture and the mixture was stirred for about 3 hours at room temperature. The reaction progress was monitored by HPLC. The reaction mixture was concentrated in vacuo and the resulting residue was purified by silica gel column chromatography using hexane-ethyl acetate (7:3 to 1:1, v/v) to give the product.

### Example 28

### Preparation of Compounds 43a and 43b

Compound 43a is prepared from compound 42a under the same conditions described for compound 43b.

Compound 43b: Compound 42b (160 mg, 0.311 mmol) was dissolved in 3.5 mL of anhydrous DCM, followed by addition of 1.75 mL of TFA dropwise at 0° C. The reaction mixture was stirred at 0° C. to room temperature for 30 minutes and concentrated in vacuo to give the product. The structure of the product was confirmed by ¹³C and ¹H NMR. The product was used as it is without further purification.

### Example 29

### Preparation of Compounds 44a and 44b

Compound 44a is prepared from compound 43a under the same conditions described for compound 44b.

Compound 44b: Compound 43b (0.315 mmol) was dissolved in a mixture of anhydrous DCM (15 mL) and anhydrous DMF (1.5 mL), followed by addition of DIEA (0.115 mL, 0.663 mmol), DMAP (0.6 mg, 0.006 mmol), and compound 33 (1.6 g, 0.040 mmol) at 0° C. The reaction mixture was stirred at 0° C. to room temperature overnight. Anhydrous ethyl ether was added to precipitate a crude product, which was collected by vacuum filtration and recrystallized twice with IPA/DMF to give the product (yield, 1.2 g). The structure of the product was confirmed by ¹³C NMR.

### Example 30

### Preparation of Compound 46a-d

Compound 46a: Compound 1 (200 mg, 0.84 mmol) and BocGly-OH (45a, 294 mg, 1.68 mmol) were dissolved in DCM (8 mL) and DMF (2 mL) and the mixture was cooled to 0-5° C. EDC (363 mg, 1.89 mmol) and DMAP (461 mg, 3.78 mmol) were added. The reaction mixture was stirred at 0° C. to room temperature and the reaction was monitored by HPLC. Upon completion of the reaction, the reaction mixture was washed with 1% NaHCO₃ twice and with 0.2 N HCl three times. The organic layer was dried over anhydrous Na₂SO₄ and the solvent was removed in vacuo completely to yield 0.326 g (98%) of product: ¹³C NMR δ 169.48, 167.34, 155.05, 138.15, 134.79, 134.42, 126.89, 126.39, 125.44, 124.10, 123.56, 115.91, 115.62, 113.30, 108.62, 79.66, 47.40, 28.80, 28.13, 14.57, 12.26.

Compound 46b is prepared from compound 1 and Boc-Ala-OH (compound 45b) by the same conditions for the preparation of compound 46a.

Compound 46c was prepared from compound 1 and Boc-Phe-OH (compound 45c) by the same conditions described for the preparation of compound 46a. The structure of the product was confirmed with ¹³C NMR in DMSO-d₆.

Compound 46d was prepared from compound 1 and Boc-Leu-OH (compound 45d) by the same conditions described for the preparation of compound 46a.

### Example 31

### Preparation of Compound 47a-d

Compound 47a: Compound 46a (0.300 g, 0.76 mmol) was suspended in DCM (3.5 mL) and the solution was cooled to 0° C., followed by addition of TFA (1.75 mL). The reaction mixture was stirred at 0° C. for about 30 minutes and the reaction was monitored by HPLC. Upon completion of the reaction, the reaction mixture was concentrated in vacuo in an ice water. Cold ether was added to precipitate solids. The solvent was decanted and solids were washed twice with cold ether. The product was dried in vacuo over P₂O₅ to yield 0.246 g (79%) of product: ¹³C NMR (DMSO-d₆) δ 165.94, 165.01, 137.83, 134.53, 132.64, 125.75, 125.38, 123.93, 123.29, 123.23, 115.31, 113.65, 112.43, 106.21, 43.26, 12.51, 10.26.

Compound 47b is prepared from compound 46b by the same conditions described for the preparation of compound 47a.

Compound 47c was prepared from compound 46c by the same conditions for the preparation of compound 47a. The structure of the product was confirmed with ¹³C NMR in DMSO-d₆.

Compound 47d was prepared from compound 46d by the same conditions described for the preparation of compound 47a.

### Example 32

### Preparation of Compound 48a-d

Compound 48a: Compound 33 (2.6 g, 0.064 mmol) and Compound 47a (0.209 g, 0.512 mmol) were dissolved in a mixture of anhydrous DCM (26 mL) and anhydrous DMF (2.6 mL). DIEA (0.188 mL, 0.139 g, 1.08 mmol) and DMAP (0.001 g, 0.010 mmol) were added at room temperature and the reaction mixture was stirred at room temperature overnight. The reaction mixture was concentrated in vacuo. Anhydrous ethyl ether was added to precipitate a crude product, which was collected by vacuum filtration and recrystallized from 5.2 mL acetonitrile/100 mL IPA and from 2 mL DMF/0.5 mL Acetonitrile/100 mL IPA. The product was collected by vacuum filtration and dried in vacuo at 35° C. to yield 2.37 g (90%) of product: ¹³C NMR δ 169.62, 167.96, 155.98, 138.56, 134.96, 134.82, 127.07, 126.56, 125.65, 124.35, 123.10, 116.08, 115.73, 113.50, 108.54, 78.01, 77.43, 76.08, 69.26-71.08 (PEG), 64.01, 47.20, 13.96, 11.59.

Compound 48b is prepared from compound 47b and compound 33 by the same conditions described for the preparation of compound 48a.

Compound 48c was prepared from compound 47c and compound 33 by the same conditions for the preparation of compound 48a in 87% yield: ¹³C NMR δ 11.71, 14.09, 30.82, 38.68, 56.04, 63.99, 69.27-70.32 (PEG), 71.13, 78.08, 108.74, 113.64, 115.89, 116.18, 124.22, 124.46, 125.69, 126.60, 127.20, 128.02, 129.26, 135.06, 135.24, 135.87, 138.68, 155.44, 167.65, 172.42.

Compound 48d was prepared from compound 47d and compound 33 by the same conditions described for the preparation of compound 48a.

### Example 33

### Preparation of Compound 50a-h

Compounds 50a and 50c, 50d, 50e, 50f, 50g, and 50h are prepared from 32a, 38, 43a, 43b, 47a, 47b, and 47c, respectively, by using the same conditions described for the preparation of compound 50b.

Compound 37b: Compound 49 (4-arm PEG acid, MW. 40,000, 1.0 g, 0.025 mmol) was azeotrophed for 1 hour in toluene and concentrated in vacuo. The resulting residue was dissolved in anhydrous DCM (20 mL) and cooled to 0° C. in an ice bath. EDC (38.4 mg, 0.2 mmol), DMAP (49 mg, 0.4 mmol), and compound 32b (0.25 mmol) were added to the solution at 0° C. and the mixture was stirred at 0° C. to room temperature overnight. Anhydrous ethyl ether was added to precipitate a crude product, which was collected by vacuum filtration and recrystallized twice with IPA/DMF to give the product (0.95 g). The structure of the product was confirmed with ¹³C NMR.

### Example 34

### Preparation of Compound 52

Compound 33 (3.0 g, 0.075 mmol) was added to a solution of 5-amino-2-pentanol (compound 51, 92.7 mg, 0.9 mmol) in a mixture of anhydrous DCM (30 mL) and anhydrous DMF (3 mL), followed by addition of DIEA (116 mg, 0.9 mmol). The reaction mixture was stirred at room temperature overnight and concentrated in vacuo. The resulting residue was recrystallized from IPA to give the product (2.75 g, 92% yield). The structure of the product was confirmed with ¹³C NMR.

### Example 35

### Preparation of Compound 53

Triphosgene (58.1 mg, 0.20 mmol) and pyridine (0.0475 mL, 0.59 mmol) were added to a solution of compound 52 (2.35 g, 0.059 mmol) in anhydrous chloroform (25 mL) at room temperature. The reaction mixture was stirred at 30° C. for about 4 hours, followed by addition of NHS (94.6 mg, 0.82 mmol) and pyridine (0.0665 mL, 0.82 mmol). The mixture was stirred at 30° C. for about 48 hours and concentrated in vacuo. The resulting residue was recrystallized from ether-DCM and IPA-acetonitrile to give the product (2.1 g, 89% yield). The structure of the product was confirmed with ¹³C NMR.

### Example 36

### Preparation of Compound 54

A mixture of SU5416 (compound 1, 71.4 mg, 0.3 mmol) and KOH powder (20.2 mg, 0.36 mmol) in DMF/THF (5 mL, 1:1, v/v) was stirred for 1 hour at 0° C. to form compound 28. The mixture was added to a solution of compound 53 (1.0 g, 0.025 mmol) in anhydrous DCM (10 mL) and the mixture was stirred overnight at room temperature. The solvent was removed in vacuo and the resulting residue was recrystallized from ethyl ether-DCM and IPA-acetonitrile to give the product (553 mg, 55% yield). The structure of the product was confirmed with ¹³C NMR.

### Example 37

### Preparation of Compound 55

A mixture of SU5416 (compound 1, 110 mg, 0.3 mmol), formaldehyde (˜37 wt % in water, 8 mL), and ammonium hydroxide (28-30 wt % ACS reagent grade, 2 mL) was stirred at 50° C. for about 5 hours and cooled to room temperature. A precipitate was formed, isolated by vacuum filtration, and washed with water several times. The solids were dissolved in chloroform, concentrated in vacuo, and dried in vacuo at 40° C. to give the product in 95% yield. The structure of the product was confirmed with ¹³C and ¹H NMR.

### Example 38

### Preparation of Compound 56

Compound 33 (1.0 g, 0.025 mmol) and compound 55 (80.1 mg, 0.3 mmol) were dissolved in a mixture of anhydrous DCM (9 mL) and anhydrous DMF (1 mL), followed by addition of DIEA (0.087 mL, 0.5 mmol). The reaction mixture was stirred at room temperature overnight and concentrated in vacuo. The resulting residue was recrystallized from DCM-ether and from IPA-acetonitrile to give the product (814 mg, 81% yield). The structure of the product was confirmed with ¹³C NMR.

### Example 39

### Preparation of Compound 57

A mixture of SU5416 (compound 1, 110 mg, 0.3 mmol) and formaldehyde (˜37 wt % in water, 10 mL) is stirred at 50° C. for about 5 hours and cooled to room temperature. A precipitate is formed, isolated by vacuum filtration, and washed with water several times. The solids are dissolved in chloroform, concentrated in vacuo, and dried in vacuo at 40° C. to give the product.

### Example 40

### Preparation of Compound 58

Compound 58 is prepared from compound 57 and Boc-Ala-OH (compound 45b) by the same conditions for the preparation of compound 46b.

### Example 41

### Preparation of Compound 59

Compound 59 is prepared from compound 58 by the same conditions for the preparation of compound 47b.

### Example 42

### Preparation of Compound 60

Compound 60 is prepared from compound 33 and compound 59 by the same conditions for the preparation of compound 56.

### Example 43

### Preparation of Compound 61

A mixture of SU5416 (compound 1, 71.4 mg, 0.3 mmol) and KOH powder (20.2 mg, 0.36 mmol) in DMF/THF (5 mL, 1:1, v/v) is stirred for 1 hour at 0° C. to form compound 28. The mixture is added to a solution of compound 16 (1.0 g, 0.025 mmol) in anhydrous DCM (10 mL) and the mixture is stirred overnight at room temperature. The solvent is removed in vacuo and the resulting residue is recrystallized from ethyl ether-DCM and IPA-acetonitrile to give the product.

### Example 44

### Preparation of Compound 62

Compound 62 was prepared from compound 33 and compound 28 by the same conditions for the preparation of compound 54.

### Example 45

### Regeneration of Parent Molecules From Compounds of Formula (I) or (I′)

The rate of hydrolysis was measured by monitoring disappearance of polymeric conjugates and appearance of the parent molecule by HPLC using the procedure, for example, as described in Example 4 in PBS and in rat plasma. In addition, using the procedure described in Example 3, the amounts of parent molecules (e.g., SU5416) in polymer conjugates was measured in % wt/wt and provided below.

The rate of hydrolysis was measured by monitoring disappearance of polymer conjugates and appearance of the parent molecule by HPLC using the procedure for example as described in Example 4 in PBS and in rat plasma.

TABLE 1

| Compound No | T_{1/2} in at plasma | | T_{1/2} in PBS | Loading (% w/w) |
|---|---|---|---|---|
| 15 | 56 | h | >72 h | ND |
| 17d | 7 | h | >72 h | ND |
| 23 | 57 | h | >72 h | ND |
| 25d | >72 | h | >72 h | ND |
| 27d | >72 | h | >72 h | ND |
| 34a | 55 | min | >72 h | 2.13 |
| 34b | 1.3 | h | >72 h | 2.30 |
| 39 | 2.3 | min | >72 h | 2.31 |
| 44b | >72 | h | >72 h | 1.95 |
| 48a | <1 | min | 28 h | 1.86 |
| 48c | 7.5 | min | >24 h | 1.91 |
| 48d | 2.7 | min | 2.7 h | 2.95 |
| 50b | 29 | min | >72 h | 2.22 |
| 62 | 1.15 | h | >72 h | 2.40 |

ND: Not determined

The hydrolysis result shows that the compounds of the invention are stable in PBS but released the parent molecule, SU5416, in various rate.

The loading efficiencies show that about four equivalents of SU5415 were conjugated to one equivalent of four-arm polymer.

We claim:

1. A compound of Formula (I), comprising: 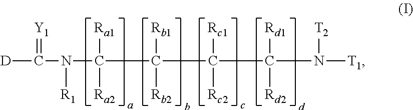
wherein
D is an amine-linked biologically active moiety or a hydroxyl-linked biologically active moiety;
Y₁ is O, S, or NR₅;
R₁ is hydrogen, C₁₋₆ alkyl, or aryl;
Rₐ₁, Rₐ₂, R_{b1}, R_{b2}, R_{c1}, R_{c2}, R_{d1}, and R_{d2}, in each occurrence, are independently selected from the group consisting of hydrogen, OH, C₁₋₆ alkyls, C₁₋₆ alkenyls, C₁₋₆ alkynyls, C₁₋₆ alkoxy, C₃₋₈ cycloalkyls, aryls, C(O)—R₆, targeting groups, substantially non-antigenic 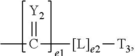
or two of Rₐ₁, R_{b1}, R_{c1}, and R_{d1} form a four to eight carbon-membered cyclic or heterocyclic ring, and optionally the two of Rₐ₁, R_{b1}, R_{c1}, and R_{d1} form a double bond;
T₁ is selected from the group consisting of hydrogen, C₁₋₆ alkyls, C₁₋₆ alkenyls, C₁₋₆ alkynyls, C₃₋₈ cycloalkyls, aryls, leaving groups, functional groups, targeting groups, and 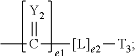
T₂ is selected from the group consisting of hydrogen, C₁₋₆ alkyls, C₁₋₆ alkenyls, C₁₋₆ alkynyls, C₃₋₈ cycloalkyls, aryls, functional groups and targeting groups;
Y₂ is O, S, or NR_{S};
L, in each occurrence, is the same or different bifunctional linking moiety;
T₃ is selected from the group consisting of hydrogen, OH, amine, halogen, C₁₋₆ alkyls, C₁₋₆ alkenyls, C₁₋₆ alkynyls, C₁₋₆ alkoxy, C₃₋₈ cycloalkyls, aryls, leaving groups, functional groups, targeting groups, and substantially non-antigenic polymers;
R₅ and R₇ are independently hydrogen, C₁₋₆ alkyl, or aryl;
R₆ is OH, C₁₋₆ alkyl, aryl, C₁₋₆ alkoxy, or aryloxy;
(a), (b), (c), and (d) are independently zero or one, and the sum of (a), (b), (c) and (d) is one, two, three or four; and
(e1) is zero or one;
(e2) is zero or a positive integer of from about 1 to about 6; and
provided that T₁ is 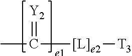
or a leaving group, wherein L contains a releasable linker and (e2) is a positive integer of from about 1 to about 6, when T₂ is not hydrogen; and provided that Rₐ₁, Rₐ₂, R_{b1}, R_{b2}, R_{c1}, R_{c2}, R_{d1}, and R_{d2}, in each occurrence, are not all hydrogen, when T₁ and T₂ are both hydrogen.

2. The compound of claim 1 having the formula:

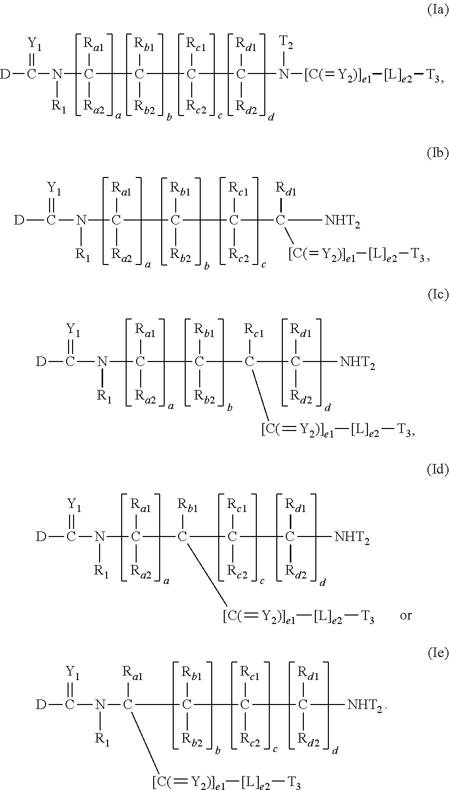

3. The compound of claim 1 having the formula: 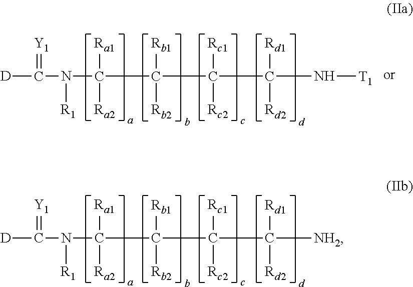
wherein in formula (IIa) T₁ is selected from the group consisting of hydrogen, C₁₋₆ alkyls, C₁₋₆ alkenyls, C₁₋₆ alkynyls, C₃₋₈ cycloalkyls, aryls, leaving groups, functional groups, targeting groups, and [L]ₑ₂-T₃ and 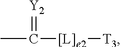
and
in formula (IIb) one of Rₐ₁, Rₐ₂, R_{b1}, R_{b2}, R_{c1}, R_{c2}, R_{d1} and R_{d2} is selected from the group consisting of targeting groups, substantially non-antigenic polymers, and 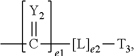
wherein T₃ is not hydrogen when (e1) and (e2) are each zero; and
T₃ is selected from the group consisting of hydrogen, OH, amine, halogen, C₁₋₆ alkyls, C₁₋₆ alkenyls, C₁₋₆ alkynyls, C₁₋₆ alkoxy, C₃₋₈ cycloalkyls, aryls, leaving groups, functional groups, targeting groups and substantially non-antigenic polymers.

4.-8. (canceled)

9. The compound of claim 1 having Formula (I′), comprising: 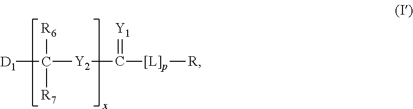
wherein
D₁ is an indolinone-containing kinase inhibitor, wherein D₁ is linked via the indolinone amine;
R is a substantially non-antigenic polymer;
L, in each occurrence, is the same or different bifunctional linker;
R₆ and R₇ are independently hydrogen or C₁₋₄ alkyls;
Y₁ is O, S or NH;
Y₂ is O, S or NH;
(x) is zero or 1; and
(p) is zero or a positive integer of from about 1 to about 6.

10. The compound of claim 9, wherein the compound has the formula: 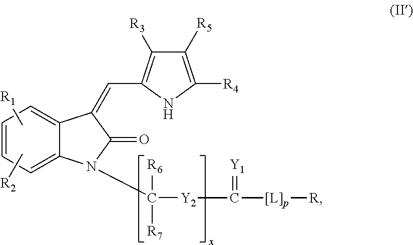
wherein
R is a substantially non-antigenic polymer;
L, in each occurrence, is the same or different bifunctional linker;
R₁ and R₂ are independently selected from the group consisting of hydrogen, halogen, alkyls, alkylthio, nitro, trihalomethyl, hydroxy, hydroxyalkyls, alkoxys, cyano, aryl, —C(O)R₁₁, NR₁₂R₁₃, —NR₁₂C(O)R₁₃, —SO₂R₁₂, and —S(O)₂NR₁₂R₁₃,
wherein R₁₁ is selected from the group consisting of alkyls, amino, hydroxy, alkoxys, aryl, aryloxy, and aminoalkylamino; and R₁₂ and R₁₃ are independently selected from the group consisting of hydrogen, alkyls, and aryl;
R₃ is selected from the group consisting of hydrogen, alkyls, hydroxyalkyls, aminoalkyls, —C(O)R₁₁, and aryl;
R₄ is selected from the group consisting of hydrogen, alkyls, —C(O)R₁₁, and aryl;
R₅ is selected from the group consisting of hydrogen, —CH₂CH₂COOH, —COR₁₄, and —CH₂CH₂C(O)NR₁₅R₁₆, wherein
(a) when R₅ is —COR₁₄, R₁₄ is selected from the group consisting of alkyls, alkoxys, hydroxy, aryl, aryloxy, alkylamino, dialkylamino, and —NR₃₁R₃₂,
wherein R₃₁ is hydrogen or an alkyl; and R₃₂ is selected from the group consisting of aminoalkyls, hydroxyalkyls, acetylalkyls, cyanoalkyls, carboxyalkyls, and alkoxycarbonylalkyls; and wherein the alkyl in the aminoalkyls is optionally substituted with one or two hydroxyl group(s); and
(b) when R₅ is —CH₂CH₂C(O)NR₁₅R₁₆,
(i) R₁₅ is hydrogen or a C₁₋₄ alkyl; and R₁₆ is -A₁-NR₃₃R₃₄,
wherein R₃₃ and R₃₄ are independently hydrogen or C₁₋₄ alkyls; and A₁ is (CH₂)ₐ₁, (CH₂)ₐ₂-A₂-(CH₂)ₐ₃ or (CH₂CH₂O)ₐ₄—CH₂CH₂, wherein (a1) is an integer of from about 2 to about 10; (a2) and (a3) are independently selected integers of from about 1 to about 6; A₂ is CH═CH, phenylene, biphenylene, cyclohexylene or piperazinylene; and (a4) is 1, 2 or 3; or
(ii) R₁₅ and R₁₆ together form -A₃-NR₃₅-A₄-,
wherein R₃₅ is hydrogen or a C₁₋₄ alkyl; and A₃ and A₄ are independently (CH₂)ₐ₅ or (CH₂CH₂O)ₐ₆CH₂CH₂, wherein (a5) is an integer of from about 2 to about 6; and (a6) is 1, 2 or 3; or
(iii) R₁₅ and R₁₆ together with the nitrogen atom to which they are attached form a piperidinyl, wherein the piperidinyl group bears a substituent of formula -A₅-R₃₆ at the 4 position, wherein A₅ is C₁₋₄ alkylene; and R₃₆ is piperidin-4-yl; or
(iv) R₁₅ and R₁₆ together with the nitrogen atom to which they are attached form pyrrolidinyl, piperidinyl or morpholino; or
R₄ and R₅ together form —(CH₂)₄— or —(CH₂)ₐ₇CO(CH₂)ₐ₈—, wherein (a7) is 0, 1, 2, or 3; (a8) is 0, 1, 2, or 3, provided that the sum of (a7) and (a8) is 3;
R₆ and R₇ are independently hydrogen or C₁₋₄ alkyls;
Y₁ is O, S or NH;
Y₂ is O, S or NH;
(x) is zero or 1; and
(p) is zero or a positive integer of from about 1 to about 6.

11. The compound of claim 1 wherein the substantially non-antigenic polymer is a polyalkylene oxide.

12. The compound of claim 11, wherein the polyalkylene oxide is selected from the group consisting of polyethylene glycol, polypropylene glycol, and combinations thereof.

13. The compound of claim 11, wherein the polyalkylene oxide comprises a polyethylene glycol of the formula:
—(CH₂CH₂O)ₙ— or —[C(═O)]_{f2}—(CH₂)_{f1}-M₁-CH₂CH₂(OCH₂CH₂)ₙ—
wherein
M₁ is O, S, or NH;
(f1) is zero or a positive integer of from about 1 to about 10;
(f2) is zero or one; and
(n) is an integer from about 10 to about 2,300.

14. (canceled)

15. The compound of claim 11, selected from the group consisting of:
(IIIa) 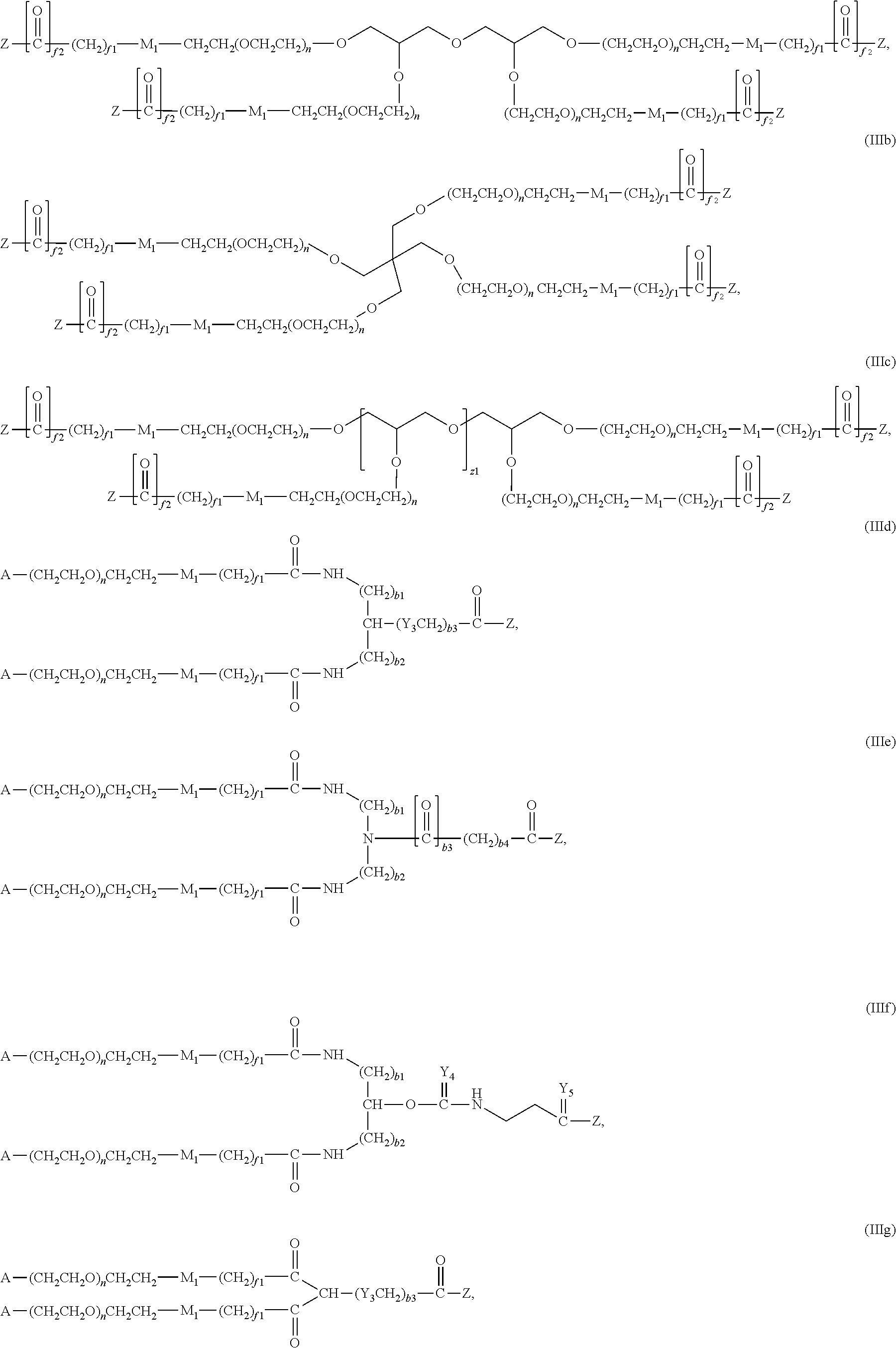
Z—[C(═O)]_{f2}—(CH₂)_{f1}-M₁-CH₂CH₂—O—(CH₂CH₂O)ₙ—CH₂CH₂-M₁-(CH₂)_{f1}-[C(═O)]_{f2}—Z, (IIIh)
and
A-(CH₂CH₂O)ₙ—CH₂CH₂-M₁-(CH₂)_{f1}-[C(═O)]_{f2}—Z, (IIIi)
wherein
A is hydroxyl, NH₂, CO₂H, or a C₁₋₆ alkoxy;
M₁ is O, S, or NH;
Y₃ is O, NR₅₁, S, SO or SO₂;
Y₄ and Y₅ are independently O, S or NR₅₁;
R₅₁, in each occurrence, is independently hydrogen, C₁₋₈ alkyl, C₁₋₈ branched alkyl, C₁₋₈ substituted alkyl, aryl, or aralkyl;
Z, in each occurrence, is independently selected from the group consisting of OH, a leaving group, a targeting group, C₁₋₈ alkyl, C₁₋₈ alkoxy, an aryl, 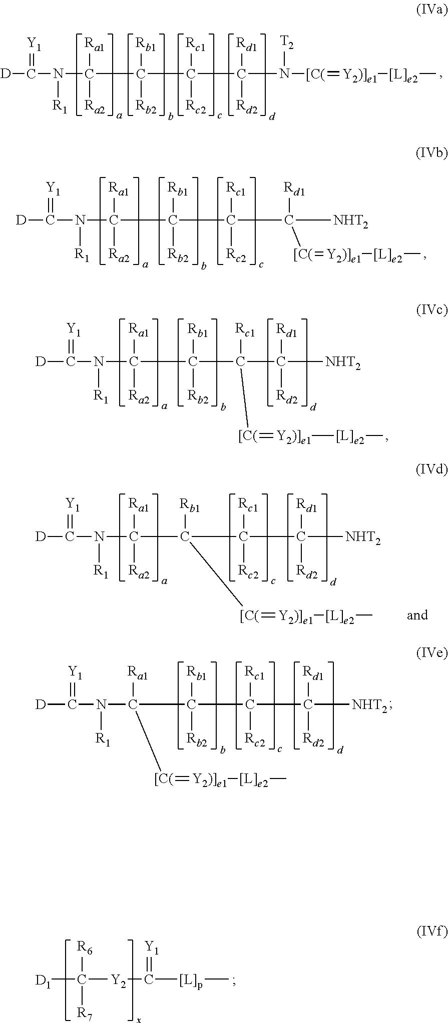
wherein
T₂ is selected from the group consisting of hydrogen, C₁₋₆ alkyls, C₁₋₆ alkenyls, C₁₋₆ alkynyls, C₃₋₈ cycloalkyls, and aryls;
(b1) and (b2) are independently zero or a positive integer;
(b3) is zero or 1;
(b4) is a positive integer;
(f1) is zero or a positive integer of from about 1 to about 10;
(f2) is zero or one;
(z1) is zero or a positive integer of from 1 to about 27;
(n) is a positive integer of from about 10 to about 2,300 so that the polymeric portion of the compound has the total number average molecular weight of from about 2,000 to about 100,000 daltons;
(x) is zero or 1; and
(p) is zero or a positive integer of from about 1 to about 6, preferably 1, 2, 3;
provided that one or more Z are (IVa), (IVb), (IVc), (IVd), (IVe) or (IVf).

16. (canceled)

17. The compound of claim 1, wherein L is L₁ selected from the group consisting of
—[C(═O)]ᵥ(Y₂₁)ᵤ₁(CR₂₁R₂₂)ₜ₁(Y₂₂)ᵤ₂—,
—[C(═O)]ᵥ(Y₂₁)ᵤ₁(CR₂₃R₂₄O)ₜ₃—,
—[C(═O)]ᵥ(Y₂₁)ᵤ₁(CR₂₃R₂₄O)ₜ₃(CR₂₁R₂₂)ₜ₁(Y₂₂)ᵤ₂—,
—[C(═O)]ᵥ(Y₂₁)ᵤ₁(CR₂₁R₂₂)ₜ₁(CR₂₃R₂₄O)ₜ₃—,
—[C(═O)]ᵥ(Y₂₁)ᵤ₁(CR₂₁R₂₂)ₜ₁Y₂₃(CR₂₁R₂₂)ₜ₂—,
—[C(═O)]ᵥ(Y₂₁)ᵤ₁(CR₂₅R₂₆CR₂₇R₂₈O)ₜ₄—,
—[C(═O)]ᵥ(Y₂₁)ᵤ₁(CR₂₅R₂₆CR₂₇R₂₈O)ₜ₄(CR₂₁R₂₂)ₜ₁(Y₂₂)ᵤ₂—,
—[C(═O)]ᵥ(Y₂₁)ᵤ₁(CR₂₁R₂₂)ₜ₁(CR₂₅R₂₆CR₂₇R₂₈O)ₜ₄—,
—[C(═O)]ᵥ(Y₂₁)ᵤ₁(CR₂₁R₂₂)ₜ₁Y₂₃(CR₂₅R₂₆CR₂₇R₂₈O)ₜ₄—, 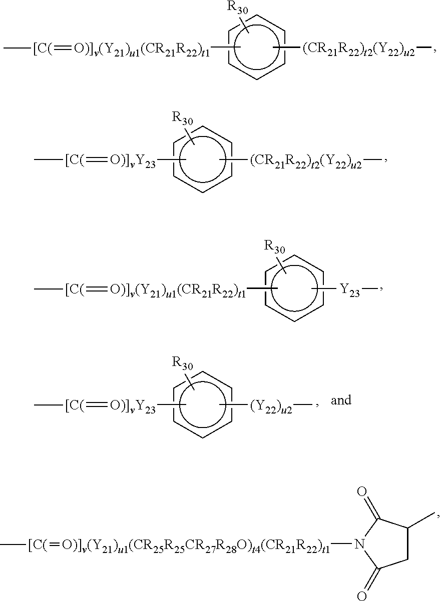
wherein
R₂₁-R₃₀ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy;
Y₂₁ is O, S or NR₂₉;
Y₂₂ and Y₂₃ are independently O, S or NR₂₉;
(t1) and (t2) are independently positive integers;
(t3) is a positive integer;
((t4) is a positive integer;
(u1) and (u2) are independently zero or 1; and
(v) is zero or 1,
provided that (v) is zero in the first L₁ adjacent to C(═Y₂), when (e1) is a positive integer.

18. The compound of claim 1 wherein L is L₂ which is a residue of an amino acid or amino acid derivative, or a peptide, and C(═Y₂) together with L₂ is selected from the group consisting of 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine, sarcosine, N-methyl-isoleucine, 6-N-methyl-lysine, N-methylvaline, norvaline, norleucine, and ornithine.

19. The compound of claim 1 wherein L is L₃ which has the formula: 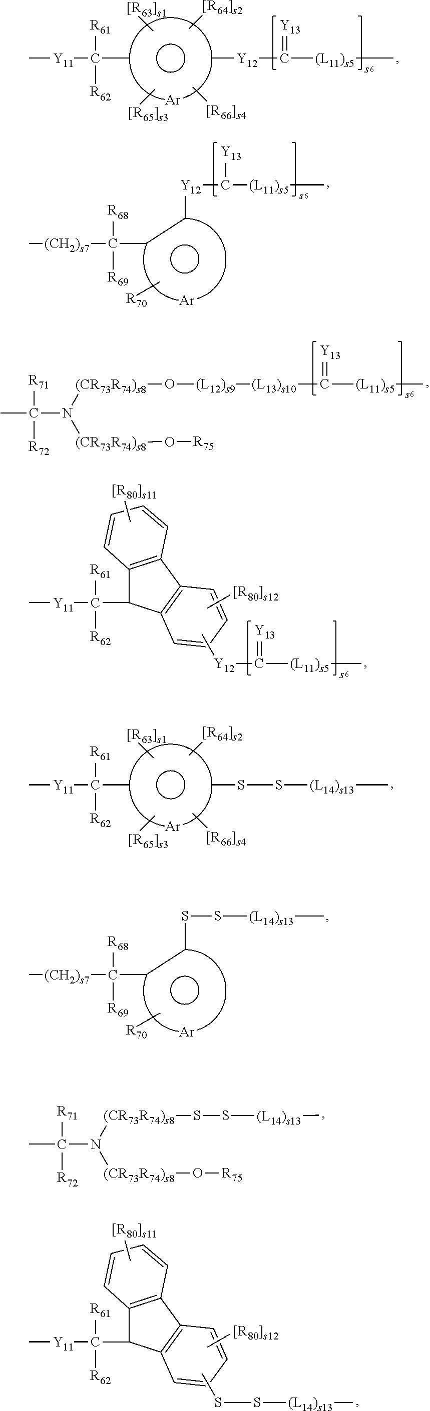
wherein
Y₁₁ is O, or S;
Y₁₂ is O, S, or NH, provided that L₁₁ is Gly-Phe-Leu-Gly, Ala-Leu-Ala-Leu, Phe-Lys, or Val-Cit, when Y₁₂ is NH and (s6) is a positive integer;
Y₁₃ is O, S, or NR₆₇;
L₁₁ and L₁₃ are independently bifunctional linking moiety, and the same as defined as L₁ and L₂;
L₁₂ is
—C(O)CR₇₆R₇₇OCR₇₆R₇₇C(O)—;
—C(O)CR₇₆R₇₇NR₇₈CR₇₆R₇₇C(O)—;
—C(O)CR₇₆R₇₇SCR₇₆R₇₇C(O)—, or
—C(O)(CR₇₆R₇₇)ₛ₁₁C(O)—;
L₁₄ is a bifunctional linking moiety, and the same as defined as L₁ and L₂;
R₆₁, R₆₂, R₆₇, R₇₁, R₇₂, R₇₃ and R₇₄ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls;
R₆₃, R₆₄, R₆₅ and R₆₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;
R₆₈, R₆₉ and R₇₀ are independently selected from the group consisting of C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
R₇₅ is H, —C(═O)—R₇₉, wherein R₇₉, in each occurrence, is the same or different alkyl, 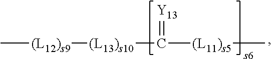
or a targeting group;
R₇₆, R₇₇ and R₇₈ are independently selected from the group consisting of from H, C₁₋₆ alkyl, C₂₋₆ alkenyl, C₂₋₆ alkynyl, C₁₋₆ heteroalkyl and aryl;
R₈₀, in each occurrence, is independently selected from the group consisting of SO₃H, NO₂, F, Cl, Br, I, CN, C(O)—R₇₉, COOH, COOR₇₉, CHO, COR₇₉, N(R₇₉)₃⁺, CF₃, and CCl₃;
Ar is a moiety which when included in Formula (I) forms an aromatic or heteroaromatic hydrocarbon;
(s1), (s2), (s3), and (s4) are independently zero or one;
(s5) is a positive integer of from about 1 to about 6;
(s6) is zero or one;
(s7) is zero, one or two;
(s8) is 1, 2 or 3;
(s9) is zero or one;
(s10) is zero or a positive integer of from about 1 to about 6;
(s11) and (s12) are independent zero, 1 or 2; and
(s13) is a positive integer.

20.-21. (canceled)

22. The compound of claim 1 wherein the polymer has the total number average molecular weight from about 2,000 to about 100,000 daltons.

23. The compound of claim 1 wherein the polymer has the total number average molecular weight of from about 5,000 to about 60,000 daltons.

24. The compound of claim 1 wherein the polymer has the total number average molecular weight from about 5,000 to about 25,000 daltons or from about 20,000 to about 45,000 daltons.

25. A compound as in claim 1 selected from the group consisting of: 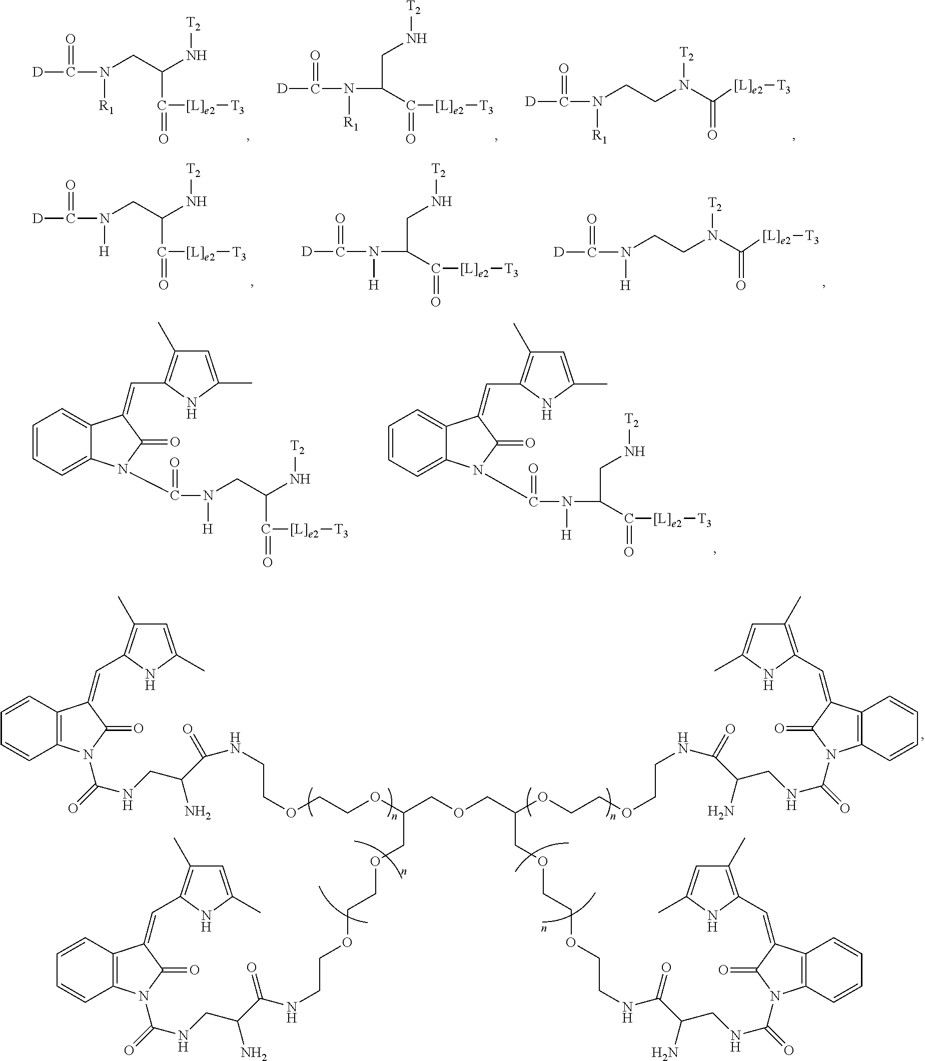 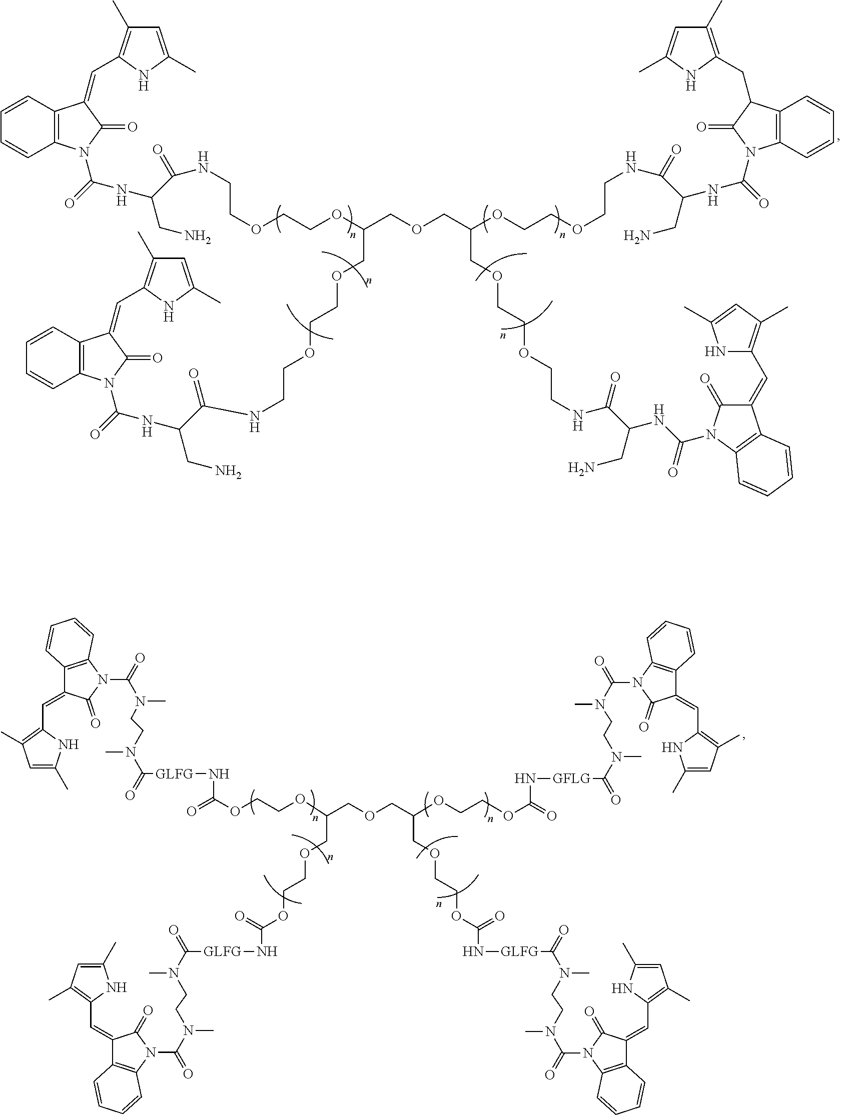 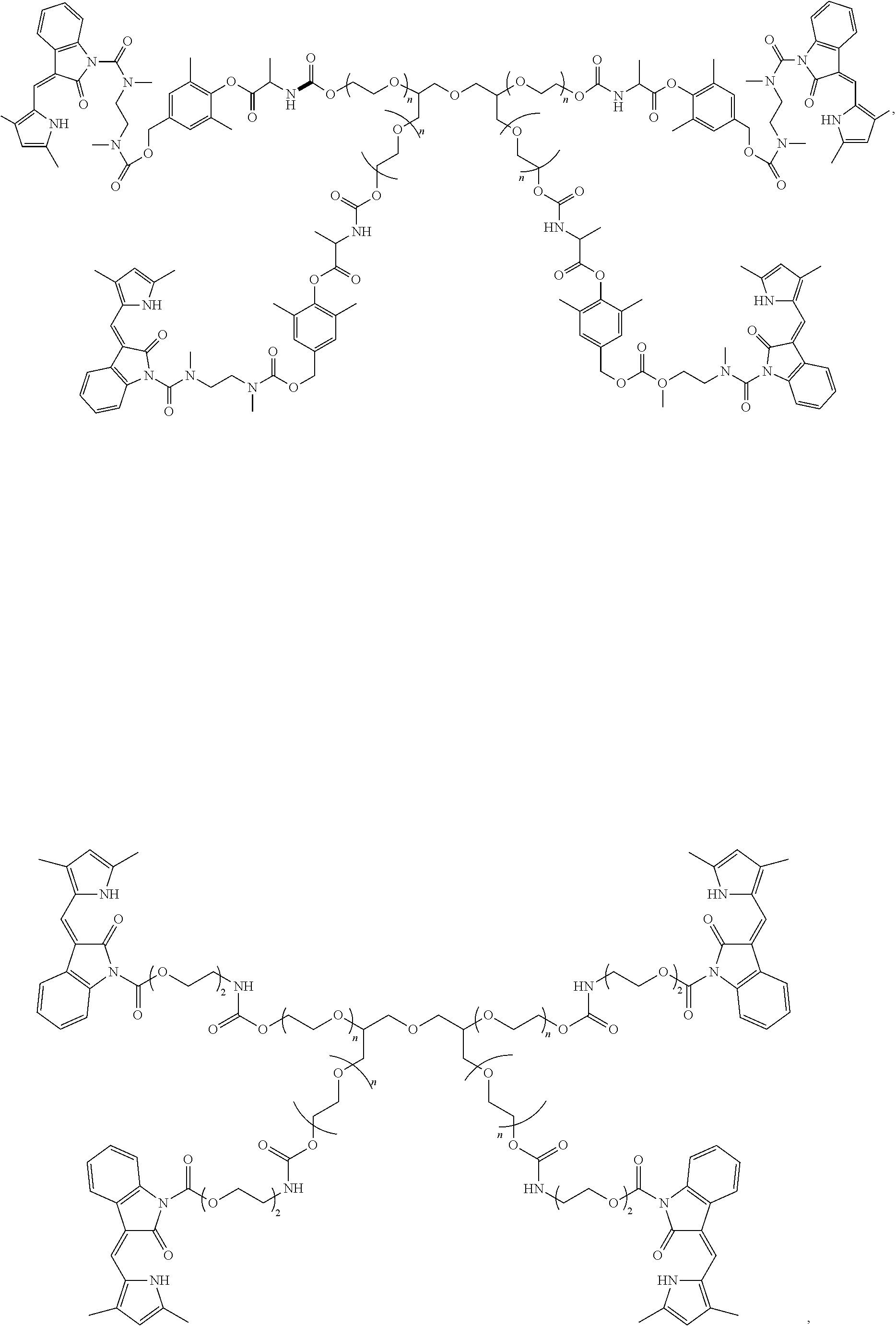 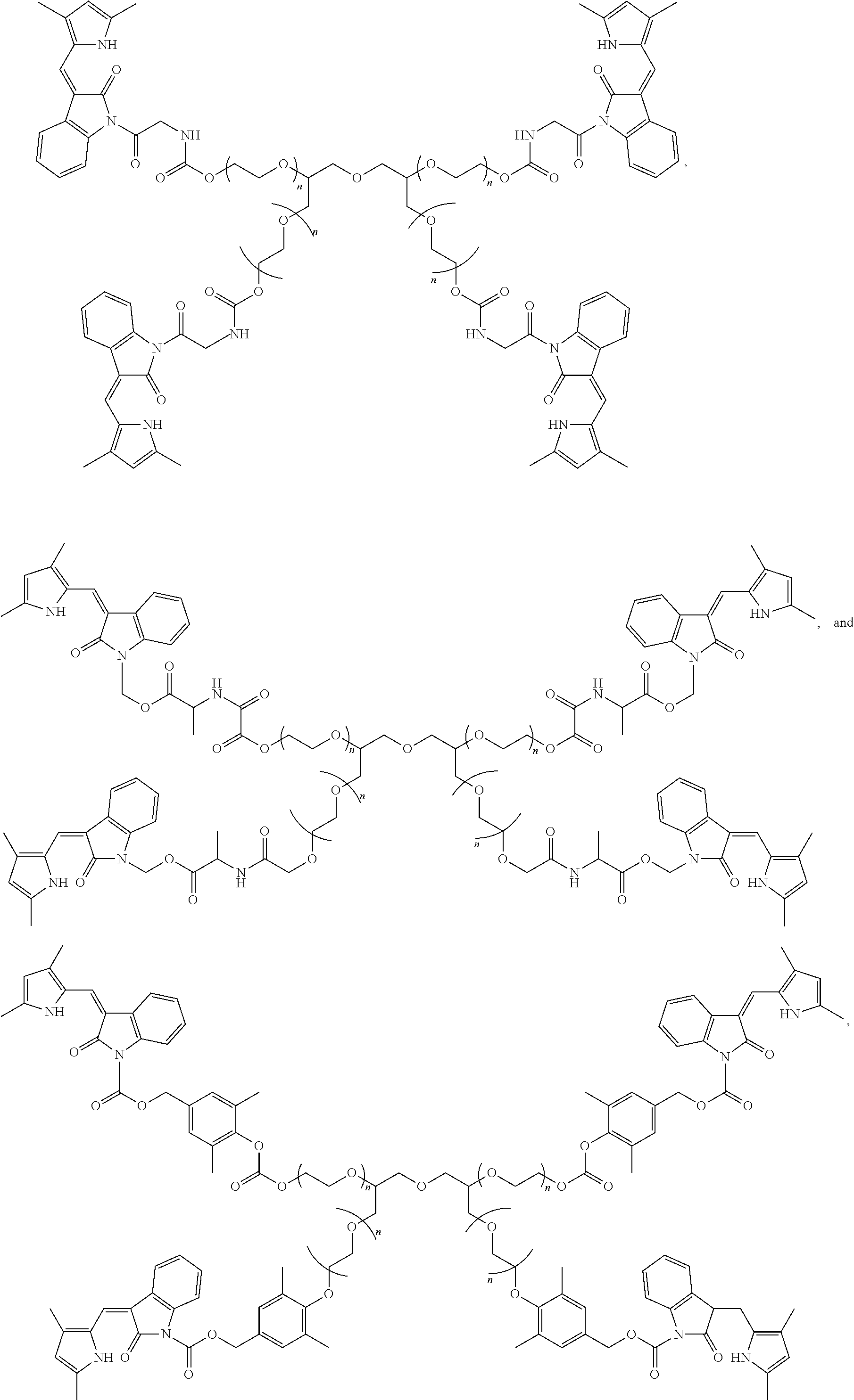
wherein (n) is an integer from about 10 to about 2,300.

26.-29. (canceled)

30. A method of delivering an aromatic amine-containing biologically active agent to a mammal, comprising
(a) forming a polymeric conjugate of an aromatic amine-containing biologically active agent or a polymeric conjugate of an indolinone-based tyrosine kinase inhibitor; and
(b) administering the conjugate to a mammal in need thereof, wherein the conjugate is represented by Formula (I) of claim 1.

31. (canceled)

32. A method of inhibiting angiogenesis or angiogenic activity in a mammal, comprising:
administering a compound of claim 1 or pharmaceutical salt thereof to a mammal in need thereof, wherein D is an indolinone-based tyrosine kinase inhibitor.

33. (canceled)

34. The method of claim 32, wherein the compound of Formula (I) of claim 1 is administered in an amount of from about 70 to about 150 mg/m³/dose and the amount is based on the indolinone-based tyrosine kinase inhibitor.

35. (canceled)

36. A method of treating a cancer in a mammal or inhibiting growth or proliferation of cancer cells, comprising administering a compound of claim 1 to a mammal in need thereof, wherein D is an indolinone-based tyrosine kinase inhibitor.

37.-39. (canceled)
